(19) United States
(12) Patent Application Publication
(10) Pub. No.: US 2006/0194321 A1
(43) Pub. Date: Aug. 31, 2006

(54) Directed differentiation of embryonic stem cells and uses thereof

(76) Inventors: Alan Colman, Helios (SG); William Sun, Helios (SG); Norris Ray Dunn, Helios (SG); Blaine Phillips, Helios (SG); Hentze Hannes Martin, Helios (SG); William Lathrop Rust, Helios (SG)

Correspondence Address: FISH & NEAVE IP GROUP;ROPES & GRAY LLP, ONE INTERNATIONAL PLACE, BOSTON, MA, 02110-2624, US

(21) Appl. No.: 11/342,957

(22) Filed: Jan. 30, 2006

Related U.S. Application Data
(60) Provisional application No. 60/648,640, filed on Jan. 31, 2005.
(60) Provisional application No. 60/691,954, filed on Jun. 17, 2005.
(60) Provisional application No. 60/753,431, filed on Dec. 22, 2005.

(51) Int. Cl. C12N 5/00; C12N 5/02

(52) U.S. Cl. 435/377; 435/395; 435/325

(57) ABSTRACT

The present invention provides methods for the directed differentiation of embryonic stem cells along the endodermal lineage, especially the pancreatic lineage.

### REFERENCE TO RELATED APPLICATIONS

This application claims the benefit of the filing dates of U.S. Provisional Application Ser. Nos. 60/648,640, filed on Jan. 31, 2005; 60/691,954, filed on Jun. 17, 2005; and 60/753,431 filed on Dec. 22, 2005. The teachings of the referenced applications are incorporated herein by reference.

### BACKGROUND OF THE INVENTION

Over the last decade, tremendous excitement in the stem cell field has fueled the hope that various stem cell populations will form the basis of treatments for a diverse array of degenerative diseases and disorders. Embryonic stem cells have attracted particular excitement for their seemingly unprecedented ability to differentiate to tissues derived from all three germ layers. Accordingly, embryonic stem cells may form the basis of a wider range of therapeutics than adult stem cells derived from any particular tissue.

However, despite the excitement generated by the limitless potential of embryonic stem cells to differentiate along ectodermal, mesodermal, and endodermal lineages, effective therapeutics require the ability to control and direct the differentiation of embryonic stem cells to a particular cell type. Furthermore, effective therapeutics require that this directed differentiation efficiently yields a particular differentiated cell type. In other words, it is advantageous for methods of directed differentiation to yield a high percentage of a particular differentiated cell type or to yield a high percentage of cell types that comprise a particular tissue or organ. Such efficient methods of differentiation represent a substantial leap from prior art methods which either fail to consistently yield particular cell types or yield an exceedingly low percentage of a particular differentiated cell type.

There exists a tremendous need to supply realistic therapeutic alternatives to the wide range of degenerative diseases and injuries affecting tissues derived from the ectoderm, mesoderm, or endoderm. Embryonic stem cells are a particularly attractive resource for developing such diverse therapies. Accordingly, the present invention provides methods of promoting the directed differentiation of embryonic attractive resource for developing such diverse therapies. Accordingly, the present invention provides methods of promoting the directed differentiation of embryonic stem cells to particular endodermally derived cell types. Such methods can be used to generate cultures of partially and/or terminally differentiated cells that can be used therapeutically to treat or prophylactically treat injuries and diseases of endodermally derived tissues and organs.

### SUMMARY OF THE INVENTION

The invention provides methods for the directed differentiation of cells to endodermal cell types. Endodermal cell types differentiated according to the methods of the present invention can be used to treat or prophylactically treat injuries and diseases of endodermally derived tissues and organs.

The present invention provides methods for directing the differentiation of embryonic stem cells to various endodermal cell types. Specifically, the present invention provides methods for directing the differentiation of embryonic stem cells along a pancreatic lineage. In certain embodiments, the methods of the invention lead to the production, from embryonic stem cells, of pdx-1⁺ cells indicative of cells that have begun differentiation to a pancreatic cell fate. In certain other embodiments, the methods of the invention lead to the production, from embryonic stem cells, of insulin producing cells. In still other embodiments, the methods of the invention lead to the production, from embryonic stem cells, of cells that express insulin and C-peptide, and/or are glucose-responsive.

Pdx-1⁺ cells and/or insulin-producing cells produced using the methods of the present invention can be delivered to human or animal patients and used for the treatment or prophylaxis of conditions of the pancreas.

Thus in one respect, the invention provides a method for directed differentiation of embryonic stem (ES) cells into pancreatic lineage, comprising: contacting the ES cells for a sufficient period of time with a sufficient amount of one or more early factors (EFs) selected from activin A, BMP2, BMP4, or nodal, wherein the pancreatic lineage cells express pancreatic lineage marker(s), and/or exhibit a pancreatic lineage function.

In certain embodiments, the pancreatic lineage cells express Pdx-1 and/or insulin, and/or are responsive to glucose, and/or secret C-peptide. Such pancreatic lineage cells may be Insulin-producing cells, such as pancreatic β-cells.

In certain embodiments, the ES cells are cultured as embryoid bodies (EBs), plated directly onto a support matrix (such as MATRIGEL™), and/or plated directly onto tissue culture plates. For example, the EBs may be cultured in a floating suspension culture, in a support matrix (such as MATRIGEL™ or other matrix), and/or on a filter.

Supporting matrices other than MATRIGEL™ are known in the art, including basement membrane extractable from placenta as described in Kawaguchi et al., Proc. Natl. Acad. Sci. 95(3): 1062-66, 1998; BD Bioscience's PuraMatrix synthetic peptide scaffold; or fibronectin matrix, etc.

In certain embodiments, the EBs are cultured in a support matrix (such as MATRIGEL™), only during the period when the EBs are in contact with the EFs.

In certain embodiments, the EBs are generated from ES cells grown on MEF (mouse embryonic feeder) or other feeder layers, or from ES cells grown under feeder-free conditions.

In a preferred embodiment, the ES cells are xeno-free, preferably also CGMP- and GTCP-compliant (CGMP: Current Good Manufacturing Practice; GTCP: Good Tissue Culture Practice).

ES cells from many different species of animals may be used in the methods of the invention. In certain embodiments, the ES cells are human ES cells. In other embodiments, the ES cells are from non-human mammals, such as ES cells from rodents (rats, mice, rabbits, hamsters, etc.); primates (e.g., monkey, apes, etc.), pets (cats, dogs, etc.); livestock animals (cattle, pigs, horses, sheep, goats, etc.).

In certain embodiments, the human ES cells are from the hES1, hES2, hES3, hES4, hES5, hES6 or DM ES cell lines.

In certain embodiments, the ES cells are partially or terminally differentiated into the pancreatic lineage.

In other embodiments, the ES cells are contacted with the EFs for about 15 days, preferably about 10 days, or about 1, 2, 3, 4, 5, 6, 7, 8, 9, 10, 11, 12, 13, or about 14 days.

In certain embodiments, the EFs comprise activin A and BMP4. In certain embodiments, the EFs comprise about 50 ng/mL (e.g., about 10-200 ng/mL, or about 20-100 ng/mL, or about 30-70 ng/mL, or about 40-60 ng/mL) of activin A and about 50 ng/mL (e.g., about 10-200 ng/mL, or about 20-100 ng/mL, or about 30-70 ng/mL, or about 40-60 ng/mL) of BMP4.

In certain embodiments, the method further comprises contacting the ES cells, subsequent to contacting the ES cells with the EFs, with a sufficient amount of one or more late factors (LFs) for a second sufficient period of time. For example, the LFs may be HGF, exendin4, betacellulin, and nicotinamide. In certain embodiments, the one or more LFs include about 50 ng/mL (e.g., about 10-200 ng/mL, or about 20-100 ng/mL, or about 30-70 ng/mL, or about 40-60 ng/mL) of HGF, about 10 ng/mL (e.g., about 2-50 ng/mL, or about 5-20 ng/mL) of exendin4, and about 50 ng/mL (e.g., about 10-200 ng/mL, or about 20-100 ng/mL, or about 30-70 ng/mL, or about 40-60 ng/mL) of β-cellulin.

In certain embodiments, the ES cells are contacted with the EFs for about 10 days, and are subsequently contacted with the LFs for about 10 days.

In certain embodiments, the EFs comprise about 50 ng/mL of activin A and about 50 ng/mL of BMP4, and the LFs include about 50 ng/mL of HGF, about 10 ng/mL of exendin4, and about 50 ng/mL of β-cellulin.

In certain embodiments, the method further comprises contacting the ES cells, subsequent to the initiation protocol and during a maturation protocol, consecutively with: (1) a basal medium for about 6 days; (2) about 20 ng/ml (e.g., about 5-100 ng/mL, or about 1040 ng/mL) FGF-18, and about 2 μg/ml (e.g., about 0.5-10 μg/ml, or about 1-5 μg/ml) Heparin in the basal medium for about 5-6 days; (3) about 20 ng/ml (e.g., about 5-100 ng/mL, or about 1040 ng/mL) FGF-18, about 2 μg/ml (e.g., about 0.5-10 μg/ml, or about 1-5 μg/ml) Heparin, about 10 ng/ml EGF (e.g., about 2-50 ng/mL, or about 5-20 ng/mL), about 4 ng/ml TGF-α (e.g., about 1-20 ng/mL, or about 2-10 ng/mL), about 30 ng/ml (e.g., about 5-150 ng/mL, or about 15-60 ng/mL) IGF1, about 30 ng/ml (e.g., about 5-150 ng/mL, or about 15-60 ng/mL) IGF2, and about 10 ng/ml (e.g., about 2-50 ng/mL, or about 5-20 ng/mL) VEGF in the basal medium for about 4-5 days; (4) about 10 μM (e.g., about 2-50 μM, or 5-20 μM) Forskolin, about 40 ng/ml (e.g., about 10-150 ng/mL, or about 20-80 ng/mL) HGF, and about 200 ng/ml (e.g., about 50-800 ng/mL, or about 100-400 ng/mL) PYY for about 3-4 days; and, (5) about 100 ng/ml (e.g., about 25-400 ng/mL, or about 50-200 ng/mL) Exendin-4, and about 5 mM (e.g., about 1-20 mM, or 2-10 mM) Nicotinamide for about 3-4 days.

In certain embodiments, steps (1)-(3) use DMEM/F12 medium or equivalents. In certain embodiments, step (4) uses RPMI 1640 or equivalent medium. In certain embodiments, step (5) uses CMRL medium. In certain embodiments, the ES cells are not dissociated by dispase between step (1) and (2). In certain embodiments, FBS (if any is used) in the medium is replaced with a chemically defined serum replacer (SR).

In certain embodiments, the method further comprises contacting the ES cells, subsequent to the EF and LF treatment, and during a maturation protocol, with about 10 μM (e.g., about 2-50 μM, or 5-20 μM) Forskolin, about 40 ng/ml (e.g., about 10-150 ng/mL, or about 20-80 ng/mL) HGF, and about 200 ng/ml (e.g., about 50-800 ng/mL, or about 100-400 ng/mL) PYY for about 3-4 days.

In certain embodiments, the ES cells are grown on fibronectin-coated tissue culture surfaces during the maturation protocol.

In certain embodiments, the differentiated cells release C-peptide and/or are responsive to glucose stimulation.

In certain embodiments, the method further comprises contacting the ES cells, subsequent to contacting the ES cells with the EFs and during a maturation protocol, consecutively with: (1) about 20 ng/ml FGF-18, and about 2 μg/ml Heparin in a basal medium for about 8 days; (2) about 20 ng/ml FGF-18, about 2 μg/ml Heparin, about 10 ng/ml EGF, about 4 ng/ml TGFα, about 30 ng/ml IGF1, about 30 ng/ml IGF2, and about 10 ng/ml VEGF in the basal medium for about 6 days; and (3) about 10 μM Forskolin, about 40 ng/ml HGF, and about 200 ng/ml PYY for about 5 days. It is contemplated that the range of concentrations of the factors are as those described above.

In certain embodiments, the differentiated cells release C-peptide.

In certain embodiments, step (1) above lasts 6 days, steps (2) and (3) last 4 days each.

In a second aspect, the invention provides cells and cell clusters differentiated by the methods of the present invention from embryonic stem cells. In one embodiment, the cells or cell clusters express pdx-1. In another embodiment, the cells or cell clusters express insulin. In still another embodiment, the cells or cell clusters express and secrete C-peptide. In yet another embodiment, the cells or cell clusters express both insulin and C-peptide. In any of the foregoing, exemplary cells or cell clusters are glucose-responsive.

Thus this aspect of the invention provides differentiated pancreatic lineage cells or cell cultures obtained through the various subject methods.

In certain embodiments, the differentiated pancreatic lineage cells or cell cultures are partially differentiated.

In other embodiments, the differentiated pancreatic lineage cells or cell cultures are terminally differentiated.

In certain embodiments, the differentiated pancreatic lineage cells or cell cultures mimic the function, in whole or in part, of Insulin-producing cells, such as pancreatic beta islet cells.

In a third aspect, the invention provides methods for the treatment or prophylaxis of diseases, injuries, or conditions of the pancreas. Such diseases, injuries, or conditions of the pancreas are characterized by impaired pancreatic function, for example, impaired ability to properly regulate glucose metabolism in an affected individual. In one embodiment, the disease, injury, or condition of the pancreas is diabetes (e.g., type I or type II diabetes), and the invention provides methods for the treatment or prophylaxis of diabetes. In one embodiment, the method of treatment comprises administering a composition of partially differentiated cells or cell clusters (e.g., pdx-1⁺). In another embodiment, the method of treatment comprises administering a composition of terminally differentiated cells or cells clusters. Such terminally differentiated cells or cell clusters comprises (in whole or in part) glucose responsive cells. In any of the foregoing, the invention contemplates methods of treatment comprising administration of cells or cell clusters differentiated by the methods of the invention along with one or more additional therapies.

Thus this aspect of the invention provides a method for the treatment or prophylaxis, in an individual, of diseases, injuries, or conditions of the pancreas characterized by impaired pancreatic function, comprising administering to the individual the subject differentiated pancreatic lineage cells.

In certain embodiments, the impaired pancreatic function includes impaired ability to properly regulate glucose metabolism in an affected individual.

In certain embodiments, the condition is type I or type II diabetes.

In certain embodiments, the method is in conjunction with one or more additional therapies effective for the treatment or prophylaxis of the diseases, injuries, or conditions.

In a fourth aspect the invention provides initiation protocols, maturation protocols, and combinations of initiation and maturation protocols for the directed differentiation of embryonic stem cells along a pancreatic lineage. In one embodiment, the initiation protocol, maturation protocol, or combination thereof promotes expression of pdx-1, insulin, and/or C-peptide. In another embodiment, the initiation protocol, maturation protocol, or combination thereof promotes induction of glucose responsive cells or cell clusters that mimic the function, in whole or in part, of beta islet cells.

In any of the foregoing, the invention contemplates that these methods can be used to direct the differentiation of other adult and fetal stem cell populations to endodermal cell types.

The embodiments of the invention, even when described for different aspects of the invention, are contemplated to be applicable for all aspects of the invention where appropriate.

Other features and advantages of the invention will be apparent from the following detailed description, and from the claims.

### DETAILED DESCRIPTION OF THE DRAWINGS

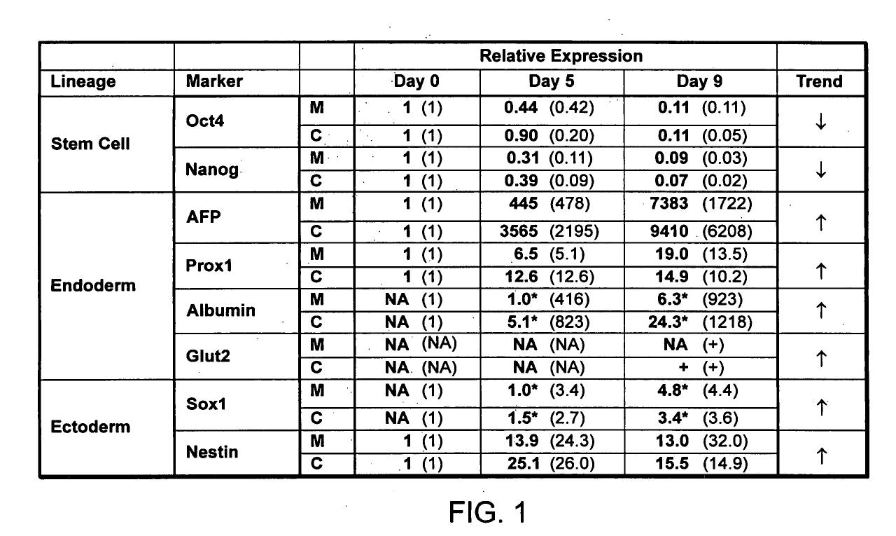

FIG. 1 shows the results of RT-PCR analysis of human embryonic stem cells allowed to spontaneously differentiate via embryoid body formation. Expression analysis confirmed that human embryonic stem cells can spontaneously differentiate along ectodermal, mesodermal, and endodermal lineages.

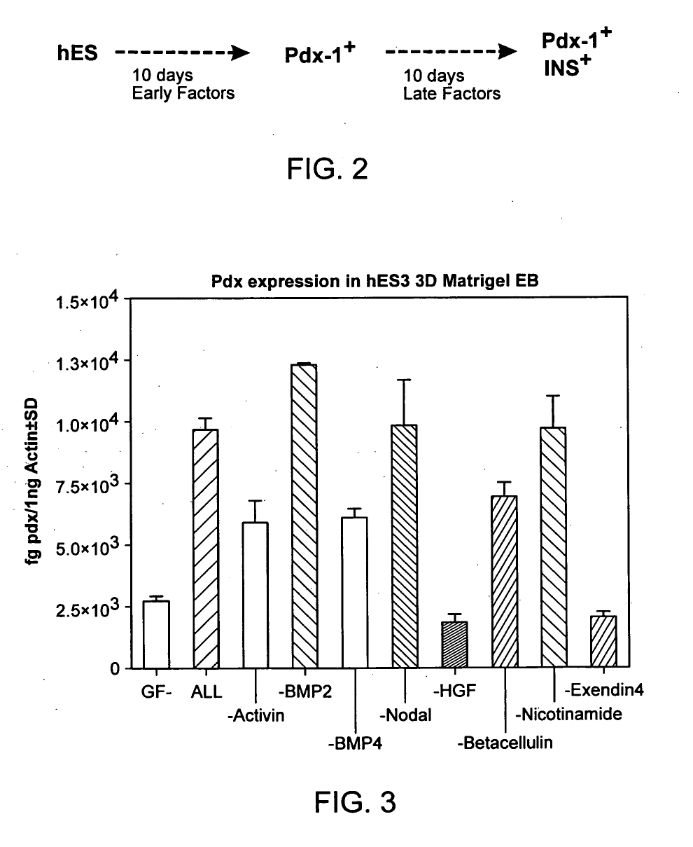

FIG. 2 shows a schematic representation of methods of directing differentiation of a stem cell to a differentiated pancreatic cell. The method proceeds in two stages. In the first stage, the stem cells are directed to differentiate along a particular lineage, for example the pancreatic lineage, by promoting expression of a marker indicative of partial differentiation down a particular lineage. In the second stage, the partially differentiated cells are terminally differentiated to express one or more markers of a particular differentiated cell type. Note that either partially or terminally differentiated cells may be therapeutically useful, and thus the method contemplates variations in which the end point (e.g., the goal of a particular differentiation protocol) is either generation of partially differentiated cells or the generation of terminally differentiated cells.

FIG. 3 summarizes the results of experiments in which hES3 were cultured as embryoid bodies suspended in 3D in MATRIGEL™. The cells were cultured for 10 days in medium containing the early factors and then for 10 days in medium containing the late factors. Following culture, cells were assayed for expression of pdx-1. For each bar depicted in FIG. 3, the embryoid bodies were cultured, except as indicated, with the following early and late factors: early factors were activin A, BMP2, BMP4, and nodal; late factors were HGF, exendin4, betacellulin, and nicotinamide. The particular factor omitted is indicated under each bar.

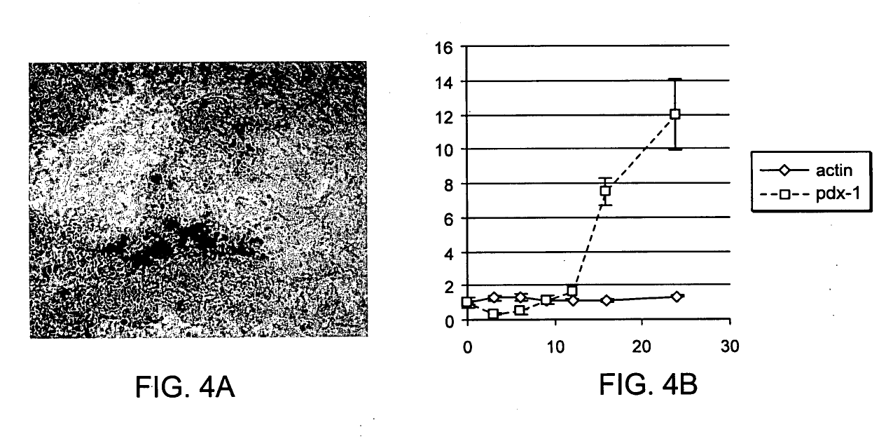

FIGS. 4A and 4B show the directed differentiation of a mouse embryonic stem cell along a particular endodermal lineage. A mouse embryonic stem cell line with lacZ reporter knocked into the pdx-1 locus was used to differentiate into pancreatic cells. FIG. 4A shows a cluster of cells expressing β-galactosidase (indicating pdx-1 expression) after EB formation and subsequent plating. FIG. 4B shows quantitative RT-PCR data for pdx-1 for mouse embryoid bodies at various stages of culture. Pdx-1 expression increased over time up to 24 days of EB formation.

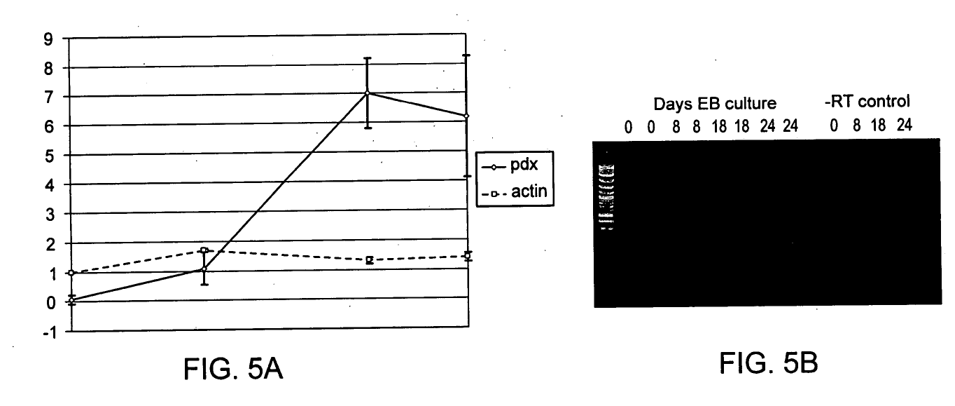

FIGS. 5A and 5B show that expression of the early pancreatic marker, pdx-1, increased over time in embryoid bodies formed from human embryonic stem cell line hES2. FIG. 5A shows that pdx-1 expression increased between 0-24 days of embryoid body formation, as measured by RT-PCR. As a control, actin expression was measured and this expression did not change significantly over time. FIG. 5B shows an ethidium bromide stained gel of the pdx-1 RT-PCR product, indicating that a single band of the predicted size was detected.

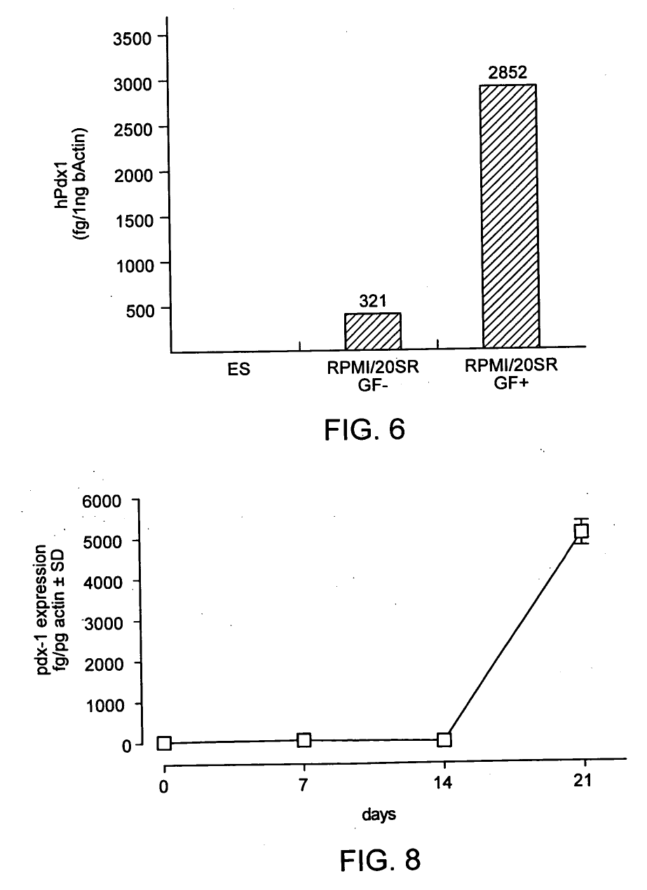

FIG. 6 shows that addition of TGFβ family growth factors to embryoid bodies, in culture, increased expression of pdx-1. Human ES cell line 3 (hES3) derived embryoid bodies were cultured in MATRIGEL™ in RPMI media supplemented with serum replacement. Expression of pdx-1 by RT-PCR was measured after 20 days in culture. Expression is expressed as fg per ng actin. Addition of TGFβ family growth factors resulted in a 9-fold increase in pdx-1 expression.

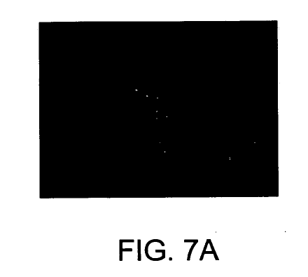

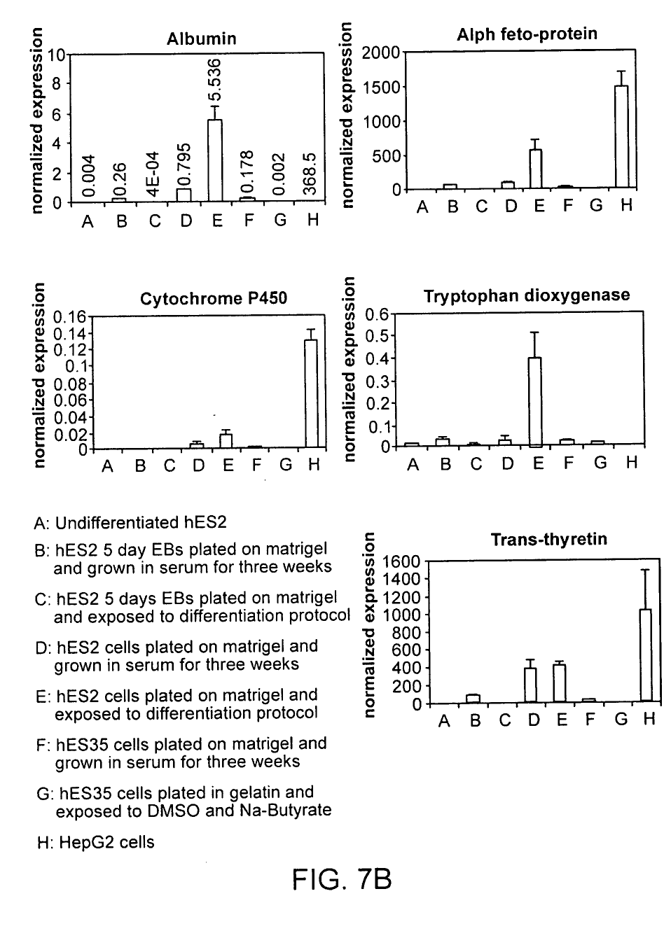

FIGS. 7A and 7B show the directed differentiation along a particular endodermal lineage. FIG. 7A shows a cluster of embryonic stem cells expressing the hepatocyte marker albumin. FIG. 7B shows quantitative RT-PCR data examining markers of endodermal differentiation in two different human embryonic stem cell lines undergoing any of several differentiation protocols.

FIG. 8 shows pdx-1 expression in embryonic stem cells at various time points during culture as embryoid bodies suspended in 3D culture in MATRIGEL™. The cells were cultured for 10 days in medium containing the early factors and then for 10 days in medium containing the late factors.

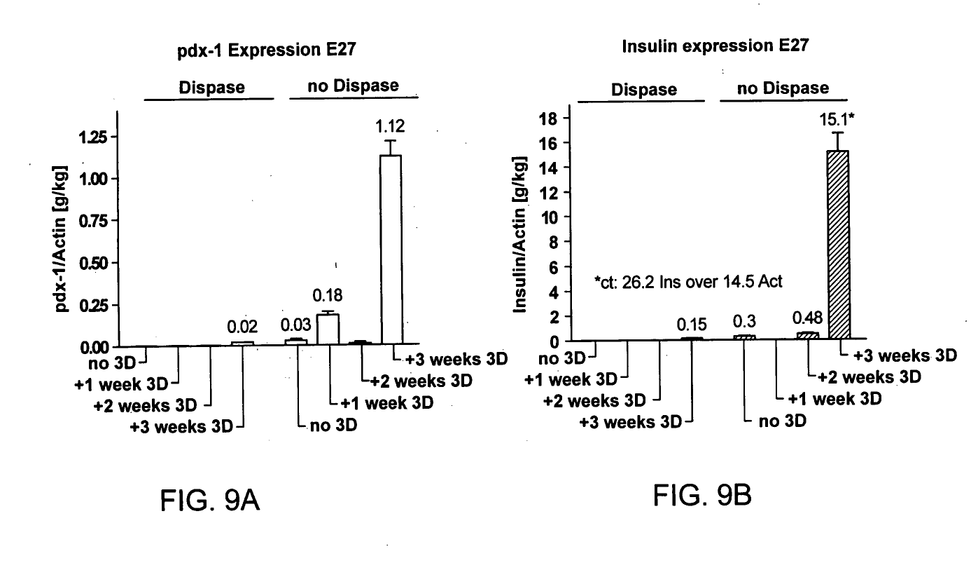

FIGS. 9A and 9B show expression of pdx-1 and insulin in embryonic stem cells differentiated under a combination of conditions. Cells were cultured as embryoid bodies in 3D cultures for three weeks in the presence of early and late factors, and were then subjected to a 27 day, multi-step differentiation protocol. FIG. 9A shows expression of pdx-1 and FIG. 9B shows expression of insulin.

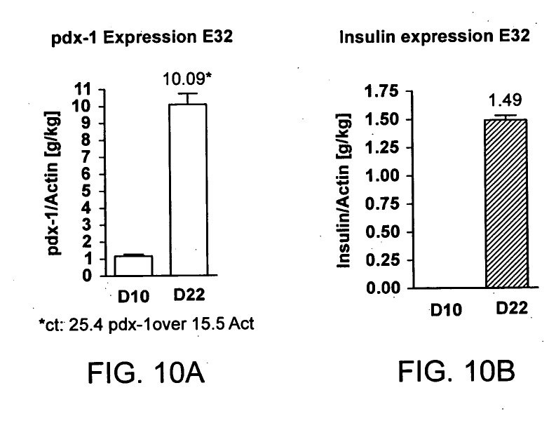

FIGS. 10A and 10B show expression of pdx-1 and insulin in embryonic stem cells differentiated under a combination of conditions. Cells were cultured as embryoid bodies in 3D cultures for one week, and were then subjected to a 32 day, multi-step differentiation protocol. FIG. 10A shows expression of pdx-1 and FIG. 10B shows expression of insulin.

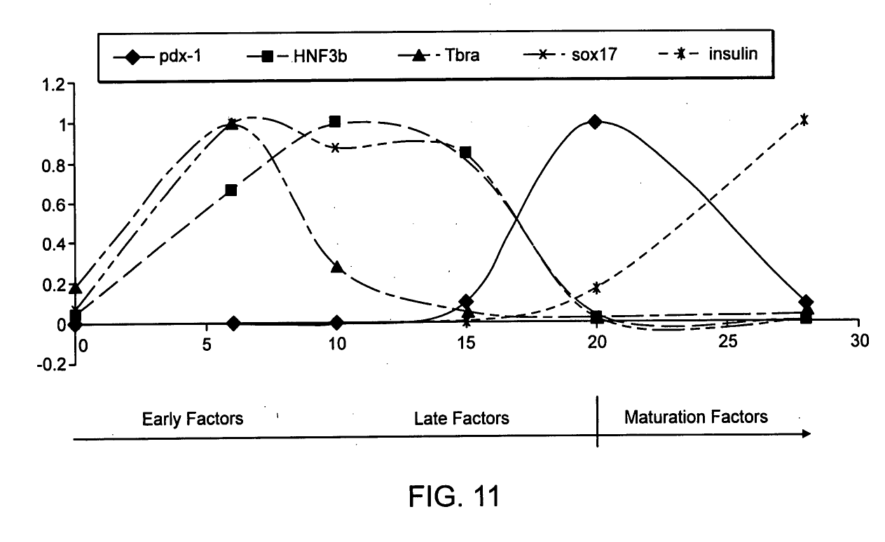

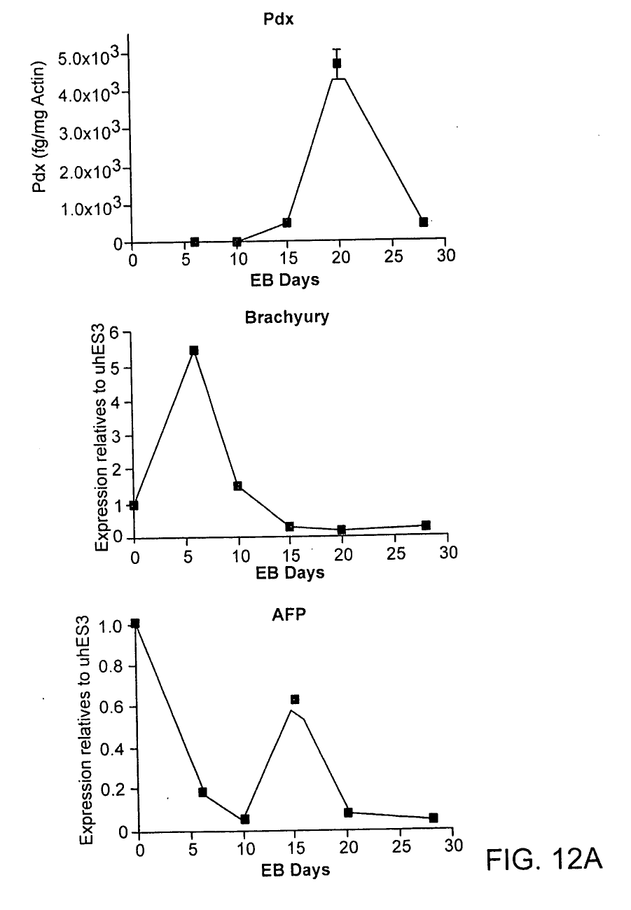

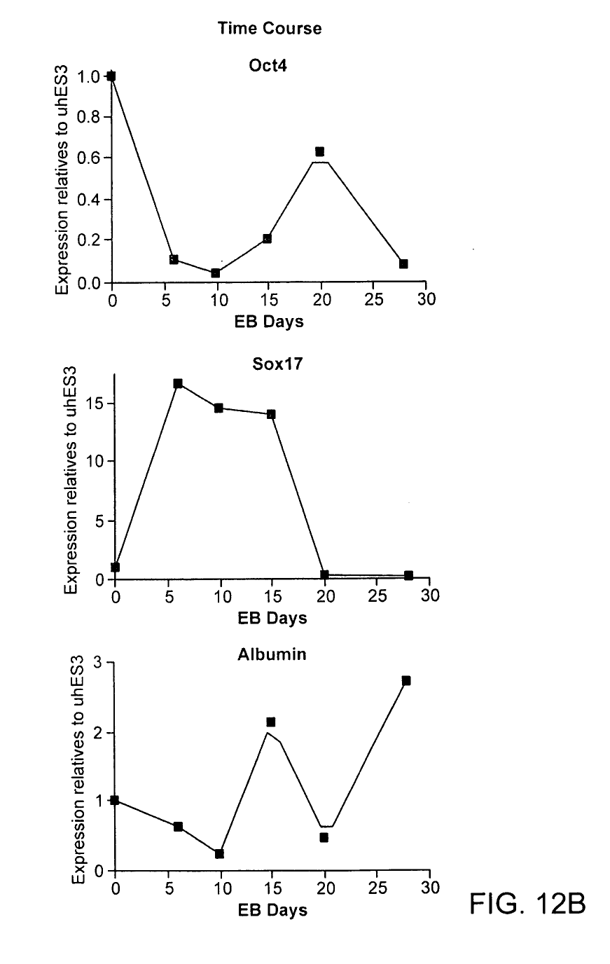

FIG. 11 shows the kinetics of endodermal and pancreatic gene expression during in vitro, directed differentiation of embryonic stem cells.

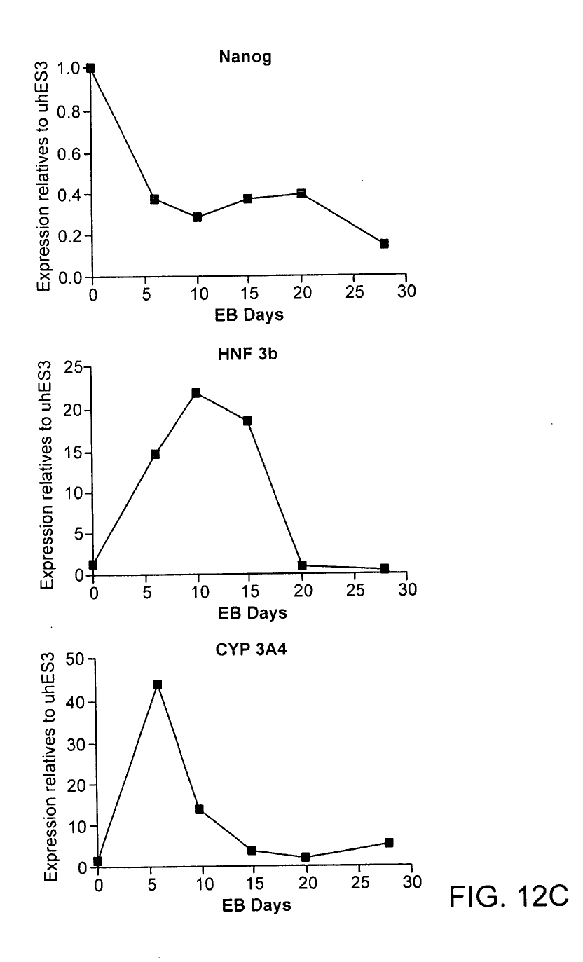

FIG. 12 shows a detailed analysis of the temporal pattern of gene expression during in vitro, directed differentiation of embryonic stem cells. The data summarized in FIGS. 11 and 12 demonstrate that gene expression during the directed differentiation of embryonic stem cells along a pancreatic lineage mimics that which occurs during normal pancreatic development.

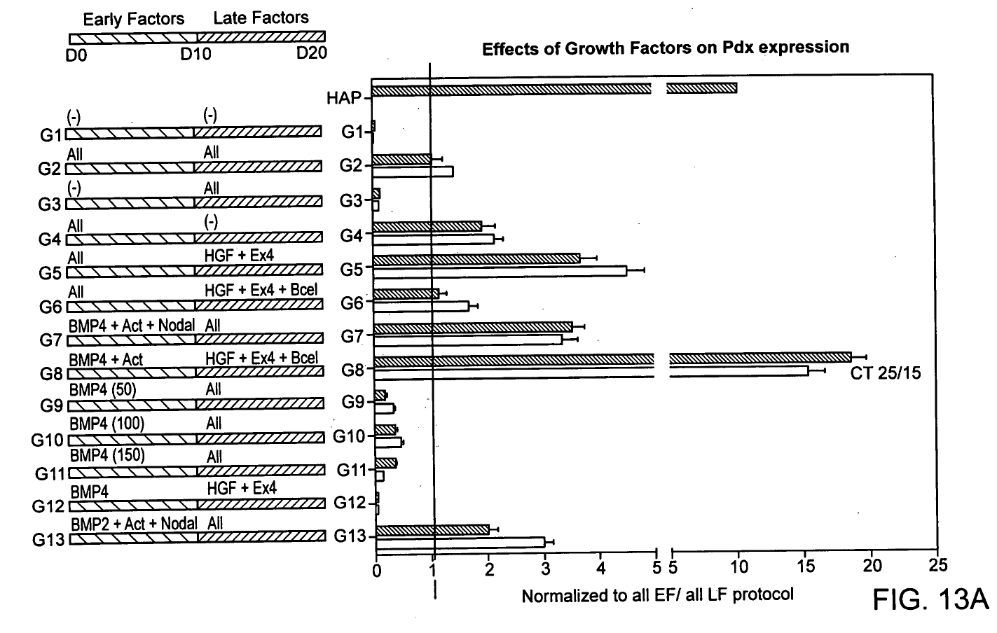

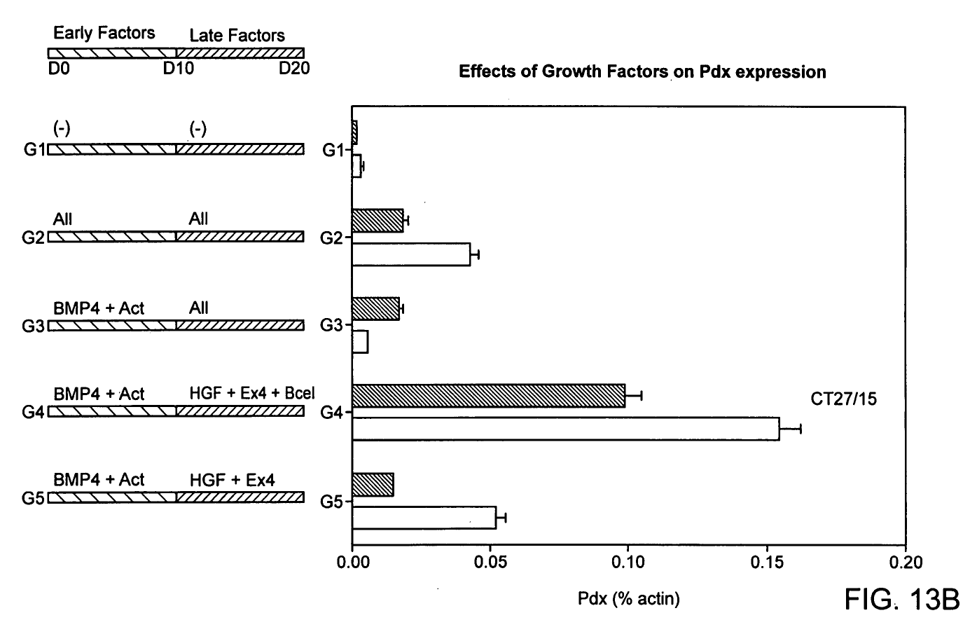

FIGS. 13A and 13B summarize the results of experiments designed to examine the effect on induction of pdx-1 expression of different combinations of early and late factors. Note that the results depicted in FIG. 13A represent normalized expression, and the results depicted in FIG. 13B represent expression as % of actin input.

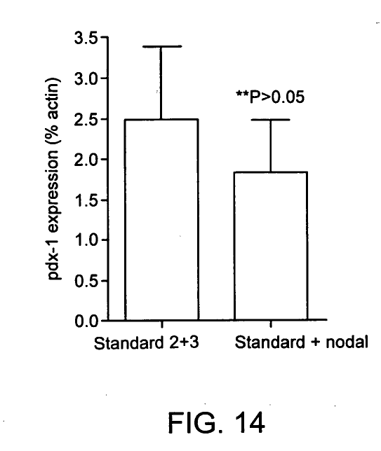

FIG. 14 shows the effect of nodal on induction of pdx-1 expression. The 2 EF-3 LF protocol was performed in the presence or absence of 50 ng/ml of recombinant nodal protein.

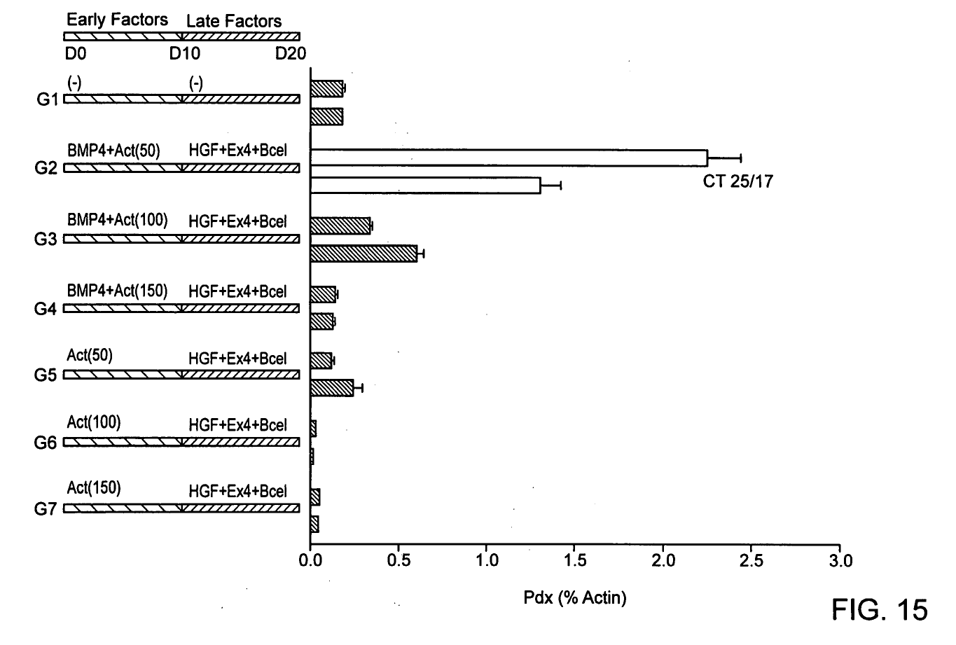

FIG. 15 shows the effect of activin and BMP4 protein concentrations on induction of pdx-1 expression. The data were determined by pdx-1 and actin standard curves, and are expressed as % actin.

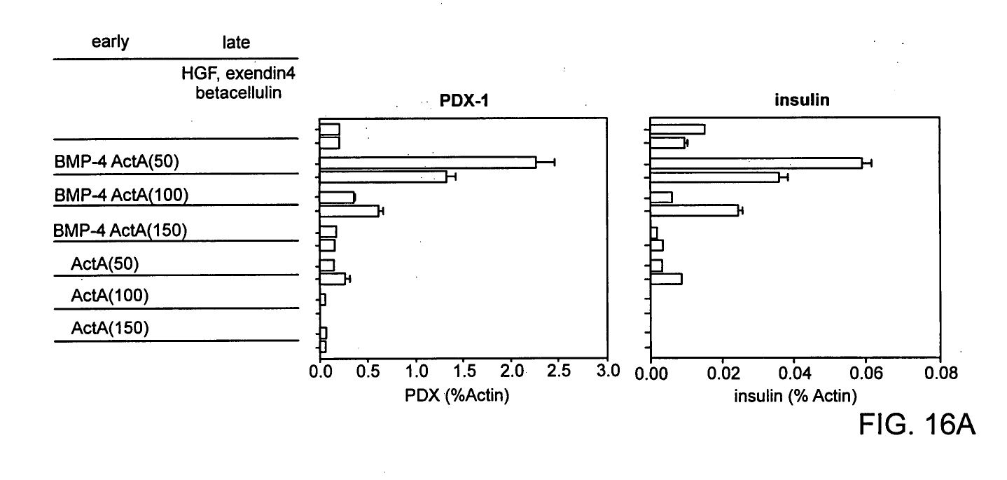

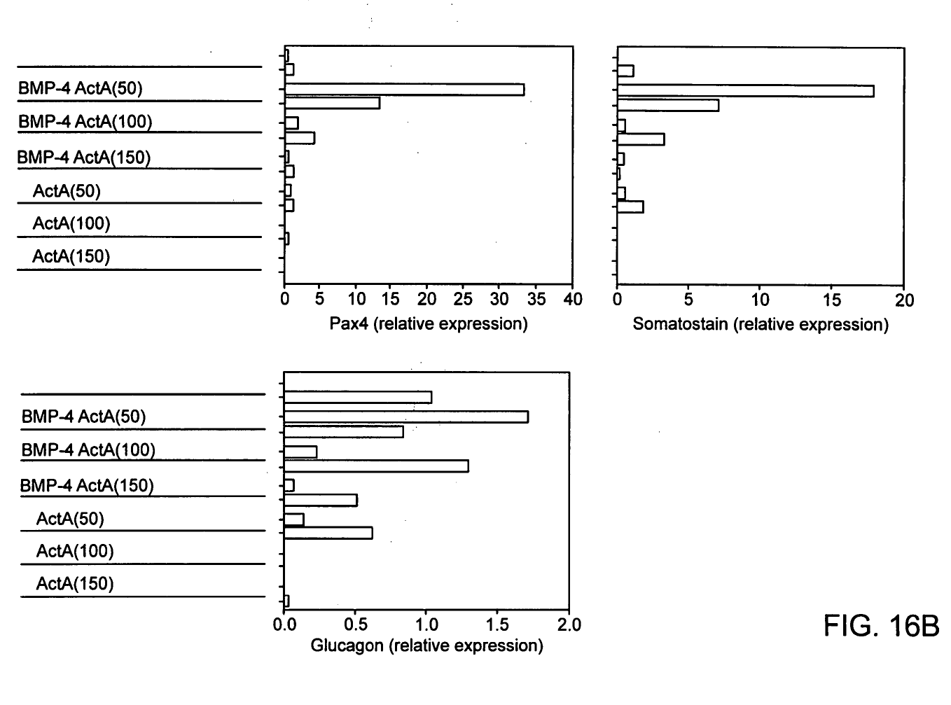

FIG. 16 shows the effect of activin and BMP4 protein concentrations on induction of expression of a number of endocrine genes. Pdx-1 and insulin gene expression were calculated based on standard curves and expressed as % actin. Pax4, somatostatin, and glucagon were calculated as relative values.

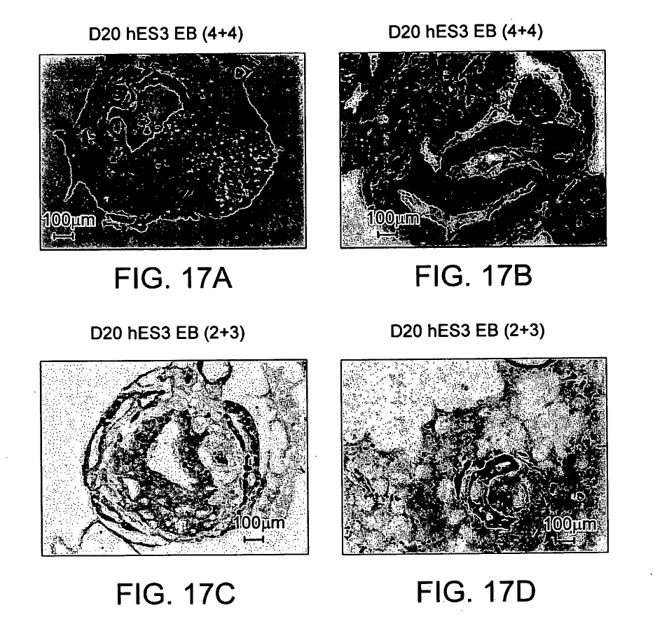

FIGS. 17A-17D show that the 2 EF-3 LF initial differentiation protocol (panels C and D) more effectively induces pdx-1 expression than the 4 EF-4 LF initial differentiation protocol (panels A and B).

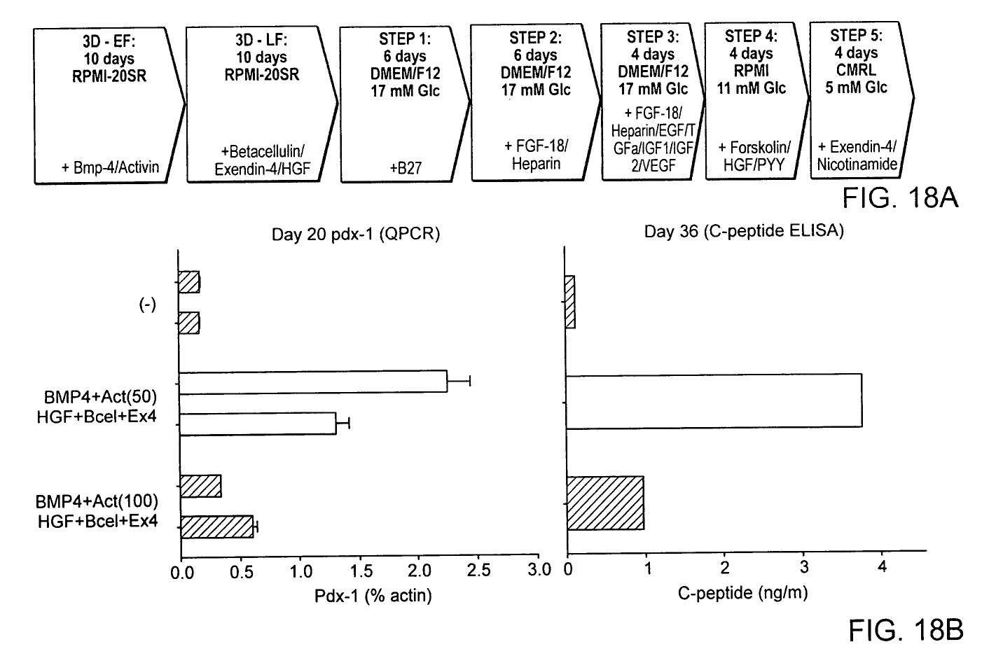

FIGS. 18A and 18B show pdx-1 expression followed by release of C-peptide from representative cultures of embryonic stem cells differentiated using an extended differentiation protocol including both an initial differentiation phase and a maturation phase. FIG. 18A is a schematic representation of the combined protocol. The left panel of FIG. 18B shows the expression of pdx-1 by quantitative PCR following the first 20 days of differentiation (the initial differentiation protocol). The right panel of FIG. 18B shows release of C-peptide at day 36 of differentiation. Day 36 is approximately half way through the maturation portion of the extended differentiation protocol.

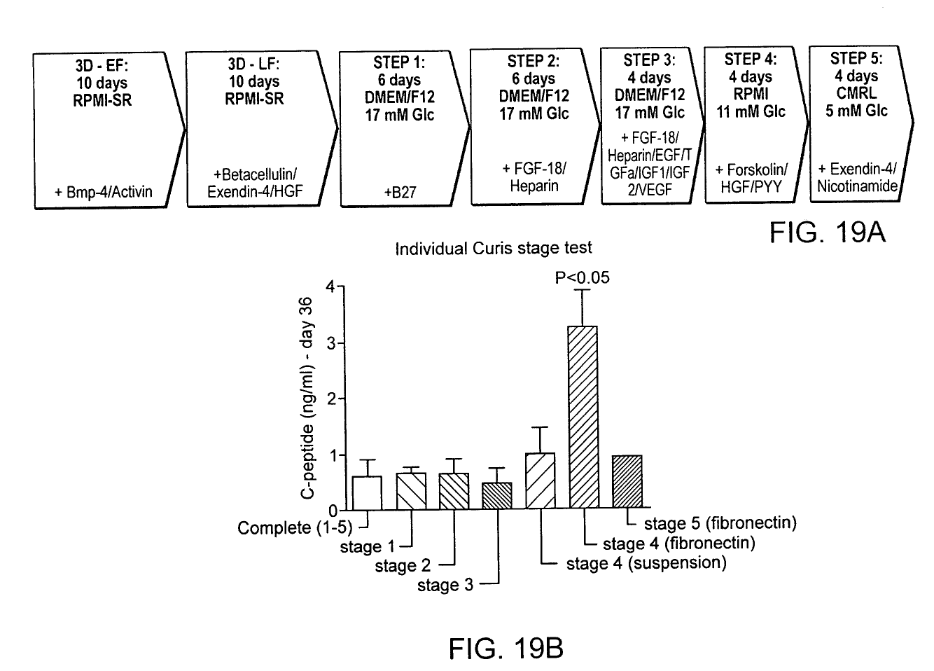

FIGS. 19A and 19B show release of C-peptide from cultures assayed at various stages during the extended differentiation protocol.

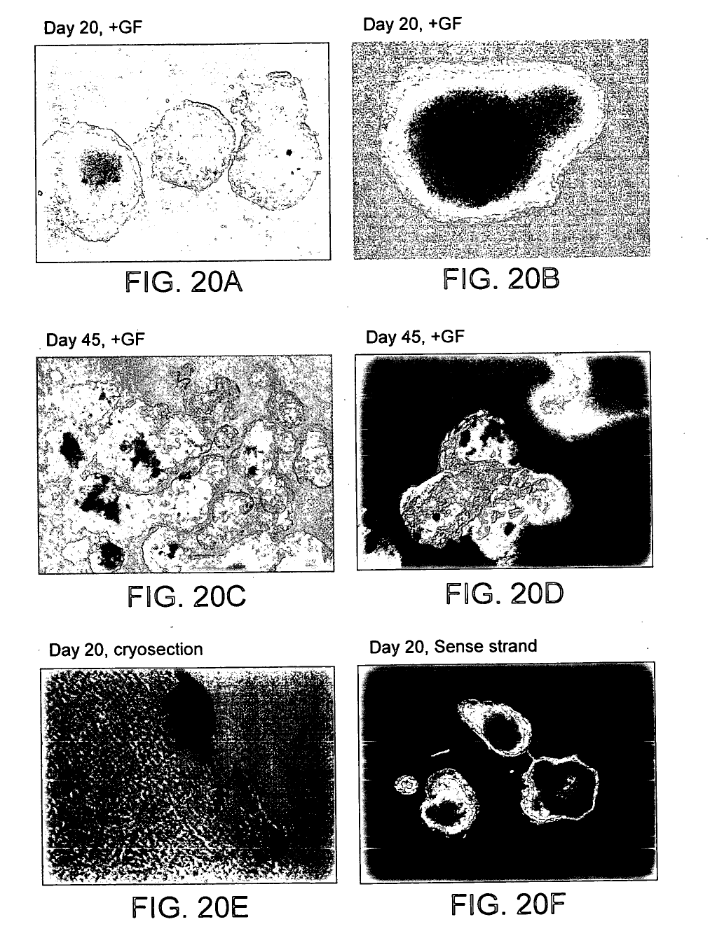

FIGS. 20A-20F show insulin expression by in situ hybridization. FIGS. 20A and 20B show that after 20 days in the initial differentiation protocol, embryoid bodies contain a few isolated insulin⁺ cells. FIGS. 20C and 20D show that further differentiation using the maturation protocol induces insulin expression in a higher percentage of cells in the embryoid body. Additionally, following the maturation protocol, the insulin⁺ cells appear most prevalent within sectors/clusters within the embryoid body. FIG. 20E shows a cryosection of a day 20 embryoid body. FIG. 20F shows a sense strand negative control.

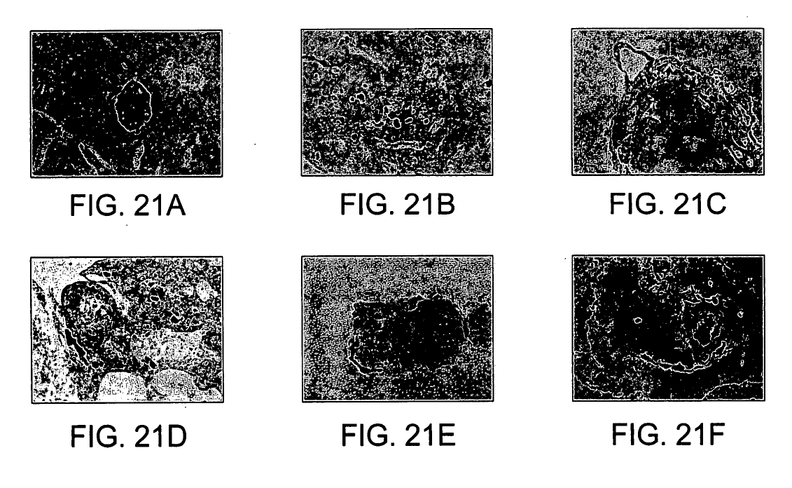

FIGS. 21A-21F show C-peptide protein expression by immunocytochemistry in day 45 embryoid bodies.

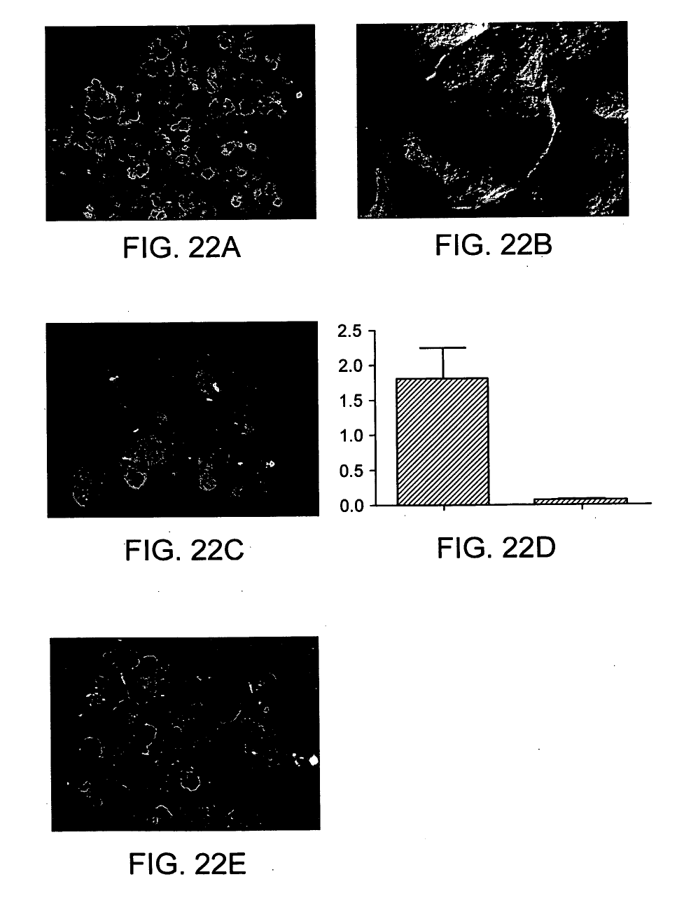

FIGS. 22A-22E show pdx-1 expression by in situ hybridization. FIGS. 22A and 22B show pdx-1 expression in embryoid bodies cultured in the initial differentiation protocol for 20 days. FIG. 22C show that embryoid bodies cultured for 20 days in the absence of growth factors fail to express pdx-1. FIG. 22D summarizes the results of the experiments depicted in FIGS. 22A and 22C, and confirms robust pdx-1 expression in cells cultured for 20 days in the presence (left) versus the absence (right) of growth factors. FIG. 22E shows that after 43 days in a combination of the initial and maturation protocols, embryoid bodies robustly express pdx-1. Pdx-1 expression is generally clustered to a portion of a particular embryoid body.

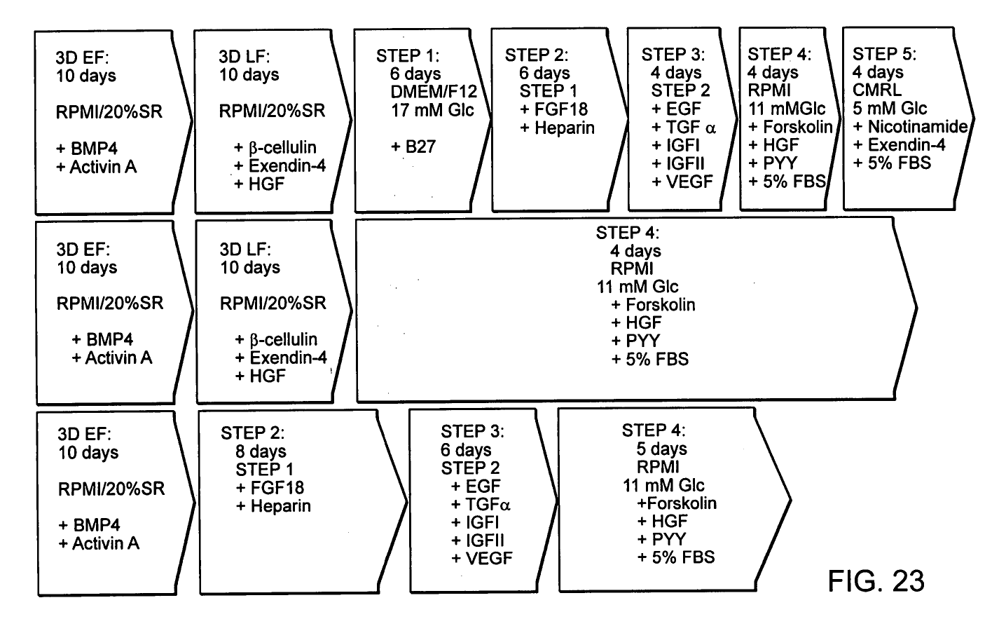

FIG. 23 shows two variations of the multi-step maturation protocol that result in C-peptide release. The top diagram is identical to that shown in FIG. 19. The middle and bottom diagrams show two variations, each more efficient than the top diagram protocol.

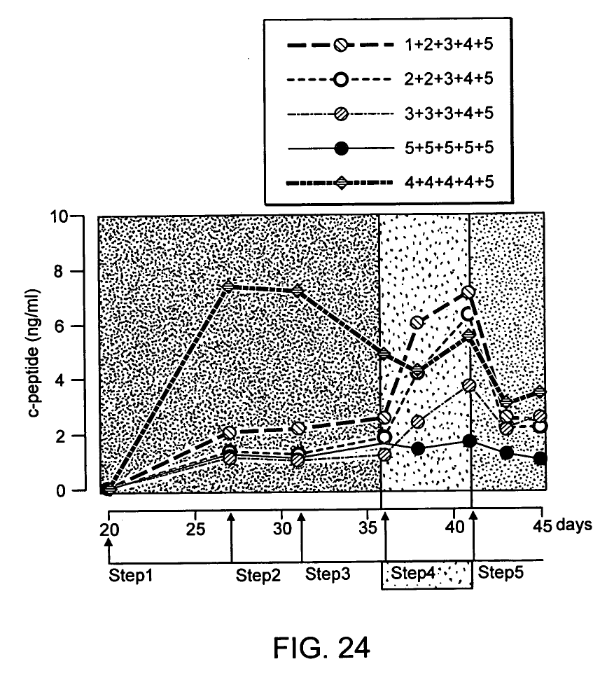

FIG. 24 shows the release of C-peptide when variations of the multi-step protocol was used.

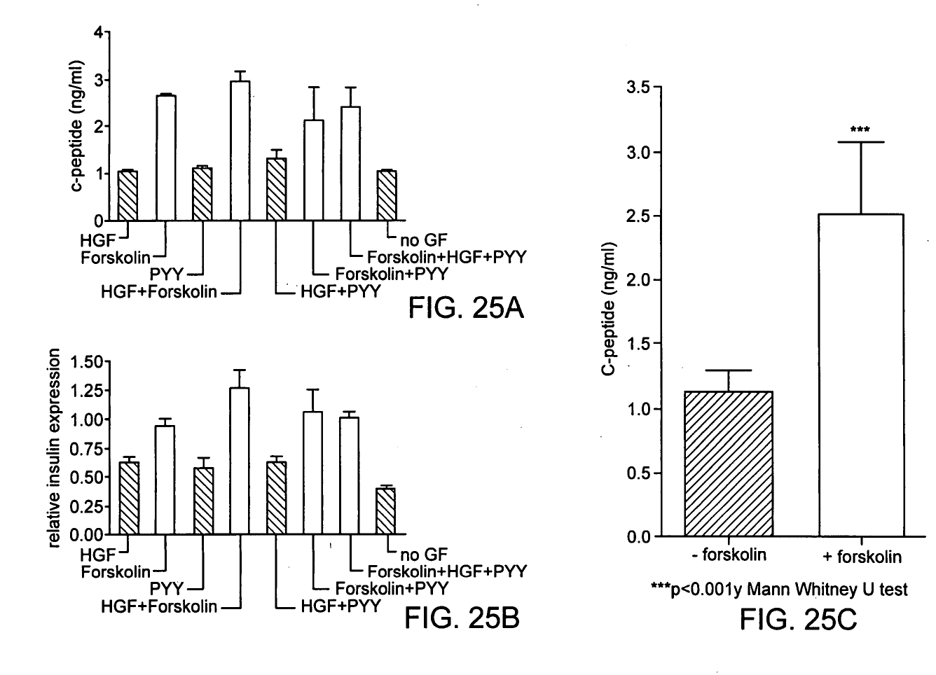

FIGS. 25A-25C show the effect of forskolin in Step 4 of the multi-step maturation protocol on the release of C-peptide.

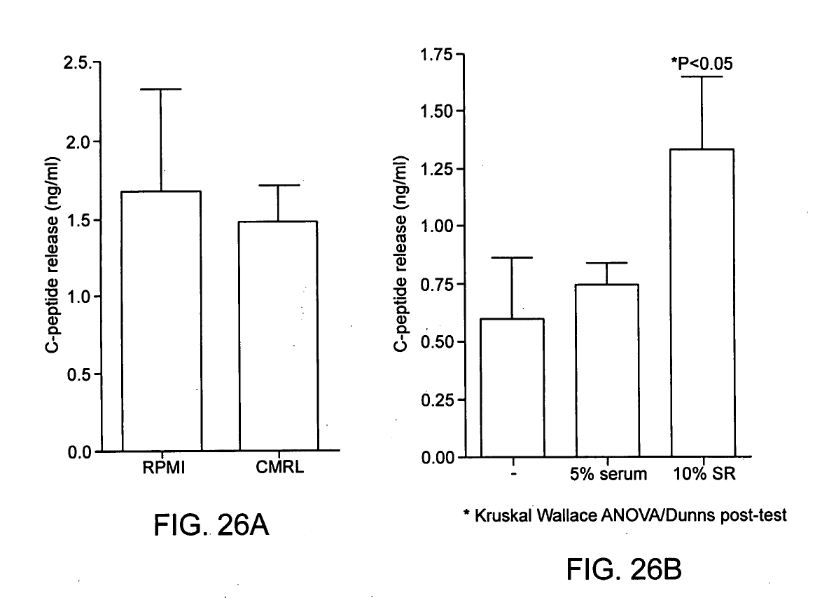

FIGS. 26A and 26B show the effect of fetal bovine serum (FBS) in Step 4 of the multi-step protocol on the release of C-peptide.

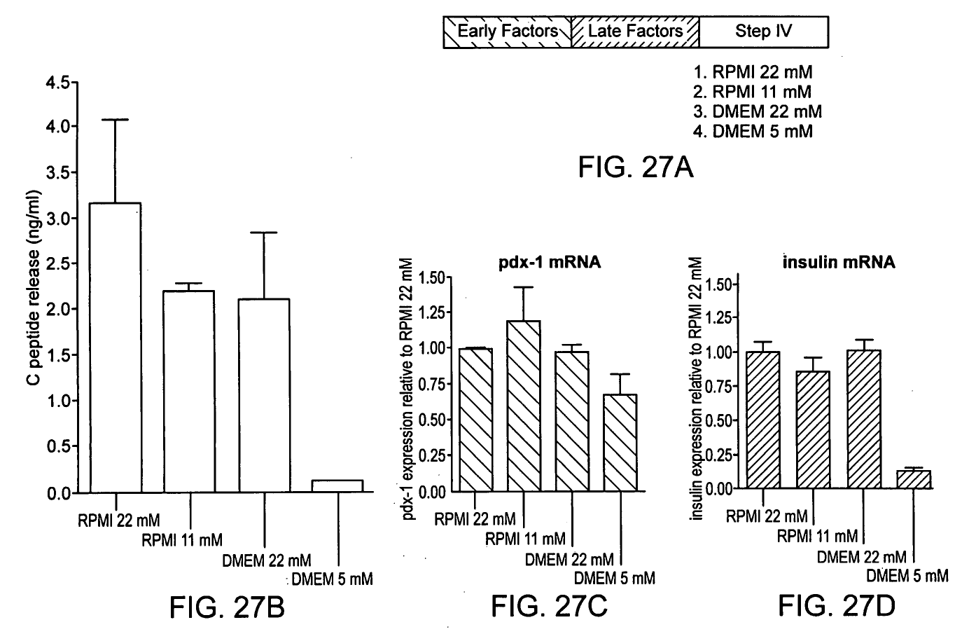

FIGS. 27A-27D show the protocol used (FIG. 27A), the effect of glucose concentration on differentiated HES3 cells measured by the release of C-peptide (FIG. 27B), pdx-1 mRNA (FIG. 27C) and insulin mRNA (FIG. 27D).

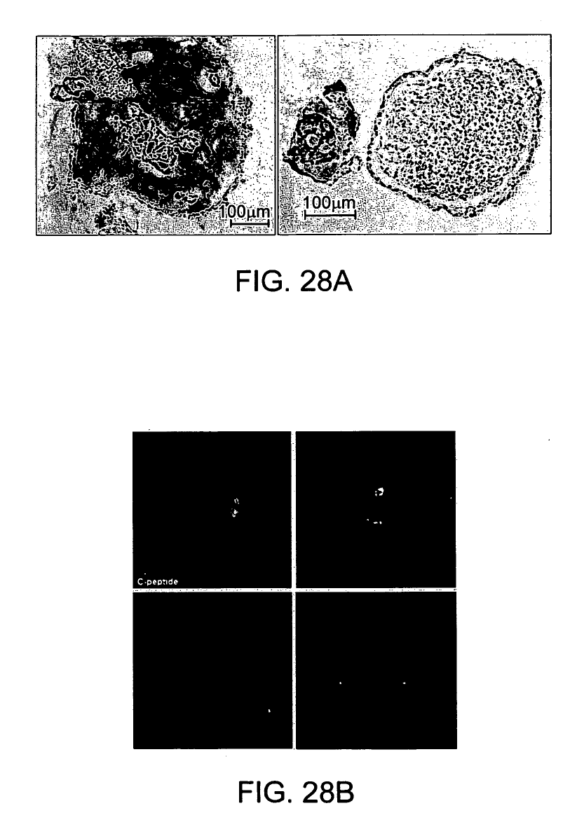

FIG. 28 shows the expression of Pdx-1 and C-peptide by single and double immunohistochemistry in differentiated HES3 embryoid bodies.

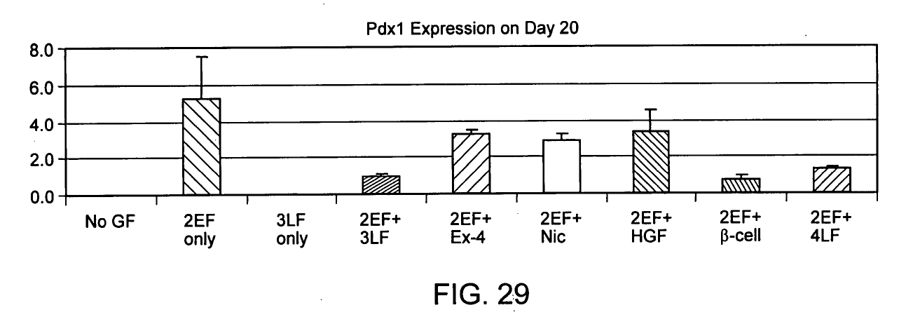

FIG. 29 shows the expression of Pdx-1 on Day 20 of MATRIGEL™ differentiation in the presence and absence of growth factors, various late factors and early factors.

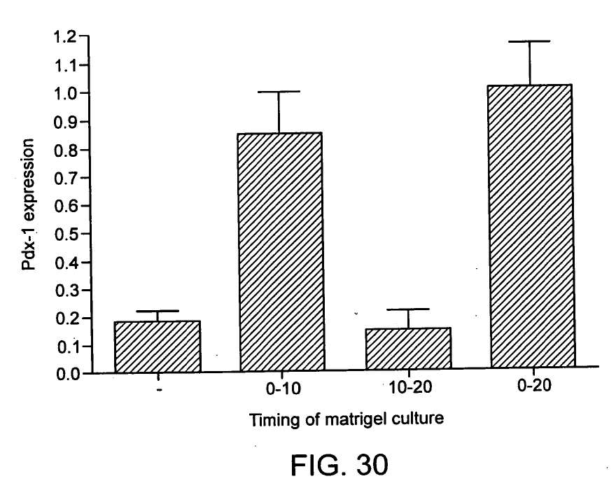

FIG. 30 shows the expression of Pdx-1 in the presence and absence of MATRIGEL™ between Day 0 and Day 10.

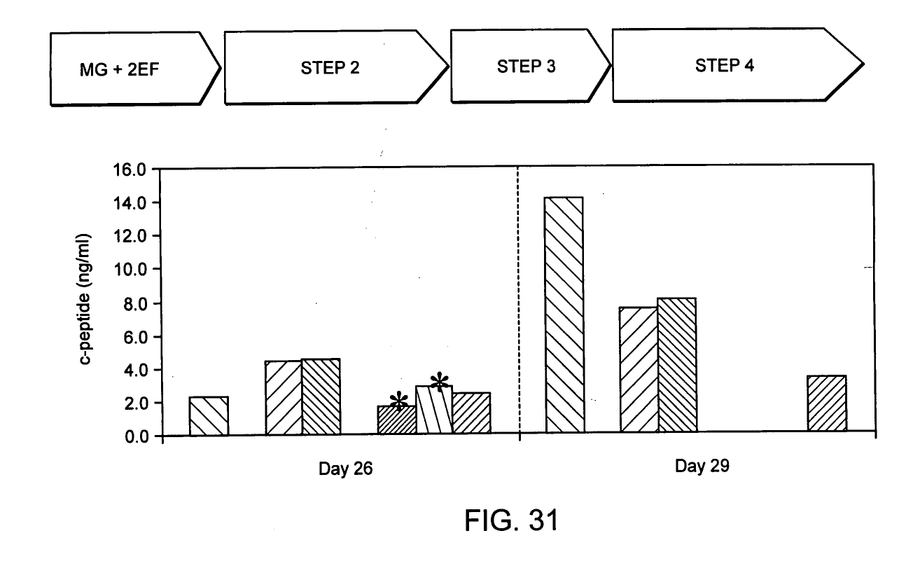

FIG. 31 shows the release of C-peptide on day 26 and day 29 when a simplified multi-step maturation protocol was used.

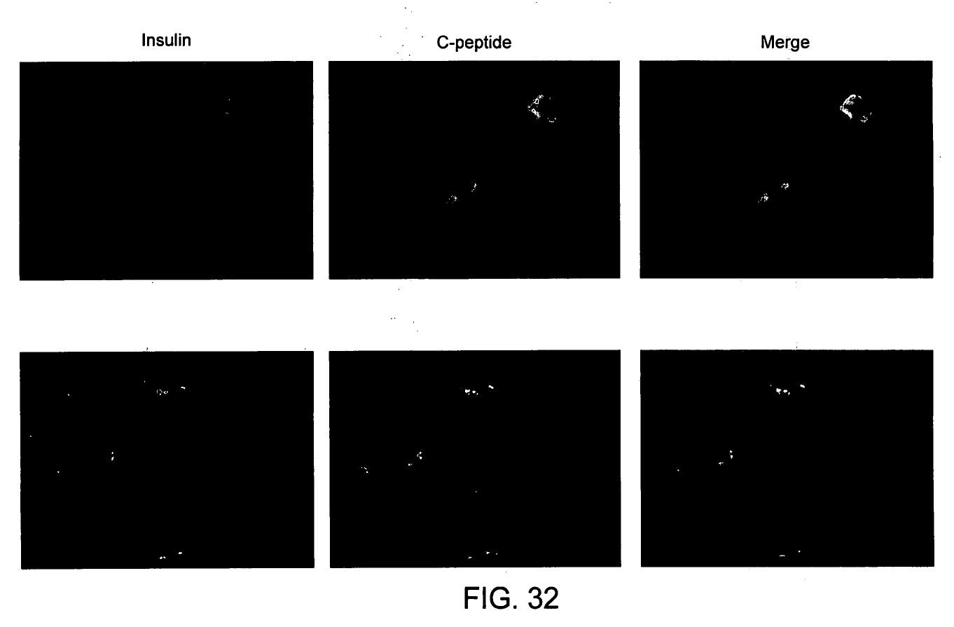

FIG. 32 shows the presence of insulin and C-peptide by double immunofluorescence in sectioned embryoid bodies. The top panels are high magnification images and the bottom panels are low magnification images.

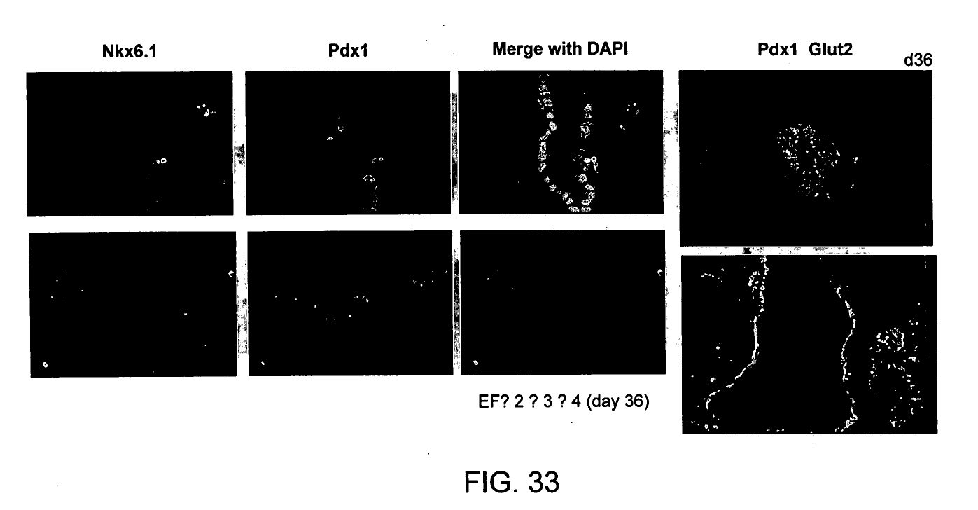

FIG. 33 shows the expression of Pdx-1 and Nkx6.1, both differentiation markers of β-cell endocrine lineage.

### DETAILED DESCRIPTION OF THE INVENTION

(i) Overview

Diabetes mellitus is a common disease characterized by the inability to regulate circulating glucose levels due to problems with insulin production or utilization. Type I diabetes (about 5% of all diabetes cases) is caused by the autoimmune destruction of the pancreatic beta cell that produces insulin. The more common type 2 diabetes, associated with obesity, has many causes related to either a decreased insulin output by the pancreas or to inefficient utilization of insulin at the target organs (insulin resistance). Collectively, diabetes can be considered a global epidemic, affecting as many as 7.9% of the American people. The very nature of the diabetic pathology, namely the autoimmune destruction or the decreased efficiency of the pancreatic beta cell, makes it an ideal candidate for cell therapy. Recent breakthroughs in islet transplantation that draw upon improved islet isolation techniques and immunosuppression regimes have been very successful at keeping patients free from insulin dependency for extended periods of time. However, the limited supply of cadaver pancreatic tissue makes this approach inadequate to meet the global patient demand for treatment. Researchers are therefore focusing on locating other sources of beta cells, of which embryonic stem cells are an attractive choice.

Several labs have reported the differentiation of insulin-producing cells from mouse ES cells. The protocols from the McKay lab depend on the isolation and purification of cells that are nestin-positive and is based on the assumption that beta cells might first pass through a neuronal intermediate. However, the insulin release observed in this article is likely caused by insulin uptake from the cell culture media. Furthermore, several labs have demonstrated that morphologically, the cells produced by these protocols are quite different from a bone fide beta cell, and in fact are more neuron-like with the acquired capacity for insulin uptake and release.

The work in human ES cells has lagged behind that in mouse, due in large part to the infancy of the human ES field in general and international legislature against public hESC research. The published reports detailing beta cell differentiation from mouse ES cells are worthy pioneering efforts, and have fostered optimism for the field, yet are menaced by reports of irreproducibility or poor efficiency. This leaves a huge opening in the field of human ES cells to develop a robust directed differentiation protocol that efficiently produces large numbers of beta cells for eventual cell therapy.

Both the rise in cases of diabetes and the recent success of the Edmonton protocol as a method of treating diabetes have placed great optimism on cell therapeutic methods to cure the disease. Though a highly competitive field, there are not yet any efficient, reproducible protocols available to direct differentiation of pluripotent human embryonic stem cells (hESCs) towards a pancreatic beta cell-like phenotype. The present invention provides a variety of methods to direct the differentiation of embryonic stem cells to a pancreatic cell fate. These include methods comprising the use of several early and late factors (EF and LF) administered over an approximately 20 day time frame. This initial differentiation methodology promotes expression of pdx-1 and promotes differentiation of embryonic stem cells along a pancreatic lineage. Additionally, this initial differentiation methodology promotes expression of markers of terminal pancreatic differentiation, such as insulin and somatostatin, though at a lower level than that of pdx-1. The present invention provides a variety of experiments identifying factors and optimized sub-sets of early and late factors that help promote differentiation of embryonic stem cells along a pancreatic lineage.

In addition to a variety of initial differentiation methods, the present invention provides maturation protocols designed to further promote the differentiation of stem cells along a pancreatic lineage. Specifically, the invention provides maturation protocols that can be used to promote terminal differentiation of embryonic stem cells that were previously directed along a pancreatic lineage using the initial differentiation protocols detailed herein. Using the maturation protocols, embryonic stem cells can be further differentiated to induce and/or increase expression of terminal differentiation markers including, but not limited to, insulin and C-peptide. Furthermore, such maturation protocols can be used to produce cell or cell clusters that are glucose responsive (e.g., mimic a function of pancreatic beta cells).

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

“Homology” and “identity” are used synonymously throughout and refer to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

As used herein, “small organic molecule” refers to compounds smaller than proteins that are generally characterized by the ability to transit cellular membranes more easily than proteins. Preferred small organic molecules are characterized as having a size less than 10,000 AMU. More preferably, between 5000-10,000 AMU. Most preferably, the small organic molecules are characterized as having a size between 1000-5000 AMU.

The “non-human animals” of the invention include mammals such as rats, mice, rabbits, sheep, cats, dogs, cows, pigs, and non-human primates.

As used herein, “proliferating” and “proliferation” refer to cells undergoing mitosis.

“Differentiation” in the present context means the formation of cells expressing markers known to be associated with cells that are more specialized and closer to becoming terminally differentiated cells incapable of further division or differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “tissue” refers to a group or layer of similarly specialized cells which together perform certain special functions.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are either undifferentiated, are differentiated to a non-endodermal cell type, or are differentiated to an endodermal tissue type that is not functionally or structurally related to that of the essentially purified population of cells.

A “marker” is used to determine the state of a cell. Markers are characteristics, whether morphological or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope. The invention contemplates methods of analyzing the progressive or terminal differentiation of a cell employing a single marker, as well as any combination of molecular and/or non-molecular markers.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that the term “differentiation” or “differentiated” refers to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment. Cells which have become more specialized but are not yet terminally differentiated are referred to as partially differentiated.

The terms “initiation protocol” or “initiation method” are used interchangeably to refer to any of the various methods of the invention used to begin biasing embryonic stem cells and embryoid bodies along a pancreatic lineage. The initiation protocol is typically approximately 20 days and includes addition of early factors (EF) and late factors (LF). However, initiation protocols of shorter durations, for example 10 days in the presence of only EFs, are also contemplates Exemplary initiation protocols include, but are not limited to, the eight factor protocol comprising addition of 4 EFs and 4 LFs, as well as the 2 EF-3 LF protocol. Throughout the application particular initiation protocols are also referred to more specifically according to the number or combination of early and late factors used to help promote initial differentiation of embryonic stem cells along a pancreatic lineage.

The term “maturation protocol” is used to refer to any of the various methods used to further differentiate embryonic stem cells and embryoid bodies previous subjected to the initiation protocol. The maturation protocol can be subdivided into various stages, and the term maturation protocol will be used to refer to methods where the cells are subjected to any or all of these various phases. Specific reference to the number of days in culture, the stage of the protocol, or the factors added will be used to help distinguish the various permutations and stages of the maturation protocol(s).

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The terms “hedgehog signaling,” “hedgehog signal transduction,” and “hedgehog signaling pathway” are used interchangeably throughout the application to refer to the mechanism whereby hedgehog proteins (Sonic, Desert, Indian hedgehog) influence proliferation, differentiation, migration, and survival of diverse cell types (see, for example, Allendoerfer (2003) Current Opinion Investig. Drugs 3: 1742-1744; Ingham (2001) Genes & Dev 15: 3059-3087). Agents that promote hedgehog signal transduction are referred to as “hedgehog agonists” or “agonists of hedgehog signaling.” Agents that inhibit hedgehog signal transduction are referred to as “hedgehog antagonists” or “antagonists of hedgehog signaling.” Hedgehog signal transduction may be influenced by hedgehog proteins, or by agents that agonize or antagonize hedgehog signaling at any point in the pathway (extracellularly, at the cell surface, or intracellularly). For further examples see U.S. Pat. No. 6,444,793; U.S. Pat. No. 6,683,108; U.S. Pat. No. 6,683,198; U.S. Pat. No. 6,686,388; WO 02/30421; WO 02/30462; WO 03/011219; WO 03/027234; WO 04/020599. Each of the foregoing references are hereby incorporated by reference in their entirety.

The terms “BMP signaling,” “BMP signal transduction,” and “BMP signaling pathway” are used interchangeably throughout the application to refer to the mechanism whereby BMP proteins influence proliferation, differentiation, migration, and survival of diverse cell types (see, for example, Balemans (2002) Developmental Biology 250: 231-250; U.S. Pat. No. 6,498,142; Miyazawa et al. (2002) Genes Cell 7: 1191-1204). Agents that promote BMP signal transduction are referred to as “BMP agonists” or “agonists of BMP signaling.” Agents that inhibit BMP signal transduction are referred to as “BMP antagonists” or “antagonists of BMP signaling.” BMP signal transduction may be influenced by BMP proteins, or by agents that agonize or antagonize BMP signaling at any point in the pathway (extracellularly, at the cell surface, or intracellularly).

The terms “Wnt signaling,” “Wnt signal transduction,” and “Wnt signaling pathway” are used interchangeably throughout the application to refer to the mechanism whereby Wnt proteins influence proliferation, differentiation, migration, and survival of diverse cell types (see, for example, WO 02/44378; Wharton, Developmental Biology 253: 1-17, 2003). Agents that promote Wnt signal transduction are referred to as “Wnt agonists” or “agonists of Wnt signaling.” Agents that inhibit Wnt signal transduction are referred to as “Wnt antagonists” or “antagonists of Wnt signaling.” Wnt signal transduction may be influenced by Wnt proteins, or by agents that agonize or antagonize Wnt signal transduction at any point in the pathway (extracellularly, at the cell surface, or intracellularly).

The terms “Notch signaling,” “Notch signal transduction,” and “Notch signaling pathway” are used interchangeably throughout the application to refer to the mechanism whereby Notch proteins influence proliferation, differentiation, migration, and survival of diverse cell types (see, for example, Baron, Stem Cell Dev. Bio. 14: 113-119, 2003). Agents that promote Notch signal transduction are referred to as “Notch agonists” or “agonists of Notch signaling.” Agents that inhibit Notch signal transduction are referred to as “Notch antagonists” or “antagonists of Notch signaling.” Notch signal transduction may be influenced by a Notch proteins, or by agents that agonize or antagonize Notch signal transduction at any point in the pathway (extracellularly, at the cell surface, or intracellularly).

The term “adherent matrix” refers to any matrix that promotes adherence of cells in culture (e.g., fibronectin, collagen, laminins, superfibronectin). Exemplary matrices include MATRIGEL™ (Beckton-Dickinson), HTB9 matrix, and superfibronectin. MATRIGEL™ is derived from a mouse sarcoma cell line. HTB9 is derived from a bladder cell carcinoma line (U.S. Pat. No. 5,874,306).

The term “pancreas” is art recognized, and refers generally to a large, elongated, racemose gland situated transversely behind the stomach, between the spleen and duodenum. The pancreatic exocrine function, e.g., external secretion, provides a source of digestive enzymes. Indeed, “pancreatin” refers to a substance from the pancreas containing enzymes, principally amylase, protease, and lipase, which substance is used as a digestive aid. The exocrine portion is composed of several serous cells surrounding a lumen. These cells synthesize and secrete digestive enzymes such as trypsinogen, chymotrypsinogen, carboxypeptidase, ribonuclease, deoxyribonuclease, triacylglycerol lipase, phospholipase A₂, elastase, and amylase.

The endocrine portion of the pancreas is composed of the islets of Langerhans. The islets of Langerhans appear as rounded clusters of cells embedded within the exocrine pancreas. Four different types of cells-α, β, δ, and φ-have been identified in the islets. The α cells constitute about 20% of the cells found in pancreatic islets and produce the hormone glucagon. Glucagon acts on several tissues to make energy available in the intervals between feeding. In the liver, glucagon causes breakdown of glycogen and promotes gluconeogenesis from amino acid precursors. The δ cells produce somatostatin which acts in the pancreas to inhibit glucagon release and to decrease pancreatic exocrine secretion. The hormone pancreatic polypeptide (PP) is produced in the φ cells. This hormone inhibits pancreatic exocrine secretion of bicarbonate and enzymes, causes relaxation of the gallbladder, and decreases bile secretion. The most abundant cell in the islets, constituting 60-80% of the cells, is the β cell, which produces insulin. Insulin is known to cause the storage of excess nutrients arising during and shortly after feeding. The major target organs for insulin are the liver, muscle, and fat-organs specialized for storage of energy.

The term “pancreatic duct” includes the accessory pancreatic duct, dorsal pancreatic duct, main pancreatic duct and ventral pancreatic duct. Serous glands have extensions of the lumen between adjacent secretory cells, and these are called intercellular canaliculi. The term “interlobular ducts” refers to intercalated ducts and striated ducts found within lobules of secretory units in the pancreas. The “intercalated ducts” refers to the first duct segment draining a secretory acinus or tubule. Intercalated ducts often have carbonic anhydrase activity, such that bicarbonate ion may be added to the secretions at this level. “Striated ducts” are the largest of the intralobular duct components and are capable of modifying the ionic composition of secretions.

As used herein, “islet equivalents” or “IEs” is a measure used to compare total insulin content across a population or cluster of cells. An islet equivalent is defined based on total insulin content and an estimate of cell number which is typically quantified as total protein content. This allows standardization of the measure of insulin content based on the total number of cells within a cell cluster, culture, sphere, or other population of cells. The standard rat and human islet is approximately 150 μm in diameter and contains 40-60 ng insulin/μg of total protein. On average, human islet-like structures differentiated by the methods of the present invention contain approximately 50 ng insulin/μg of total protein.

The term “reporter construct” is used to refer to constructs that ‘report’ or ‘identify’ the presence of particular cells. Typically reporter constructs include portions of the promoter, enhancer, or other regulatory sequences of a particular gene sufficient to regulate expression in a developmentally relevant manner. Such regulatory sequences are operably linked to a nucleic acid sequence encoding a marker that can be readily detectable (the ‘reporter gene’). In this way, expression of a readily detectable product can be monitored, and this product is regulated in a manner consistent with the promoter or enhancer to which it is operably linked. Reporter genes may be introduced into cells by any of a number of ways including transfection, electroporation, micro-injection, etc. Exemplary reporter genes include, but are not limited to, green fluorescent protein (GFP), recombinantly engineered variants of GFP, red fluorescent protein, yellow fluorescent protein, cyan fluorescent protein, LacZ, luciferase, firefly Remmila protein. Further exemplary reporter genes encode antibiotic resistance proteins including, but not limited to, neomycin, hygromycin, zeocine, and puromycin.

The term “xeno-free/clinically compliant ES cells or cell lines” or “xeno-free CGMP-compliant hES lines” refers to

All current 78 U.S. National Institutes of Health (NIH)-listed human embryonic stem cell (hESC) lines approved for U.S. government federal research funding have been derived and propagated on mouse embryonic fibroblasts (MEFs) and in the presence of culture medium containing animal-based ingredients. The use of a feeder layer of animal origin and animal components in the culture media may potentially substantially elevates the risk of the cross-transfer of viruses and other pathogens to the embryonic stem (ES) cells. Hence, safer current good manufacturing practice (CGMP) and good tissue culture practice (GTCP)-compliant hESC lines and differentiated hESC progenitors are more suitable for clinical application.

Several attempts at improving hESC culture conditions have been reported. These advances include the use of conditioned media together with MATRIGEL™ as an attachment substrate for hESC culture, and the derivation and propagation of hESC lines on human feeder layers. These improvements are important steps forward in developing a CGMP-compliant protocol for the establishment of xeno-free clinically compliant hESC lines. The derivation of xeno-free CGMP-compliant hES lines also necessitates the development of a cryopreservation protocol that is effective and minimizes or restricts the possibility of cell line contamination in long-term liquid nitrogen (LN2) storage. At least two freezing protocols are currently used for hESCs. These include (a) the conventional slow stepwise programmed freezing method using cryovials (CVs) and storage in LN2 and (b) a snap-freezing vitrification method using an open pulled straw (OPS) and storage in LN2. Another effective, safe, and sterile cryopreservation protocol is described by Richards et al. (Stem Cells 22: 779-789, 2004). These protocols can be used for generation and long-term storage of CGMP- and GTCP-compliant xeno-free hESC lines useful for the instant invention.

(iii) Exemplary Methods

Methods of isolating and maintaining undifferentiated cultures of embryonic stem cells from any of a variety of species are well known in the art. Exemplary species include, but are not limited to, mice, non-human primates, and humans. Furthermore, under a variety of circumstances, many have observed the differentiation of embryonic stem cells to any of a number of partially or terminally differentiated cell types. For example, embryonic stem cells aggregated to form embryoid bodies may produce embryoid bodies that include small regions or foci of beating tissue. This beating tissue indicates that a small percentage of cells in the embryoid body have differentiated to form cardiomyocytes.

However, the challenge is not to wait patiently as embryonic stem cells randomly differentiate along particular lineages. Nor is the challenge to devise methods of differentiating embryonic stem cells that produce a disparate “mixed bag” of cell types across a culture. At this point, the challenge is to develop efficient methods to direct the differentiation of embryonic stem cells to particular cell types or along particular developmental lineages. Such methods are essential to increase our understanding of stem cell biology, to produce substantially purified cultures of differentiated cell types, and to develop therapeutics based upon differentiated cells.

The present invention addresses the limitations in the prior art and offers methodologies for directing the differentiation of embryonic stem cells to endodermal cell types. Specifically, the methods of the present invention can be used to direct the differentiation of embryonic stem cells to produce various partially and/or terminally differentiated cells or cell clusters. By way of example, the methods of the present invention (e.g., the initiation protocols, the maturation protocols, and combinations thereof) can be used to direct the differentiation of embryonic stem cells to partially and terminally differentiated pancreatic cell types.

Partially and terminally differentiated cell types, for example pancreatic cell types, induced by the initiation and/or maturation protocols of the present invention can be further expanded and/or purified to produce essentially purified cultures of one or more partially and/or terminally differentiated endodermal cell types. By way of non-limiting example, the methods of the present invention can be used to produce, from embryonic stem cells, (i) essentially purified populations of terminally differentiated pancreatic cell types (e.g., either a single terminally differentiated pancreatic cell type or multiple terminally differentiated pancreatic cell types); (ii) essentially purified populations of partially differentiated pancreatic cell types (e.g., either a single partially differentiated pancreatic cell type or multiple partially differentiated pancreatic cell types); or (iii) essentially purified populations of one or more partially and/or terminally differentiated pancreatic cell types.

Cultures of embryonic stem cells (e.g., human, mouse, non-human primate, etc.) can be differentiated using methods that include a step involving formation of embryoid bodies or directly (e.g., without a step involving the formation of embryoid bodies). In one embodiment of the present invention, an early step in the differentiation process comprises the aggregation of embryonic stem cells to form embryoid bodies.

Differentiation Via Embryoid Body Formation

Embryonic stem (ES) cells can be differentiated by removing the cells from the feeder layer and aggregating them in suspension to form embryoid bodies (EBs). EBs can be made by plating dissociated ES cells in bulk on low-attachment plates or by the hanging drop method. ES cells may be dissociated fully into single cells or partially into small clumps by a number of methods including trypsin, collagenase, dispase, EDTA, or mechanical disruption. The method of dissociation can be readily selected by one of skill in the art and may vary depending on the species from which the cells are derived, as well as the overall health of the cells. For example, human ES cells do not survive as well following dissociation to the single cell level. Accordingly, when the methods of the present invention are performed using human ES cells, the dissociation technique can be selected so as to remove the ES cells from the feeder layer without dissociating the cells to the single cell level prior to EB formation. Following formation of EBs, the EBs can be cultured in suspension (e.g., as floating aggregates of cells, on filters, or embedded in gel-like matrices) for a period of time, preferably ranging from 3 days to 3 weeks. In certain embodiments, EBs can be cultured in suspension for less than 3 days, for example, for 6 hours, 12 hours, 18 hours, 24 hours, 36 hours, or 48 hours. In certain other embodiments, the EBs can be cultured for greater than 3 weeks.

Although general methods of aggregating ES cells to form EBs is known in the art, ES cells from certain species appear to be more sensitive to the level of dissociation achieved prior to EB formation. Accordingly, in addition to the above outlined approach in which certain ES cells are dissociated less completely (e.g., not dissociated to single cells) prior to EB formation, the present invention contemplates methods of EB formation in which ES cells are dissociated in the presence of agents that block apoptosis or otherwise promote cell survival. Exemplary agents include, but are not limited to, caspase inhibitors.

Following EB formation, EBs can be cultured in a variety of media including, but not limited to, basal media BME, CMRL1066, MEM, DMEM, DMEM/F12, RPMI, Glasgow MEM with or without alpha modification, IMDM, Leibovitz's L-15, McCoys 5A, Media 199, Ham's F-10, Ham's F-12, F-12K, NCTC-109 medium, Waymouth's media, William's Media E, or a combination of any of the above. One of skill in the art can readily select, based on cost, species, availability, etc., from amongst these and similar media designed for the culture of EBs. Any of the foregoing media can be supplemented with varying concentration of glucose (1-50 mM), sodium pyruvate, non-essential amino acids, nucleosides, N-2 supplement, G-5 supplement, and B27 supplement. The media may or may not contain phenol-red. Furthermore, the media can be buffered with an appropriate amount of buffering salt. Exemplary buffering salts include, but are not limited to, sodium bicarbonate, Tris, HEPES, sodium acetate. We note that the pH of the EB media can vary between 5 and 9.

In addition to the foregoing basal components of the EB media, in certain embodiments, the culture media may be supplemented with different amounts of animal serum. Exemplary animal sera commonly used in the art include, but are not limited to, fetal bovine serum (FBS), bovine serum (BS), horse serum (HS), chicken serum (CS), goat serum (GS). The serum may or may not be heat-activated. Alternatively, the media may be supplemented with a chemically defined serum replacement, such as Knockout DMEM with Knockout Serum Replacement. In one embodiment, the concentration of animal serum or serum replacement in the media is selected in the range from 0% to 20%. In other embodiments, the concentration of serum or serum replacement in the media is greater than 20%, for example, is between 20%-40%.

Although in certain embodiments, the EBs can be cultured in basal media supplemented with serum or serum replacement alone, EBs may also be cultured in media further containing media conditioned by another cell line. Alternatively, in another embodiment, EBs can be cultured in basal media lacking serum or serum replacement, but containing media conditioned by another cell line. Exemplary cell lines from which conditioned media can be obtained include, but are not limited to, mouse embryonic fibroblasts (MEFs); mouse or human insulinomas (e.g., RIN-5, beta-TC, NIT-1, INS-1, INS-2); hepatomas (e.g., HepG2, Huh-7, HepG3); HT-1080; endothelial cells (e.g., HUVEC); bone marrow stromal cells; visceral endoderm-like cells such as end-2; or mesenchymal cells such as HEPM or 7F2. Alternatively, conditioned media can be obtained from cultured embryonic, fetal, or adult tissues (e.g., derived from human, non-human primate, mouse, or other animals), or from a primary cell line established from a particular tissue type (e.g., pancreas, liver, bone marrow, lung, skin, blood, etc.) and derived from an animal (e.g., human, non-human primate, mouse, or other animal).

Embryonic tissues include endoderm, mesoderm, ectoderm, and/or extra-embryonic tissue such as trophectoderm and visceral endoderm. In certain embodiments employing conditioned media from embryonic tissue, the embryonic tissue is chosen based on the ability of that tissue to send instructive signals to the embryonic pancreas during development. Such instructive tissues include notochord and dorsal aorta. Exemplary primary cell lines include endothelial cells, aortic smooth muscle cells, mesenchymal cells, endocrine or exocrine cells of the pancreas, hepatocytes, intestinal epithelial cells, and ductal cells. Media can also be conditioned from any cells derived from mouse embryonic stem cells. In any of the foregoing embodiments in which EBs are cultured in the presence of conditioned media, the conditioned media may be derived from a cell line, tissue, etc. of the same species as the EBs or from a different species.

The foregoing media constitutes the starting point for directing the differentiation of the cells to a particular differentiated endodermal cell type. Any of the foregoing media can now be further supplemented with appropriate differentiation factors to direct cells in the EBs to differentiate along particular endodermal lineages such as the pancreatic lineage, hepatic lineage, lung lineage, etc. By way of example, EBs can be cultured in media supplemented with particular differentiation factors to direct the differentiation of cells in the EBs to pancreatic cell types including insulin-producing cells. Such factors include but are not limited to activin A, activin B, BMP2, BMP4, nodal, TGFβ, sonic hedgehog, desert hedgehog, EGF, HGF, FGF2, FGF4, FGF8, FGF18, PDGF, Wnt proteins, retinoic acid, sodium butyrate, NGF, HGF, GDF, growth hormone, PYY, cardiotropin, GLP-1, exendin-4, betacellulin, nicotinamide, tri-iodothyroxine, insulin, IGF-I, IGF-II, placental lactogen, VEGF, wortmannin, gastrin, cholecystokinin, Sphingosine-1-phosphate, FGF-10, FGF inhibitors, growth hormone, KGF, islet neogenesis-associated protein (INGAP), Reg, and factors that increase cAMP levels such as forskolin and IBMX. Most of the factors can be added into the media from a purified stock, or if they are protein factors, can be presented in the form of conditioned media taken from cells recombinantly expressing the factors. Additionally, the invention contemplates that, for certain of the above referenced protein factors, small molecule agonists that mimic the bioactivity of the protein are known in the art. Such small molecule mimics may function in any of a number of ways to produce similar biological consequences as the protein. Accordingly, the invention contemplates methods in which the EBs are cultured in the presence of small molecule agonists/mimics of any of the foregoing proteins.

Without being bound by theory, certain of these factors may influence cell fate by binding to receptors on the surface of the cells, and thereby modulating one or more signal transduction pathways functional in the cells. Alternatively, certain of these factors may influence cell fate by transiting the cell membrane and acting intracellularly to modulate one or more signal transduction pathways functional in the cells.

The invention contemplates using one or more of these factors to help promote the differentiation of cells to pancreatic cell types. In one embodiment, the one or more factors influence the cells by modulating the same signal transduction pathway (e.g., Sonic hedgehog protein in combination with Desert hedgehog protein). In another embodiment, the one or more factors influence the cells by modulating different signal transduction pathways (e.g., one or more hedgehog proteins in combination with one or more Wnt proteins). In another embodiment, one or more factors influence cells via mechanisms that may or may not overlap. Regardless of the precise mechanism of action, the invention contemplates that one or more of the above differentiation factors can be added to a culture of EBs to help promote their differentiation to pancreatic cell types. When more than one differentiation factor is added to the culture, the invention contemplates that the differentiation factors can be added concomitantly or concurrently.

The foregoing are exemplary of the factors and conditions that can be used to direct the differentiation of embryonic stem cells along particular lineages. By way of further specific example, the experiments summarized herein provide multiple examples of initiation protocols that bias embryonic stem cells along a pancreatic lineage. Furthermore, the experiments summarized herein provide multiple examples of maturation protocols that, when used in combination with an initiation protocol, help promote the further differentiation of biased embryonic stem cells to terminally differentiated pancreatic cell type (e.g., produce cells that express one or more markers indicative of a terminally differentiated pancreatic cell type).

Following a period of suspension as EBs in culture, which in one embodiment ranges from 3 days-3 weeks, the EBs can be replated on an adherent matrix. Exemplary adherent matrices include, but are not limited to, gelatin, MATRIGEL™, various types of collagens, laminins, fibronectins, or a combination of any of the foregoing. EBs can be replated directly onto the adherent matrix or EBs can be dissociated prior to replating onto the adherent matrix.

Directed Differentiation

In another embodiment, ES cells can be differentiated without a step including EB formation. For example, to initiate differentiation towards pancreatic cells types, ES cells can be plated directly on an appropriate adherent matrix without first forming cultures of EBs. The ES cells can also be differentiated either as a monolayer in culture or on feeder cells. The ES cells can be plated in any of the above referenced combination of media appropriate for the culture of EBs and further supplemented with one or more of the differentiation factors outlined above. Exemplary adherent matrices include, but are not limited to, gelatin, MATRIGEL™, various types of collagens, laminins, fibronectins, or a combination of any of the foregoing.

Regardless of whether the ES cells are differentiated directly or differentiated via EB formation, the invention contemplates that the ES cells, differentiating ES cells, or EBs can be cultured either under standard tissue culture conditions of oxygen and carbon dioxide, or in an incubator where oxygen tension can be varied.

In one embodiment of any of the foregoing, EBs can be cultured either in suspension in liquid media or in suspension by embedding in a 2D or 3D gel or matrix. Exemplary matrices include, but are not limited to, MATRIGEL™, collagen gel, laminin gel, as well as artificial 3D lattices constructed from materials such as polylactic acid or polyglycolic acid. When EBs are cultured suspended in a matrix, differentiation factors can be administered either by addition to the surrounding liquid medium or by covalently or non-covalently linking the factors to the particular matrix in which the EBs are suspended. In another embodiment, EBs can be cultured on a Transwell. Culture on a Transwell may facilitate establishment of cell polarity.

In one embodiment of any of the foregoing, differentiation of ES cells or EBs can be promoted by co-culturing the cells with cells, cell lines, or tissues of the endoderm or with cells, cell lines, or tissues derived from tissues known to induce endodermal differentiation during development.

Progressive Differentiation

In any of the foregoing methods of differentiation, the invention contemplates that a single differentiation step likely will not produce the particular partially or terminally differentiated cell type desired, or will not necessarily produce them in the desired ratios or percentages. Accordingly, the invention contemplates that ES cells and EBs can be cultured and differentiated in stages. At each successive stage, the differentiation factors and differentiation matrices may be the same or different.

Progressive differentiation of ES cells and EBs can be measured by examining markers of partially and/or terminally differentiated cells of the particular tissue of interest. For example, in methods where the goal is the differentiation of ES cells (with or without the formation of EBs) to pancreatic cell type, progressive differentiation can be monitored by assaying expression of markers of partially or terminally differentiated pancreatic cells (e.g., early markers of the endodermal lineage; early markers of the pancreatic lineage; markers of partially differentiated endocrine pancreatic cells; markers of partially differentiated exocrine pancreatic cells; markers of terminally differentiated endocrine pancreatic cells; markers of terminally differentiated exocrine pancreatic cells).

By way of example, early differentiation to definitive endoderm could be monitored by assaying expression of genes including, but not limited to, sox17, HNF1α, HNF3α, HNF3β, HNF3γ, brachyury T, goosecoid, claudins, AFP, HEX, eomesodermin, TCF2, Mix11, CXCR4, GATA5, and prox1. In contrast, differentiation into non-endodermal lineages could be monitored by assaying expression of non-endodermal genes, for example, genes indicative of extraembryonic tissue. Exemplary genes that could be used to assess the level of non-endodermal differentiation in a culture at a particular time include, but are not limited to, chorionic gonadotropin, amnionless, HNF4, GATA4, or GATA6.

As definitive endoderm is formed, partial or terminal differentiation towards the pancreatic lineage can be monitored by expression of pancreatic genes (e.g., exocrine pancreatic genes and endocrine pancreatic genes) including, but not limited to, pdx-1, ngn3, HB9, HNF6, ptf1-p48, islet 1, nkx6.1, nkx2.2, glut2, neuroD, cytokeratin 19, IAPP, pax4, pax6, HES1, amylase, glucagon, somatostatin, insulin, hormone convertase, glucokinase, Sur-1, Kirb6.2 and pancreatic polypeptide.

In any of the foregoing, gene expression can be measured in living cells over time to provide a snapshot of differentiation in a given culture. Alternatively, samples of cells from a given culture at a given time can be taken and processed. Such cells would provide a representation of the differentiation in a particular culture at a particular time.

Gene expression can be measured by a variety of techniques well known in the cell biological and molecular biological arts. These techniques include RT-PCR, northern blot analysis, in situ hybridization, microarray analysis, SAGE, or MPSS. Protein expression can similarly be analyzed using well known techniques such as western blot analysis, immunohistochemical staining, ELISA, or RIA.

Differentiation into definitive endoderm could be accomplished by activating certain pathways including but not limited to nodal, wnt, and FGF signaling. Nodal belongs to the TGF-beta superfamily of ligands that include activins and BMPs. Addition of these TGFβ related ligands could drive hES cells towards definitive endoderm. Wnt signaling could be activated by addition of any of the Wnt ligands. One main consequence of wnt signaling is the stabilization of β-catenin. Stabilization of β-catenin could also be accomplished by addition of GSK3 inhibitors, including but not limited to derivatives of 6-bromoindirubin. Inhibition of FGF signaling may also help to direct ES cells down the endodermal pathway. Inhibition of FGF signaling could be accomplished by using one of several FGF receptor antagonists such as the compound SU5402. Induction of endodermal differentiation can be assessed by measuring the phosphorylation state of intra-cellular Smad2 protein.

Differentiation towards endocrine pancreas could be biased by expression of key developmental genes in ES cells. For example, expression of pdx-1 under an appropriate promoter could be used to drive ES down the pancreatic lineage and help bias the cells to respond to the differentiation factors. The promoter could be a constitutive one such as CMV, SV40, EF1α, or beta-actin. Alternatively, the promoter could be an inducible one such as metallothionin, ecdysone, or tetracycline. The recombinant protein could also be tagged to a regulatory element such as the ligand-binding domain of the estrogen receptor or variants thereof. Such a fusion protein could be regulated by addition or withdrawal of estrogen analogs including tomaxifin. Recombinant DNA could be introduced in ES cells using a variety of methods including electroporation, lipofection, or transduction by viral agents such as adenovirus, lentivirus, herpes virus, or other pleiotropic viruses. In addition, inhibition of certain genes may help promote differentiation and/or help promote responsiveness of the ES to the differentiation factors. For example, inhibition of the smoothed/patched receptor, RBK-JK, or HES1 in hES-derived cells could help drive the ES cells toward the pancreatic lineage. Inhibition of the genes could be accomplished by antisense oligos, siRNA, deletion of endogenous alleles by homologous recombination or constitutive expression of an inhibitor or dominant negative gene.

Further Purification of Differentiated Cell Types

In certain embodiments, essentially purified preparations of one or more partially or terminally differentiated cell types of a particular tissue can be generated directly from ES cells or EBs. However, it may be necessary or preferable for certain applications of the differentiated cells to further expand or select particular differentiated cells. For example, such selection can be used to further purify a preparation of cells or can be used to, for example, take a preparation that includes multiple partially and/or terminally differentiated cell types and prepare a preparation that contains fewer, or even a single, partially and/or terminally differentiated cell types.

Cells differentiated from ES cells may need to be expanded and selected. One non-limiting approach is to express a drug resistant marker such as neo® under the control of a particular tissue specific promoter (e.g., the insulin promoter or pdx-1 promoter) in ES cells. As the cells undergo a differentiation protocol, the selection drug like G418 can be added to select for cells expressing the marker gene. Alternatively, one can tag a suicide gene such as diphtheria toxin to a particular promoter so as to eliminate cells that differentiate along an undesired lineage.

Reporter ES lines can also be used to monitor progression of differentiation. For example, a construct comprising GFP downstream of a particular promoter (e.g., the pdx-1 promoter or insulin promoter) could be introduced into ES cells. Cells that express the reporter can be readily detected. Other reporters genes that can be used include luciferase, alkaline phosphatase, lacZ, or CAT. Useful reporter lines could comprise multiple reporters to help identify cells that have differentiated along particular lineages. Cells expressing these reporters could be easily purified by FACS, antibody affinity capture, magnetic separation, or a combination thereof. The purified reporter-expressing cells can be used for genomic analysis by techniques such as microarray hybridization, SAGE, MPSS; or proteomic analysis to identify more markers that characterize the purified population. These methods can identify cells that have not differentiated along the desired lineages, as well as populations of cells that have differentiated along the desired lineages. In cultures containing too many cells that have not differentiated along the desired lineages, the desired cells may be isolated and subcultured to generate an essentially purified populations of one or more partially or terminally differentiated cell types of the desired tissue.

Reporter lines could be used in a high-throughput screening assay to rapidly screen for small molecules, growth factors, matrices, or different growth conditions that could favor differentiation along particular lineages. Screening platform could be 24, 48, 96, or even 384-wells. Detection method would depend on the type of reporter gene being expressed. For example, a luminescence plate reader could detect luciferase reporter and a fluorescent plate reader could detect GFP or even lacZ reporters. In addition, high content screening could be performed where automated microscopes would scan each well and measure several parameters including but not limited to the number of cells expressing the reporter gene, cell size, cell shape, and cell movement.

Pdx-1 positive cells or progenitors thereof could be further differentiated into endocrine cells based on methods detailed in U.S. Pat. No. 6,610,535 or as described in patent application PCT/US03/23852, the disclosures of each of which are hereby incorporated by reference in their entirety. Differentiating factors used in this protocol include but are not limited to FGF18, cardiotropin, PYY, forskolin, HGF, heparin, insulin, dexamethasone, follistatin, betacellulin, growth hormone, placental lactogen, EGF, KGF, IGF-I, IGF-II, VEGF, exendin-4, leptin, and nicotinamide, and notch antagonists.

ES cells could also be differentiated in vivo by injecting them into a SCID animal. It is well known that hES cells when injected into SCID mice could form teratomas that comprise tissues from all three germ layers. Injection into different tissues or organs may drive them down a particular lineage. The SCID mouse may also have a regenerating pancreas, where it has undergone partial pancreatectomy or treatment with streptozotocin. Alternatively, hES cells could be engrafted into different parts of an embryonic or fetal animal at various stage of development.

In one embodiment, ES cells are differentiated to produce essentially purified preparation of pancreatic cells. Such essentially purified preparations of pancreatic cells comprise one or more partially and/or terminally differentiated pancreatic cell type. In certain embodiment, the one or more partially and/or terminally differentiated pancreatic cell types include an insulin producing cell. ES-derived insulin-producing cells will preferably have the following characteristics: (i) express insulin mRNA as detected by RT-PCR, northern blot, or in situ hybridization; (ii) express insulin and C-peptide as detected by western blot or immunhistochemical staining; (iii) secrete insulin and C-peptide as detectable by ELISA or RIA; (iv) show glucose responsive insulin secretion; (v) rescue a diabetic animal (e.g., STZ-treated NOD/SCID mouse) when implanted in a suitable site of the animal.

During the course of differentiating ES cells into preparations including insulin-producing cells, it may be desirable to enrich the progenitors at different stages and further differentiate these progenitors. The progenitors may be purified by selecting cells expressing one or more pancreatic development genes, as outlined in detail above. The markers used for selection are preferably expressed on the cell surface so they are amenable to antibody affinity capture and sorting by either flow cytometry or magnetic cell separation. These markers may include dynein, gap junction membrane channel protein, integral membrane protein 2A, CXCR4, Sur-1, Glut-2, Kir6.2, microfibrillar-associated protein 2, procollagens, tachykinin2, Thy-1.2, tenascin C, vanin1, inward rectifier K+ channel J8, adaptor protein complex AP-1, microtubule-associated protein 1B, annexin A1, CD36, CD84, clusterin, catenin delta 2, endomucin, granulin, keratin Hb5, integrin α7, lysosomal membrane glycoprotein 2, KSPG, galectin-6, lipocortin I, mannose-binding lectin, lymphocyte antigen 64, synaptotagmin 4, thrombospondin, thrombomodulin, visinin-like 1, Fabp1, Fabp2, Slc25a5, Slc2a2, Slc7a8, Ep-cam, N-cadherin, E-cadherin, CK19, and CD31.

(iv) Exemplary Compositions

The methods of the present invention can be used to differentiate (partially or terminally) ES cells to one or more cell types of a tissue derived from the endodermal lineage. By way of example, the methods of the present invention can be used to differentiate ES cells to produce essentially purified preparations of one or more partially and/or terminally differentiated cells of the pancreas or liver. Such essentially purified preparations of one or more partially and/or terminally differentiated cells can be formulated in a pharmaceutically acceptable carrier and administrated to patients suffering from a condition characterized by a decrease in functional performance of a particular endodermally derived organ.

In one embodiment, ES cells are differentiated to produce essentially purified preparation of pancreatic cells. Such essentially purified preparations of pancreatic cells comprise one or more partially and/or terminally differentiated pancreatic cell type. In certain embodiments, the one or more partially and/or terminally differentiated pancreatic cell types include an insulin producing cell. ES-derived insulin-producing cells will preferably have the following characteristics: (i) express insulin. mRNA as detected by RT-PCR, northern blot, or in situ hybridization; (ii) express insulin and C-peptide as detected by western blot or immunhistochemical staining; (iii) secrete insulin and C-peptide as detectable by ELISA or RIA; (iv) show glucose responsive insulin secretion; (v) rescue a diabetic animal (e.g., STZ-treated NOD/SCID mouse) when implanted in a suitable site of the animal.

(v) Application to Other Stem Cell Populations

The present invention provides methods for directing the differentiation of embryonic stem cells to endodermal cell types. However, the methods provided in the present application are not limited to modulating the differentiation of embryonic stem cells. Embryonic stem cell have a limitless differentiation potential. However, this limitless potential has proved challenging to researchers trying to direct the differentiation of these cells along particular lineages, in a controlled manner, and as a commercially and therapeutically useful percentage of a cell culture. In contrast, many adult stem cell populations have actually proven more amenable to directed differentiation. Accordingly, given that the methods provided herein effectively promote directed differentiation of embryonic stem cells to endodermal cell types, the invention contemplates that these methods will similarly be able to direct the differentiation of other adult stem cells, for example, stem cells derived from a fetal or adult animal tissue. Exemplary adult stem cells include, but are not limited to, hematopoietic stem cells, neuronal stem cells, neural crest stem cells, mesenchymal stem cells, myocardial stem cells, pancreatic stem cells, hepatic stem cells, and endothelial stem cells. Further exemplary adult stem cells can be derived from virtually any organ or tissue including, but not limited to, tongue, skin, esophagus, brain, spinal cord, endothelium, hair follicle, stomach, small intestine, large intestine, ovary, testes, blood, bone, bone marrow, umbilical cord, lung, gall bladder, and the like. In one embodiment, the adult stem cell is a stem cell population which can differentiate along an endodermal lineage using either the methods of the present invention or other methodologies known in the art.

(vi) Methods of Treatment

The present invention provides a variety of methods for promoting the directed differentiation of embryonic stem cells to particular differentiated cell types. In certain embodiments, the invention provides methods for promoting the directed differentiation of embryonic stem cells along a pancreatic lineage. Exemplary methods result in production of cells and cell clusters expressing pdx-1+, insulin, and/or C-peptide. Furthermore exemplary methods result in production of cells and cell clusters that release C-peptide. The present invention further provides substantially purified cultures of cells and cell clusters (e.g., partially or terminally differentiated cells) differentiated from embryonic stem cells. By substantially purified is meant that a culture of differentiated cells or cell clusters contains less than 20%, preferably less than 15%, 10%, 7%, 5%, 4%, 3%, 2%, 1%, or less than 1% of cells that are either undifferentiated or differentiated to a cell type of a different (e.g., a non-pancreatic lineage).

Substantially purified cells and cell clusters differentiated along a pancreatic lineage by the methods of the present invention can be used therapeutically for treatment of various disorders associated with injury, disease, or other decrease in the functional performance of the pancreas. Substantially purified cultures of cells for use in the therapeutic methods of the invention include essentially homogenous cultures of cells (e.g., essentially all of the cells are of a particular partially or terminally differentiated cell type) or heterogenous cultures of cells. When heterogenous cultures of cells are used essentially all of the cells are derived from a particular lineage and are related to a particular tissue type (e.g., the culture comprises various partially differentiated and/or terminally differentiated pancreatic cell types such as a mixture of pdx-1+ and insulin+ cell types).

To illustrate, the methods of the present invention can be used to generate partially or terminally differentiated pancreatic cell types. Such pancreatic cell types can be used in the treatment or prophylaxis of pancreatic disorders, both exocrine and endocrine, as well as pancreatic injuries. In one embodiment, the methods of the invention can be used to produce pancreatic beta-like cells or cell clusters useful for the treatment of diabetes or other conditions of impaired glucose regulation. By pancreatic beta-like cells or cell clusters is meant that the cells or cell clusters express pancreatic genes including, but not limited to, pdx-1, insulin, and/or C-peptide. In certain embodiments, the pancreatic beta-like cells or cell clusters secrete C-peptide and are glucose responsive.

In addition to particular disease states, the methods of the present invention can be used to generate partially or terminally differentiated cell types for the treatment of an injury to the particular organ or tissue. Such injuries include, but are not limited to, blunt trauma, surgical resection, or tissue damage caused by cancer or other proliferative disorder.

In any of the foregoing, the invention also contemplates that preparations of partially and/or terminally differentiated cell types of an endodermally derived tissue may be useful for other purposes in addition to therapeutic purposes. Such cells may be useful in screens to identify non-cell agents that promote proliferation, differentiation, survival, or migration of the differentiated cells.

The present invention provides partially and/or terminally differentiated endodermal cell types that can be used to treat or prophylactically treat injury, disease, or other decrease in functional performance of an endodermally derived tissue. Such cells can be administered directly to the affected tissue (e.g., via transplantation or injection directly or adjacent to the affected tissue). Such cells can also be administered systemically (e.g., via intravenous injection) and allowed to home to the site of disease or damage (e.g., to home to the affected tissue following systemic administration).

Additionally, the invention contemplates that preparations of partially and/or terminally differentiated cells can be administered alone or can be administered in combination with other therapies. By way of example, the cells can be administered concurrently to or concomitantly with one or more agents that promotes one or more of proliferation, differentiation, migration, or survival. Without wishing to be bound by theory, such agents may, for example, help transplanted cells home to the site of damage. Furthermore, such agents may help promote the survival of both the endogenous tissue and the transplanted cells. Such agents can be used to treat conditions associated, in whole or in part, by loss of, injury to, or decrease in functional performance of endodermal cell types.

The following are illustrative of disease states that can be treated using preparations of cells differentiated along a pancreatic lineage from ES cells. Such diseases can be treated using (i) preparations of differentiated cells alone, (ii) preparations of differentiated cells in combination with one or more non-cell based compounds or agents, or (iii) preparation of differentiated cells in combination with one or more treatment regimens appropriate for the particular disease or injury being treated.

Exemplary diseases

Pancreatic Diseases

1. Diabetes Mellitus

Diabetes mellitus is the name given to a group of conditions affecting about 17 million people in the United States. The conditions are linked by their inability to create and/or utilize insulin. Insulin is a hormone produced by the beta cells in the pancreas. It regulates the transportation of glucose into most of the body's cells, and works with glucagon, another pancreatic hormone, to maintain blood glucose levels within a narrow range. Most tissues in the body rely on glucose for energy production.

Diabetes disrupts the normal balance between insulin and glucose. Usually after a meal, carbohydrates are broken down into glucose and other simple sugars. This causes blood glucose levels to rise and stimulates the pancreas to release insulin into the bloodstream. Insulin allows glucose into the cells and directs excess glucose into storage, either as glycogen in the liver or as triglycerides in adipose (fat) cells. If there is insufficient or ineffective insulin, glucose levels remain high in the bloodstream. This can cause both acute and chronic problems depending on the severity of the insulin deficiency. Acutely, it can upset the body's electrolyte balance, cause dehydration as glucose is flushed out of the body with excess urination and, if unchecked, eventually lead to renal failure, loss of consciousness, and death. Over time, chronically high glucose levels can damage blood vessels, nerves, and organs throughout the body. This can lead to other serious conditions including hypertension, cardiovascular disease, circulatory problems, and neuropathy.

2. Pancreatitis

Pancreatitis can be an acute or chronic inflammation of the pancreas. Acute attacks often are characterized by severe abdominal pain that radiates from the upper stomach through to the back and can cause effects ranging from mild pancreas swelling to life-threatening organ failure. Chronic pancreatitis is a progressive condition that may involve a series of acute attacks, causing intermittent or constant pain as it permanently damages the pancreas.

Normally, the pancreatic digestive enzymes are created and carried into the duodenum (first part of the small intestine) in an inactive form. It is thought that during pancreatitis attacks, these enzymes are prevented or inhibited from reaching the duodenum, become activated while still in the pancreas, and begin to autodigest and destroy the pancreas. While the exact mechanisms of pancreatitis are not well understood, it is more frequent in men than in women and is known to be linked to and aggravated by alcoholism and gall bladder disease (gallstones that block the bile duct where it runs through the head of the pancreas and meets the pancreatic duct, just as it joins the duodenum). These two conditions are responsible for about 80% of acute pancreatitis attacks and figure prominently in chronic pancreatitis. Approximately 10% of cases of acute pancreatitis are due to idiopathic (unknown) causes. The remaining 10% of cases are due to any of the following: drugs such as valproic acid and estrogen; viral infections such as mumps, Epstein-Barr, and hepatitis A or B; hyper-triglyceridemia, hyperparathyroidism, or hypercalcemia; cystic fibrosis or Reye's syndrome; pancreatic cancer; surgery in the pancreas area (such as bile duct surgery); or trauma.

Acute Pancreatitis

About 75% of acute pancreatitis attacks are considered mild, although they may cause the patient severe abdominal pain, nausea, vomiting, weakness, and jaundice. These attacks cause local inflammation, swelling, and hemorrhage that usually resolves itself with appropriate treatment and does little or no permanent damage. About 25% of the time, complications develop, such as tissue necrosis, infection, hypotension (low blood pressure), difficulty breathing, shock, and kidney or liver failure.

Chronic Pancreatitis

Patients with chronic pancreatitis may have recurring attacks with symptoms similar to those of acute pancreatitis. The attacks increase in frequency as the condition progresses. Over time, the pancreas tissue becomes increasingly scarred and the cells that produce digestive enzymes are destroyed, causing pancreatic insufficiency (inability to produce enzymes and digest fats and proteins), weight loss, malnutrition, ascities, pancreatic pseudocysts (fluid pools and destroyed tissue that can become infected), and fatty stools. As the cells that produce insulin and glucagons are destroyed, the patient may become permanently diabetic.

3. Pancreatic Insufficiency

Pancreatic insufficiency is the inability of the pancreas to produce and/or transport enough digestive enzymes to break down food in the intestine and allow its absorption. It typically occurs as a result of chronic pancreatic damage caused by any of a number of conditions. It is most frequently associated with cystic fibrosis in children and with chronic pancreatitis in adults; it is less frequently but sometimes associated with pancreatic cancer.

Pancreatic insufficiency usually presents with symptoms of malabsorption, malnutrition, vitamin deficiencies, and weight loss (or inability to gain weight in children) and is often associated with steatorrhea (loose, fatty, foul-smelling stools). Diabetes also may be present in adults with pancreatic insufficiency.

In the treatment of any of the above mentioned conditions, the dosage (e.g., what constitutes a therapeutically effective amount of differentiated cells) is expected to vary from patient to patient depending on a variety of factors. The selected dosage level will depend upon a variety of factors including the specific condition to be treated, other drugs, compounds and/or materials used in combination with the particular cell-based therapy, the severity of the patient's illness, the age, sex, weight, general health and prior medical history of the patient, and like factors well known in the medical arts.

A physician or veterinarian having ordinary skill in the art can readily determine and prescribe the effective amount of the pharmaceutical composition required. For example, the physician or veterinarian could start doses of the cells of the invention employed in the pharmaceutical composition at levels lower than that required in order to achieve the desired therapeutic effect and gradually increase the dosage until the desired effect is achieved.

In general, a suitable dose of cells of the invention will be that amount of the cells which is the lowest dose effective to produce a therapeutic effect. Such an effective dose will generally depend upon factors including the patient's age, sex, and the severity of their injury or disease.

In the case of the present invention, the pharmaceutical composition comprises cells differentiated by the methods of the present invention and one or more pharmaceutically acceptable carriers or excipients. As outlined above, the pharmaceutical composition may be administered in any of a number of ways including, but not limited to, systemically, intraperitonially, directly transplanted, and furthermore may be administered in association with hollow fibers, tubular membranes, shunts, or other biocompatible devices or scaffolds.

The term “treatment” is intended to encompass also prophylaxis, therapy and cure, and the patient receiving this treatment is any animal in need, including primates, in particular humans, and other mammals such as equines, cattle, swine and sheep; as well as poultry and pets in general.

The present invention provides methods for directing the differentiation of embryonic stem cells to produce cultures of endodermally derived cells. Such cultures can optionally be further purified to enrich for particular cell types, thereby providing an essentially purified preparation of endodermally derived cell types.

Essentially purified preparations of partially and/or terminally differentiated endodermally derived cells can be used therapeutically to treat or prophylactically treat injuries or disease of the particular organ or tissue. For example, essentially purified preparations of pancreatic cells can be formulated in a pharmaceutically acceptable carrier and delivered to patients suffering from a condition characterized by loss in functional performance of the pancreas (e.g., diabetes).

When preparations of cells are delivered to patients, the invention contemplates that the therapeutic treatment additionally comprises administering other therapeutic agents. By way of example, agents that inhibit cell death, promote cell survival, or promote cell migration can be administered concurrently or concomitantly with the preparation of differentiated cells. Furthermore, the invention contemplates therapeutic methods comprising administration of the subject cells concurrently or concomitantly with other therapeutic regimens appropriate to treat the particular condition being treated (e.g., cells+insulin for the treatment of diabetes).

When the therapeutic method involves administration of cells and one or more additional agents or treatment modalities, the invention contemplates that the cells and agents can be administrated via the same method of administration or via different methods of administration. By way of non-limiting example, the invention contemplates that in certain embodiments, preparations of terminally and/or partially differentiated pancreatic cells will be surgically or laproscopically transplanted directly to the abdominal cavity or directly to endogenous pancreatic tissue. If one or more additional agents are also part of the particular treatment protocol, such agents may be similarly delivered, or may be delivered in another manner (e.g., injected intravenously, transdermally, orally, subcutaneous, etc.).

The pharmaceutical compositions/preparations of the present invention (e.g., pharmaceutical compositions of cells and pharmaceutical compositions of non-cell agents/compounds) are formulated according to conventional pharmaceutical compounding techniques. See, for example, Remington's Pharmaceutical Sciences, 18th Ed. (1990, Mack Publishing Co., Easton, Pa.). Pharmaceutical formulations of the invention can contain the active polypeptide and/or agent, or a pharmaceutically acceptable salt thereof. These compositions can include, in addition to an active polypeptide and/or agent, a pharmaceutically acceptable excipient, carrier, buffer, stabilizer or other material well known in the art. Such materials should be non-toxic and should not interfere with the efficacy of the active agent. Preferable pharmaceutical compositions are non-pyrogenic. The carrier may take a wide variety of forms depending on the route of administration, e.g., intravenous, intravascular, oral, intrathecal, epineural or parenteral, transdermal, etc. Furthermore, the carrier may take a wide variety of forms depending on whether the pharmaceutical composition is administered systemically or administered locally, as for example, via surgical transplantation, laproscopic transplantation, or via a biocompatible device (e.g., catheter, stent, wire, or other intraluminal device).

Illustrative examples of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

In one embodiment, the pharmaceutical composition is formulated for sustained-release. An exemplary sustained-release composition has a semi permeable matrix of a solid biocompatible polymer to which the composition is attached or in which the composition is encapsulated. Examples of suitable polymers include a polyester, a hydrogel, a polylactide, a copolymer of L-glutamic acid and ethyl-L-glutamase, non-degradable ethylene-vinyl acetate, a degradable lactic acid-glycolic acid copolymer, and poly-D+-hydroxybutyric acid.

Polymer matrices can be produced in any desired form, such as a film, or microcapsules.

Other sustained-release compositions include liposomally entrapped modified compositions. Liposomes suitable for this purpose can be composed of various types of lipids, phospholipids, and/or surfactants. These components are typically arranged in a bilayer formation, similar to the lipid arrangement of biological membranes.

Pharmaceutical compositions according to the invention include implants, i.e., compositions or device that are delivered directly to a site within the body and are, preferably, maintained at that site to provide localized delivery.

As outlined above, biocompatible devices for use in the various methods of delivery contemplated herein can be composed of any of a number of materials. The biocompatible devices include wires, stents, catheters, balloon catheters, and other intraluminal devices. Such devices can be of varying sizes and shapes depending on the intended vessel, duration of implantation, particular condition to be treated, and overall health of the patient. A skilled physician or surgeon can readily select from among available devices based on the particular application.

By way of further illustration, exemplary biocompatible, intraluminal devices are currently produced by several companies including Cordis, Boston Scientific, Guidant, and Medtronic (Detailed description of currently available catheters, stents, wires, etc., are available at the websites of Cordis Corporation (cordis.com); Medtronic, Inc. (medtronic.com); and Boston Scientific Corporation (bostonscientific.com)).

The invention also provides articles of manufacture including pharmaceutical compositions of the invention and related kits. The invention encompasses any type of article including a pharmaceutical composition of the invention, but the article of manufacture is typically a container, preferably bearing a label identifying the composition contained therein.

The container can be formed from any material that does not react with the contained composition and can have any shape or other feature that facilitates use of the composition for the intended application. A container for a pharmaceutical composition of the invention intended for parental administration generally has a sterile access port, such as, for example, an intravenous solution bag or a vial having a stopper pierceable by an appropriate gauge injection needle.

Cell-based and/or non-cell-based compositions for use in the therapeutic methods of the present invention may be conveniently formulated for administration with a biologically acceptable medium, such as water, buffered saline, polyol (for example, glycerol, propylene glycol, liquid polyethylene glycol and the like) or suitable mixtures thereof. For therapeutic methods comprising administration of both cell-based and non-cell based compositions, the invention contemplates that such compositions may be formulated in the same or different carriers. The appropriate formulation and medium can be chosen based on the mode of administration.

Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Compositions of the present invention may be given orally, parenterally, or topically. They are of course given by forms suitable for each administration route. For example, they are administered in tablets or capsule form, by injection, inhalation, ointment, controlled release device or patch, or infusion.

The effective amount or dosage level will depend upon a variety of factors including the activity of the particular compositions employed, the route of administration, the time of administration, the rate of excretion of the particular compositions being employed, the duration of the treatment, other drugs, compounds and/or materials used in combination with the particular compositions employed, the age, sex, weight, condition, general health and prior medical history of the animal, and like factors well known in the medical arts.

The compositions (e.g., cell-based compositions alone or in combination with one or more non-cell based compositions) can be administered as such or in admixtures with pharmaceutically acceptable and/or sterile carriers and can be administered concomitantly or concurrently with other compounds.

Thus, another aspect of the present invention provides pharmaceutically acceptable compositions comprising an effective amount of cell or non-cell based compositions, formulated together with one or more pharmaceutically acceptable carriers (additives) and/or diluents. As described below, the pharmaceutical compositions of the present invention may be specially formulated for administration in solid or liquid form, including those adapted for the following: (1) delivery via a catheter, port or other biocompatible, intraluminal device; (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension. Furthermore, the cells or cell clusters may be surgically or laproscopically implanted either near the pancreas or in the abdominal cavity, or at a distant and more accessible site. In certain embodiments the subject compositions may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Compositions for administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an composition, it is desirable to slow the absorption of the agent from subcutaneous or intramuscular injection. This may be accomplished by the use of a liquid suspension of crystalline or amorphous material having poor water solubility. The rate of absorption of the composition then depends upon its rate of dissolution which, in turn, may depend upon crystal size and crystalline form. Alternatively, delayed absorption of a parenterally administered composition form is accomplished by dissolving or suspending the agent in an oil vehicle.

For any of the foregoing, the invention contemplates administration to neonatal, adolescent, and adult patients, and one of skill in the art can readily adapt the methods of administration and dosage described herein based on the age, health, size, and particular disease status of the patient. Furthermore, the invention contemplates administration in utero to treat conditions in an affected fetus.

(vii) Immune Tolerance

One issue that may arise with any therapeutic intervention involving the delivery of xenogenic cells or tissue is that of rejection. For example, despite the efforts made to minimize antigen mismatch prior to whole organ transplantation, graft rejection remains a serious limiting factor in the long-term efficacy of transplanted organs, as well as of transplantation patients.

Although some reports suggest that embryonic stem cells, even xenogenic stem cells, will not provoke an immune response, it is unclear whether this will in practice be true. Furthermore, even if embryonic stem cells themselves do not provoke an immune response, progeny of ES cells differentiated in vitro may provoke an immune response. Accordingly, the invention contemplates therapeutic methods comprising administration of pharmaceutical preparations concurrently or concomitantly with immuno-suppressants and/or other anti-rejection drugs. As outlined in detail above, when the therapeutic methods involve administration of both cell-based and non-cell based compositions, the invention contemplates administration via the same or via a different mode of administration, and similarly contemplates that the compositions are each formulated appropriately in light of their properties and the desired route of administration.

In addition to immuno-suppression via traditional anti-rejection drugs, the invention contemplates additional methods of preventing host rejection of the differentiated cells. Such methods are based on inducing tolerance in the patient, and can be used alone or in combination with other immunosuppresants or anti-rejection drugs.

In one embodiment, tolerance is induced by first introducing into the patient dendritic cells differentiated from the same line of ES cells that will be used to differentiate the particular endodermal cells. ES cells can be differentiated into dendritic cells by first driving them down the hematopoietic lineage via addition of one or more factors including; but not limited to, IL-1, IL-3, IL-6, GM-CSF, G-CSF, SCF, or erythropoietin. Alternatively, the ES cells can be co-cultured with cell lines such as OP-9 stromal cells or yolk-sac endodermal cells.

Following differentiation of ES cells to dendritic cells, essentially purified populations of dendritic cells can be prepared and delivered to the patient. Such dendritic cells are delivered to the patient prior to administration of the therapeutic cells (e.g., the pancreatic cells or the hepatic cells). The dendritic cells are optionally delivered along with traditional immunosuppressive therapies. When the therapeutic cells are later delivered, they may optionally be delivered with the same or with a lower dose of immunosuppresants.

When the methods of the present invention are used to direct the differentiation of non-embryonic stem cells, the invention contemplates that these partially or terminally differentiated adult stem cells can be used therapeutically in all of the ways described for embryonic stem cells. When adult stem cells are used, potential graft rejection can be eliminated by using cells derived from the patient to be treated. Alternatively, the above contemplated immunosuppressive and tolerance approaches are also contemplated.

### EXEMPLIFICATION

The invention now being generally described, it will be more readily understood by reference to the following examples which are included merely for purposes of illustration of certain aspects and embodiments of the present invention, and are not intended to limit the invention in any respect.

### Example 1

### Human Embryonic Stem Cells Spontaneously Differentiate to Ectodermal, Mesodermal, and Endodermal Cell Types

FIG. 1 confirms previous experiments demonstrating that human embryonic stem (ES) cells spontaneously differentiate along all three lineages when cultured as embryoid bodies (EBs). Human embryonic stem cell lines 1 or 2 (hES1 and hES2) were used to generate embryoid bodies. Briefly, ES cells were removed from the MEF feeder layer by either manual cutting (M) or collagenase digestion (C). The removed ES cells were then placed in appropriate media. After 0, 5, or 9 days post-EB formation, RNA was extracted from the EBs and analyzed for expression of the indicated markers by real-time RT-PCR. Relative expression shown is normalized to that of β-actin and expression for day 0 was set equal to 1. An asterisk (*) indicates arbitrary values due to no expression at day 0. Data is shown for two hES lines—hES1 and hES2, with hES2 shown in parenthesis. There was no significant change in expression for sox17, nkx6.1, and brachyury.

### Example 2

### Methods for Generating Embryoid Bodies

One method for directing the differentiation of stem cells is to generate embryoid bodies. These embryoid bodies can be grown under a number of conditions including, but not limited to, in floating suspension culture, in MATRIGEL™ or other matrix, or on a filter. However, the first step is the actual formation of an embryoid body from a culture of embryonic stem cells. We used any of the following methods for generating embryoid bodies from cultures of embryonic stem cells. These methods can be used to generate embryoid bodies from human embryonic stem cells grown on MEF feeder layers, embryonic stem cells grown on other feeder layers, and embryonic stem cells grown under feeder free conditions.

Materials: Human embryonic stem cells (e.g., lines hES 1-6 or DM lines); culture medium; PBS; collagenase IV stock solution (5 mg/ml)—preferably for use with hES 1-6; trypsin/EDTA stock solution (0.25%)—preferably for use with DM lines; ultra-low-6-well plates.

(a) Collagenase EB Protocol

The following protocol can be used to generate embryoid bodies. This protocol was specifically used to generate embryoid bodies from cell lines hES 1-6. However, the protocol can be used more generally in other ES cell or cell lines.

P100 tissue culture plates containing hES cells grown under standard conditions were used as starting material. The medium was aspirated, and the cells were washed 2 times with PBS. After the wash, 3 ml of 1 mg/ml collagenase IV was added to each plate, and the plates were incubated for 8 minutes in a 37° C. tissue culture incubator. Following incubation, the collagenase was aspirated from the cells, and the cells were washed with 10 ml of PBS. The PBS was gently aspirated, and care was taken to avoid disturbing the colonies of embryonic stem cells.

About 8 ml of EB culture medium was gently added to the plate, and the plate was mechanically and gently streaked using a 5-ml plastic pipette or a cell scraper. The materials were pipetted up and down to dislodge the cells pieces—care was taken not to over-pipette and damage the cells. The embryonic stem cell clusters were transferred to ultra-low 6-well plates to promote embryoid body formation. Embryoid bodies were cultured for several days, and EB culture medium was changed every 2-3 days.

When experiments called for analysis of gene or protein expression in EBs, the EBs were handled as follows: EBs were collected in a tube, and were allowed to either settle to the bottom of the tube, or were spun briefly to facilitate precipitation of the EBs to the bottom of the tube. At this point, EBs can be processed for immunohistochemistry studies or for RNA extraction, using standard techniques.

(b) Collagenase EB protocol for Manually-Passaged hES1-6

The following protocol can be used to generate embryoid bodies. This protocol was specifically used to generate embryoid bodies from cell lines hES 1-6. However, the protocol can be used more generally in other ES cell or cell lines.

Organ cultures dishes containing hES cells grown under standard conditions on MEF feeder layers were used as starting material. The medium was aspirated, and the cells were washed 2 times with PBS. Then 0.5 ml of 1 mg/ml collagenase IV was added to each dish, and the plates were incubated for 5 minutes in a 37° C. tissue culture incubator. Following incubation, the collagenase was aspirated from the cells, and the cells were washed with 1 ml of PBS. The PBS was gently aspirated, and care was taken to avoid disturbing the colonies of embryonic stem cells.

About 1 ml of EB culture medium was gently added to the plate. ES cell colonies were dissociated gently using a pipet tip. Care was taken to avoid detaching MEFs from the dish. ES cell pieces were transferred to a suspension plate containing EB culture medium to promote embryoid body formation. Embryoid bodies were cultured for several days, and EB culture medium was changed every 2-3 days.

When experiments called for analysis of gene or protein expression in EBs, the EBs were handled as follows: EBs were collected in a tube, and were allowed to either settle to the bottom of the tube, or were spun briefly to facilitate precipitation of the EBs to the bottom of the tube. At this point, EBs can be processed for immunohistochemistry studies or for RNA extraction, using standard techniques.

(c) Collagenase EB Protocol for Trypsin-Passaged Harvard HUES-1 Cells

The following protocol can be used to generate embryoid bodies. This protocol was specifically used to generate embryoid bodies from the Harvard cell line HUES-1. However, the protocol can be used more generally in other ES cell or cell lines.

P100 tissue culture plates containing hES cells grown under standard conditions on MEF feeder layers were used as starting material. The process begins with a stage whereby the ES cells were waned from the feeder layer. The cells were trypsinized with 0.05% trypsin and plated on collagen IV-coated plates in the splitting ratio of 1:3. The cells were cultured for 34 until subconfluent.

Following this waning phase, the medium was aspirated, and the cells were washed 2 times with PBS. About 3 ml of 1 mg/ml collagenase IV was added to each dish, and the plates were incubated for 4 minutes in a 37° C. tissue culture incubator. Following incubation, the collagenase was aspirated from the cells, and the cells were washed with 10 ml of PBS. The PBS was gently aspirated, and care was taken to avoid disturbing the colonies of embryonic stem cells.

About 8 ml of EB culture medium was gently added to the plate, and the plate was mechanically scraped using a 5-ml plastic pipette and cell scraper. The materials were pipetted up and down to dislodge the cells pieces—care was taken not to over-pipette and damage the cells. The embryonic stem cell clusters were transferred to suspension plates to promote embryoid body formation. Embryoid bodies were cultured for several days, and EB culture medium was changed every 2-3 days.

When experiments called for analysis of gene or protein expression in EBs, the EBs were handled as follows: EBs were collected in a tube, and were allowed to either settle to the bottom of the tube, or were spun briefly to facilitate precipitation of the EBs to the bottom of the tube. At this point, EBs can be processed for immunohistochemistry studies or for RNA extraction, using standard techniques.

### Example 3

### Method for Directing the Differentiation of a Stem Cell to a Particular Differentiated Cell Type

The following is indicative of protocols that can be used to direct the differentiation of stem cells to a particular differentiated cell type. The particular protocol outlined here promoted differentiation of embryonic stem cells along the pancreatic lineage, as assayed by expression of the marker pdx-1.

Materials: Human embryonic stem cells; medium (RPMI/20% serum replacement (20SR)/pen-strep; PBS; collagenase IV (preferably for use with hES 1-6 lines); trypsin/EDTA (preferably for DM lines); ultra-low attachment-6-well plates (Corning/costar) growth factor reduced MATRIGEL™; early factors (EF); late factors (LP).

Exemplary Time table:

| | |
|---|---|
| D-1 | Liquefy MATRIGEL ™ on ice and keep |
| | ice box in the cold room overnight. |
| D0-D10 | Early factor stage |
| D0 | Make MATRIGEL ™ EB. |
| D3 | Top up with RPMI/20SR (0.5 ml) + EF |
| | (quantity for 2 ml medium). |
| D6 | Top up with RPMI/20SR (0.5 ml) + EF |
| | (quantity for 2 ml medium). |
| D10-D20 | Late factor stage |
| D10 | Wash away early factors. Change to LF. |
| D13 | Top up with RPMI/20SR (0.5 ml) + LF |
| | (quantity for 2 ml medium). |
| D16 | Top up with RPMI/20SR (0.5 ml) + LF |
| | (quantity for 2 ml medium). |
| D20 | Harvest MATRIGEL ™ EB for |
| | RNA-RT-PCR, immuno-staining or in situ hybridization. |

D0 in the time table provided above indicates the point at which culture of cells as embryoid bodies begin. Alternatively, for embodiments in which the cells are differentiated without embryoid body formation, D0 indicates the point at which the embryonic stem cells are plated directly onto MATRIGEL™ or other tissue culture plates. Prior to D0, cultures of proliferating ES cells must be handled according to one of the protocol outlined above to generate a starting culture of EBs.

Exemplary Experimental Procedure

Part 1: Collagenase Treatment of hES1-6

P100 tissue culture plates containing hES cells grown under standard conditions were used as starting material. The medium was aspirated, and the cells were washed 2 times with PBS. About 3 ml of 1 mg/ml collagenase IV was added to each plate, and the plates were incubated for 8 minutes in a 37° C. tissue culture incubator. Following incubation, the collagenase was aspirated from the cells, and the cells were washed with 10 ml of PBS. The PBS was gently aspirated, and care was taken to avoid disturbing the colonies of embryonic stem cells.

About 8 ml of EB culture medium (RPMI/20SR) was gently added to the plate, and the plate was mechanically scraped using a 5-ml plastic pipette and cell scraper. The materials were pipetted up and down to dislodge the cells pieces—care was taken not to over-pipette and damage the cells. The pellets were transferred to a 15-ml tube, and the pellets were spun down at 2500 rpm for 4 minutes. The medium was aspirated.

Part 2: Make MATRIGEL™ EB

Early Factor Stage

Day 0

1:6 MATRIGEL™ medium (e.g., 1 ml liquefied MATRIGEL™+5 ml RPMI/20SR) was prepared using pre-chilled pipettes. Total volume is according to 2 ml for each well. The hES cell pellets were resuspended in the MATRIGEL™ medium, and 2 ml of hES pellet:medium suspension was added/well of Ultra-low plate.

For wells in which growth factors were to be added, factor cocktail (100 ng each) can either be added in the MATRIGEL™ medium before pellet resuspension procedure or immediately after suspension is plated in the Ultra-low plate. Plates were incubated in 37° C. tissue culture incubator, and the MATRIGEL™ medium gels after several hours. After overnight incubation, the hES pellets formed embedded embryoid bodies.

Day 3 and 6

EB cultures were supplemented with 0.5 ml additional RPMI/20SR medium+early factors (100 ng of each factor).

Late Factor Stage

D10

The medium was removed. Care was taken to prevent dislodgement of the EBs and the MATRIGEL™. Fresh medium was added to the cells which were allowed to equilibrate for approximately 1 hour. This was followed by the addition of the LF cocktail and RPMI/20SR.

D13 and 16

EB cultures were supplemented with 0.5 ml additional RPMI/20SR medium+late factors.

D20

The MATRIGEL™ EBs were collected into a 15 ml FALCON tube. 12 ml chilled PBS was added, and the EBs were placed on ice for 10 min. The EBs were spun for 4 minutes at 2500 rpm (round per minute). The supernatant was carefully removed, and the EBs were transferred to an EPPENDORF tube. The EBs were then analyzed using immunocytochemistry or RT-PCR.

### Example 4

### Schematic Representation of Multi-Step Method for Differentiating Stem Cells Along Particular Endodermal Lineages

FIG. 2 provides a schematic representation of a multi-step method for directing the differentiation of stem cells along particular endodermal lineages. For the particular embodiment illustrated in FIG. 2, the starting material is embryonic stem cells, and the particular endodermal lineage is pancreatic—specifically beta islet cells. Embryonic stem cells can be cultured in any of a number of formats, for example, as embryoid bodies in suspension culture, as embryoid bodies embedded in MATRIGEL™ or other matrix material, as embryoid bodies on a filter, as embryonic stem cells directly plated on MATRIGEL™ or other matrix, or as embryonic stem cells directly plated on tissue culture plates. Regardless of the particular format, embryonic cells cultured in any of the foregoing ways are cultured for 1-10 days (or even 1-15 days) in medium containing early factors. This period of culture directs the cells down a particular endodermal pathway. For pancreatic cell types, culture in early factors results in partial differentiation, as assessed by expression of the early marker Pdx-1. Exemplary early factors that help to induce the expression of pdx-1 include, but are not limited to, activin A, BMP2, BMP4, and nodal. Such factors can be added individually or in combination. Combinations include combinations of two, three, four, or more than four factors.

After this first stage of differentiation, cells are cultured for 1-10 days (or even 1-15 days) in medium supplemented with late factors. This period of culture further promotes differentiation of cells along the particular pathway toward terminal differentiation. This may include promotion of further expression of pdx-1, promotion of expression of terminal markers of differentiation, both promotion of further expression of pdx-1 and further expression of insulin, or decrease expression of pdx-1 accompanied by an increased expression of markers of terminal differentiation.

For pancreatic cell types, and specifically for beta islet cells, culture in late factors promotes further differentiation. Promotion of further differentiation can be assessed by assaying for a further increase in expression of pdx-1. Additionally or alternatively, further differentiation can be assayed by expression of late markers including insulin. Note that pdx-1 expression is maintained, although perhaps at a lower level, upon terminal differentiation of the cells.

Exemplary late factors that help induce expression of markers of terminal beta islet differentiation include, but are not limited to, HGF, exendin4, betacellulin, and nicotinamide. Such factors can be added individually or in combination. Combinations include combinations of two, three, four, or more than four factors.

At this point, cells may optionally be further cultured to enhance terminal differentiation and functional performance.

### Example 5

### Multi-Step Method for Differentiating Stem Cells Along Particular Endodermal Lineages

Human embryonic stem cell 3 (hES3) were subjected to a multi-step differentiation protocol, as outlined in FIG. 2. The embryonic stem cells were cultured as embryoid bodies suspended in 3D in MATRIGEL™. The cells were cultured for 10 days in medium containing the early factors and then for 10 days in medium containing the late factors. Following culture, cells were assayed for expression of pdx-1.

FIG. 3 summarizes the results of these experiments. For each bar depicted in FIG. 3, the embryoid bodies were cultured, except as indicated, with the following early and late factors: early factors were activin A, BMP2, BMP4, and nodal; late factors were HGF, exendin4, betacellulin, and nicotinamide. The particular factor omitted is indicated under each bar.

As shown in FIG. 3, culture of the embryoid bodies in the presence of all of the early factors and all of the late factors resulted in an approximately four fold increase in expression of pdx-1 in comparison to culture in the absence of these growth factors. However, the use of all of these factors was not essential to induce robust expression of pdx-1. For example, the inclusion of BMP2, nodal, betacellulin, and nicotinamide appears optional.

Further, the role of MATRIGEL™ in differentiation was investigated. Some hES3-derived embryoid bodies were cultured free-floating for 20 days in RPMI media alone (−) or in MATRIGEL™ 1:6/RPMI for the times indicated in the presence of the 2EF and 3LF growth factors. On day 20, cells were analyzed for the expression of Pdx-1. As shown in FIG. 30, Pdx-1 expression was prominently enhanced in cells that were cultured in MATRIGEL™ between days 0 and 10, but not so if the cells were cultured in MATRIGEL™ between days 10 and 20. These data show that the requirement for MATRIGEL™ is restricted to about days 0 and 10 (when the EBs are in contact with the EFs). Continued presence of MATRIGEL™ from days 10-20 is only marginally beneficial, if anything at all. The lack of MATRIGEL™ during days 0-10 in this protocol, even when MATRIGEL™ is present during days 10-20, does not stimulate Pdx-1 expression. This experiment also emphasizes the role of the Activin/BMP4 co-stimulation during this early window of differentiation.

### Example 6

### Directed Differentiation of Mouse Embryonic Stem Cell Reporter Lines

A mouse embryonic stem cell line with a lacZ reporter knocked into the pdx-1 locus was differentiated along the pancreatic lineage. Cultures of ES cells were used to generate EBs which were subjected to culture in the presence of early and late factors. FIG. 4A shows a cluster of cells expressing β-galactosidase, thus indicating expression of pdx-1, after EB formation and subsequent plating. FIG. 4B shows quantitative RT-PCR data for pdx-1 for mouse EBs at various stages of culture. It is apparent that pdx-1 expression increased over time up to 24 days of EB formation.

### Example 7

### Directed Differentiation Along a Pancreatic Lineage Increases Over Time

Human embryonic stem cell line hES2 was used to generate EBs suspended in MATRIGEL™. Cell were treated with the early and late factors, as described above. Expression of pdx-1 in these EBs derived increased with time. FIG. 5A shows that pdx-1 mRNA, as detected by real time RT-PCR, increased with the number of days of EB formation between 0 to 24 days. FIG. 5B shows an ethidium bromide stained gel of the pdx-1 RT-PCR product, indicating that a single band of the predicted size was detected.

### Example 8

### Various Growth Factor Preparations Promote Directed Differentiation Along a Pancreatic Lineage

Human embryonic stem cell line hES3 was used to generate EBs suspended in MATRIGEL™. Addition of TGF-β factors increased pdx-1 expression in hES3. hES3 EBs were cultured in MATRIGEL™ in RPMI media with addition of several TGF-β related factors exemplified by Activin, BMP-2, BMP4 or Nodal. Expression of pdx-1 by RT-PCR was measure after 20 days in culture. As shown in FIG. 6, expression is expressed as fg per ng actin. Addition of growth factors led to a 9-fold increase in pdx-1 expression in comparison to culture in the absence of growth factors. Furthermore, this treatment resulted in an increase in insulin expression, as measured by RT-PCR, after 20 days in culture. Expression is expressed as fg per ng actin. Addition of growth factors led to about a 2-fold increase in insulin expression.

### Example 9

### Directed Differentiation of Human Embryonic Stem Cells Along Particular Endodermal Lineages

The methods of the invention can be used to direct the differentiation of stem cells to particular endodermal cell types. The results summarized in FIG. 7 demonstrate that hepatocyte cell types can be differentiated from embryonic stem cells. FIG. 7A shows expression of the hepatocyte marker albumin in hES cells cultured by directly plating ES cells on MATRIGEL™ coated tissue culture plates.

FIG. 7B shows quantitative RT-PCR data for two different hES lines subjected to several differentiation protocols. Human ES cells differentiated according to condition E had the largest increase in expression of several hepatic markers. ES cells were plated on MATRIGEL™ coated plates and maintained in knock-out media supplemented with 20% serum replacement and 1% DMSO. After five days, cells were treated with 2.5 mM sodium butyrate. The medium was then replaced with hES media supplemented with 100 ng/ml alpha-FGF, 0.1 ng/ml TGF-β, 30 ng/ml EGF, and 30 ng/ml HGF. Finally, the medium was replaced with hES media supplemented with 10 ng/ml oncostatin M, 1 μM dexamethasone, 30 ng/ml HGF, and 0.1 ng/ml TGF-β.

### Example 10

### Directed Differentiation of Human Embryonic Stem Cells Along a Pancreatic Lineage Using a Combinatorial Approach—44 Day Protocol

As outlined in detail above, the differentiation of human embryonic stem cells grown in 3-dimensional culture can be directed along the pancreatic lineage, as indicated by expression of pdx-1. Additional experiments were then conducted to see whether the directed differentiation of human embryonic stem cells along pancreatic lineage can be further influenced by subjecting the cells to a combination of the 3-D culture system outlined above and other culture systems shown by us to influence differentiation of cells to pancreatic cell types.

To establish a baseline for comparison, human embryonic stem cells were differentiated, as described above, in 3-dimensional culture in MATRIGEL™ for 20 days. For the first 10 days the cells were cultured in the presence of early factors (Activin A: 50 ng/ml; BMP2: 50 ng/ml; BMP4: 50 ng/ml; Nodal: 50 ng/ml) and for the second 10 days the cells were cultured in the presence of late factors (Betacellulin: 50 ng/ml; HGF: 50 ng/ml; Exendin-4: 10 ng/ml, Nicotinamide: 10 mM). pdx-1 levels were measured by RT-PCR at various time points during culture of the cells in the 3D MATRIGEL™ protocol. RNA was isolated and analyzed for pdx-1 and actin expression by RT-PCR. Data were standardized in comparison to actin expression, and the results are expressed as absolute g pdx-1/kg Actin expression+/−SD. These results are summarized in FIG. 8.

We then set up an experiment based on combining the above MATRIGEL™, 3-dimensional culture protocol with a 24 day, 5 step differentiation protocol originally evaluated for its ability to produce insulin⁺ cells from pancreatic ductal precursors. Human embryonic stem cells were either placed directly into the 24 day protocol, or were first cultured for 1, 2, or 3 weeks in the 3 dimensional MATRIGEL™ protocol outlined above.

Method: Day 20 was considered the first day of the 24 day protocol. Prior to that, cells were cultured in 3D culture in MATRIGEL™. The methodology used for culturing cells that were first cultured for the full 3 weeks in 3D culture and then subject to the 24 day differentiation protocol is as follows:

D0-10 (Days 0-10): Day 0 is normally the set up day. Cells were cultured in KO SR medium+early growth factor cocktail (Activin A: 50 ng/ml; BMP2: 50 ng/ml; BMP4: 50 ng/ml; Nodal: 50 ng/ml). The medium was topped up (to feed the cells) at D 3, 6.

D10-20 (Days 10-20): Cells were cultured in KO SR medium+late growth factor cocktail (Betacellulin: 50 ng/ml; HGF: 50 ng/ml; Exendin-4: 10 ng/ml; Nicotinamide: 10 mM). The medium was changed at 16.

D20: EBs were eluted from 3D MATRIGEL™ and re-plated on low attachment plates.

D20-26 (Days 20-26): Begin 24 day maturation protocol=Step 1 of 24 day protocol. Cells were cultured in basal medium for 6 days (DMEM/F12, 17 mM Glc, 2 mM Glutamax, 8 mM HEPES, 2% B27, +Penicillin/Streptomycin). Cell were fed with fresh media on day 23.

D26 (Day 26): EBs were dissociated by Dispase (1 of 2 wells, the other one remains as EB) and re-plated on low attachment plates.

D26-32 (Days 26-32): Step 2 of 24 day protocol. Cells were cultured in basal medium+20 ng/ml FGF-18, 2 μg/ml Heparin (new medium at D26, new GFs added at D29).

D32-36 (Days 32-36): Step 3 of 24 day protocol. Cells were cultured in basal medium+20 ng/ml FGF-18, 2 μg/ml Heparin, 10 ng/ml EGF, 4 ng/ml TGFα, 30 ng/ml IGFI, 30 ng/ml IGFII, 10 ng/ml VEGF (new medium at D32, new GFs added at D34).

D36 (Day 36): Cells were re-plated on Fibronectin-coated plates.

D36-40 (Days 36-40): Step 4 of 24 day protocol. Cells were cultured in RPMI medium (11 mM Glc, 5% FBS, 2 mM Glutamax, 8 mM HEPES, Penicillin/Streptomycin)+10 μM Forskolin, 40 ng/ml HGF, 200 ng/ml PYY (new medium at D36, new GFs added at D38).

D40-44 (Day 4044): Step 5 of 24 day protocol. Cells were cultured in CMRL medium (5 mM Glc, 5% FBS, 2 mM Glutamax, Penicillin/Streptomycin)+100 ng/ml Exendin-4, 5 mM Nicotinamide (new medium at D40, new GFs added at D42).

D44 (Day 44). RNA was harvested for analysis of pdx-1 and insulin expression by RT-PCR.

Note: RNA samples were harvested from cells at various points along this process to help evaluate the directed differentiation of the cells.

Results: The 24 day protocol contains a step where the cells are can be dissociated using dispase. In our experiments, we assessed whether this dispase dissociation step was necessary, and whether it negatively impacted the ability of cells to be differentiated along a pancreatic lineage.

As outlined above, following the standard 3D differentiation protocol, embryonic stem cells differentiate to express a high level of pdx-1. As shown in FIG. 9A, cells subject to both the 20 day 3D differentiation protocol and the 24 day protocol continue to express pdx-1. However, pdx-1 expression is at a lower level than following the first 20 days in culture (compare FIG. 8 and FIG. 9A). Additionally, however, these cells express very high levels of insulin mRNA (See, FIG. 9B). Expression of insulin protein will be confirmed by performing a C-peptide ELISA assay.

Furthermore, as shown in both FIG. 9A and FIG. 9B, treatment of the cells with dispase during the 24 day differentiation protocol had a negative impact on the ability of these cells to differentiate along a pancreatic lineage. Thus, elimination of this step may be useful.

### Example 11

### Directed Differentiation of Human Embryonic Stem Cells Along a Pancreatic Lineage Using a Combinatorial Approach—34 Day Protocol

As outlined in detail above, the differentiation of human embryonic stem cells grown in 3-dimensional culture can be directed along the pancreatic lineage, as indicated by expression of pdx-1. Additional experiments were then conducted to see whether the directed differentiation of human embryonic stem cells along the pancreatic lineage can be further influenced by subjecting the cells to a combination of the 3D culture system outlined above and other culture systems shown by us to influence differentiation of stem cells. One such other culture system was the 24 day protocol outlined in Example 10. We additionally tested a 34 day differentiation protocol.

In this combinatorial approach, cells are subjected to culture and differentiation in 3D culture for only 10 days. At that point, cells are than taken and subjected to a 34 day differentiation protocol. Following this combinatorial approach, expression of pdx-1 and insulin was assessed by RT-PCR.

Method: This protocol from start to finish is 34 days long. Prior to that, cells were cultured in 3D culture in MATRIGEL™ for 10 days in the presence of the early growth factor cocktail. The methodology for cells that were first cultured for 1 week in 3D culture and then subject to the 34 day differentiation protocol is as follows:

D1-10 (Days 1-10): Cells were cultured in KO SR medium+early growth factor cocktail (Activin A: 50 ng/ml; BMP4: 50 ng/ml). The medium was change at D1, 3, 6.

D10 (Day 10): EBs were eluted from 3D MATRIGEL™ and re-plated on low attachment plates.

D10-18 (Days 10-18): Step 2 of 34 day protocol. Cells were cultured in basal medium ((DMEM/F12, 17 nM Glucose, 2 nM Glutamine, 8 mM HEPES, 2% B27 and Pen/strep)+20 ng/ml FGF-18, 2 μg/ml Heparin, The cells were fed on day 13 and 16 with media plus growth factor top ups.

D18-24 (Days 18-24): Step 3 of 34 day protocol. Cells were cultured in basal medium+20 ng/ml FGF-18, 2 μg/ml Heparin, 10 ng/ml EGF, 4 ng/ml TGFα, 30 ng/ml IGFI, 30 ng/ml IGFII, 10 ng/ml VEGF. The cells were fed fresh medium and growth factors on Day 21.

D24 (Day 24): Cells re-plated on Fibronectin-coated plates.

D24-29 (Days 24-29): Step 4 of 34 day protocol. Cells were cultured in RPMI medium (11 mM Glc, 5% FBS, 2 mM Glutamax, 8 mM HEPES, Penicillin/Streptomycin)+10 μM Forskolin, 40 ng/ml HGF, 200 ng/ml PYY. The cells were fed on Day 27 with fresh media and growth factors.

D29-34 (Days 29-34): Step 5 of 32 day protocol. Cells were cultured in CMRL medium (5 mM Glc, 5% FBS, 2 mM Glutamax, Penicillin/Streptomycin)+100 ng/ml Exendin-4, 5 mM Nicotinamide. The cells were fed fresh medium and growth factors on Day 32.

D34: Continue culturing cells in CMRL w/o growth factors and harvest the cells.

Note: RNA samples were harvested from cells at various points along this process to help evaluate the directed differentiation of the cells. Furthermore, culture medium and factors were regularly changed throughout the differentiation protocol.

Results: As outlined in Example 10 above, following the standard 3D differentiation protocol, embryonic stem cells differentiate to express a high level of pdx-1. We additionally directed the differentiation of embryonic stem cells along a pancreatic lineage using the approach outlined in Example 11. During the protocol outlined in Example 11, we harvested samples at various points during the differentiation protocol. FIG. 10 summarizes the results for two time points: day 10 (just prior to beginning the 34 day protocol) and day 22 (approximately ⅓ of the way through the 34 day differentiation protocol).

FIGS. 10A and 10B show that at day 10 (prior to beginning the 34 day protocol), expression of pdx-1 is low and expression of insulin is undetectable. However, as shown in FIGS. 10A and 10B, at 22 days the expression of pdx-1 is very high. In fact, expression of pdx-1 at this point is higher than after completion of the 24 day protocol (see, FIG. 9A). As shown in FIG. 10B, at 22 days insulin expression can be detected. However, expression of insulin at this point is not as robust as following the completion of the 24-day protocol (see, FIG. 9B). This may indicate that part way through the 34-day differentiation protocol, the cells are continuing to terminally differentiate along the pancreatic lineage. At approximately 22 days, expression of pdx-1 is still relatively high, perhaps indicating that more cells are capable of but have not yet differentiated to insulin expressing cells.

Analysis of cells using similar methods at various time points in the 34-day differentiation protocol can refine the time and conditions under which cells are directed to partially versus terminally differentiate along a pancreatic lineage.

### Example 12

### Directed Differentiation of Embryonic Stem Cells to Pdx-1⁺ Cells Using the 20-Day Differentiation Protocol Mimics Normal Pancreatic Development

Much of the work aimed at the in vitro generation of beta cells from embryonic and adult stem cells has focused on the activation of the pdx-1 gene due to its tissue-specific expression in early pancreatic progenitors. However, one potential criticism of a reliance on pdx-1 expression as indicative of differentiation along a pancreatic lineage is that the particular in vitro differentiation scheme used may result in spurious activation of pdr-1. Such spurious activation of pdr-1 may not indicate differentiation along a pancreatic lineage, and may not provide a good predictor of cells capable of further differentiation to insulin expressing, glucose responsive cells. Accordingly, we conducted experiments designed to demonstrate whether that pdx-1 expression during the directed differentiation of embryonic stem cells using our protocols is physiologically relevant to normal pancreatic development.

We performed an expression time course of genes that are normally activated during formation of the definitive endoderm. As shown in FIGS. 11 and 12, the in vitro expression kinetics of these markers roughly followed the expected in vivo activation sequence of gene expression during normal beta cell development. The rapid increase in Brachyury (Thra) (FIG. 11) accompanies a drop in the pluripotency markers Oct4 and nanog (FIG. 12) and suggests the start of gastrulation-related processes and formation of the embryonic germ layers. This is followed by a more sustained expression of the endodermal genes Hnf3β and Sox17 that precedes the emergence of pdx-1-expressing cells beginning on day 15. On day 20, insulin transcripts were detected, with levels increasing with extended differentiation. Interestingly, Oct4 expression levels were up-regulated at day 20 to around 60% of undifferentiated levels (day 0). This could be due to the emergence of other Oct4-expressing cell types such as primordial germ cells. In addition, a cursory examination of other mature lineage markers such as albumin, AFP, and Cyp3A4, normally expressed in forming liver cells, revealed an early expression peak followed by a general decrease (FIG. 12).

This analysis indicated that the in vitro methods of the invention for directing the differentiation of embryonic stem cells along a pancreatic lineage induced proper gene expression in a temporally regulated fashion that mimics that observed during normal pancreatic and beta islet cell differentiation.

### Example 13

### Further Optimization of the 20-Day Differentiation Protocol

As outlined in detail above, we have developed a 20-day differentiation protocol (initiation protocol) that directs the differentiation of embryonic stem cells along a pancreatic lineage. Specifically, we have developed a protocol involving addition of early factors and late factors that direct the differentiation of embryonic stem cells to pdx-1⁺ cells that can further differentiate to insulin+cells. Although the above early/late factor (EF:LF) differentiation protocol is effective, we conducted additional experiments designed to further optimize this methodology.

We evaluated individual early and late factors. These studies indicated that, of the 8 factors (4 EF and 4 LF) proven effective in our initiation protocol, BMP4 and Activin A were important components of the early growth factor mix. The efficacy of BMP4 and Activin A was most dramatic when the late factor mix excluded the poly(ADP-ribose) polymerase inhibitor Nicotinamide. These studies indicated that an initial differentiation protocol based on only 2 early factors and only 3 late factors (rather than 4 early factors and 4 late factors) was highly effective and can be can be readily used to promote the directed differentiation of embryonic stem cells to pdx-1⁺ cells biased to differentiation along a pancreatic lineage. This revised protocol differs from the initial 20-day protocol only in the nature of the early and late factors used. This 2 EF-3 LF protocol included the early factors Activin A (50 ng/ml) and BMP-4 (50 ng/ml). The cells were cultured in the early factors as previously described from day 0 to day 10. The 2 EF-3 LF protocol included the late factors HGF (50 ng/ml), exendin-4 (10 ng/ml), and β-cellulin (50 ng/ml).

The 2 EF-3 LF protocol represents an improvement over the previous 4 EF-4 LF protocol because it induced robust pdx-1 expression using a cheaper, faster, and simpler procedure. FIGS. 13-16 summarize the experiments that led to the development of the 2 EF-3 LF protocol.

Without being bound by theory, since BMP2 and BMP4 are two structurally-related growth factors that bind the same cell surface receptor complex, it was anticipated that the combination of the two growth factors was super-saturating in the 4 EF mix and that one factor alone would be sufficient for pdx-1 induction. FIG. 13A summarizes data showing that an EF growth cocktail lacking BMP2 induced pdx-1 expression at levels slightly greater than that induced by all four early EFs (compare G2 to G7 in FIG. 13A). Somewhat surprisingly, the TGF-β-related ligand Nodal, which is an evolutionarily conserved endoderm inducer, had little effect in the EF cocktail. Note, in FIG. 13, “All” indicates the addition of all four of the EF and/or LF used in the initial 4 EF4 LF 20-day protocol. Duplicate wells for each experimental condition are shown along with Ct value Pdx-1/actin of the best performing conditions. Note that the results depicted in FIG. 13A represent normalized expression, and the resulted depicted in FIG. 13B represent Pdx-1 expression as % actin input.

Additional experiments demonstrating that Nodal does not significantly improve induction of pdx-1 expression are summarized in FIG. 14. We note, however, that Nodal did not appear to have any adverse effect on pancreatic differentiation, and thus could optionally be included in the initiation protocol. Briefly, the 2 EF-3 LF protocol was performed in the presence or absence of 50 ng/ml recombinant Nodal. On day 20, samples were analyzed by Q-PCR for pdx-1 expression, and calculated against a Pdx-1 and actin standard curve. Students T-test established the absence of statistical significance (p>0.05) between the two experimental conditions.

Without being bound by theory, one explanation for this result is that differentiating human ES cells express low levels of Cripto, the requisite co-receptor for Nodal, and that in our assay, Activin protein mimics the endogenous Nodal signal. This hypothesis prompted the removal of BMP2 and Nodal from the early factor mix. Further analysis revealed that the remaining two EFs (BMP4 and Activin A) in combination yielded pdx-1 expression levels far greater than those initially observed with the 4 EF-4 LF mix (compare G2 and G8 in FIG. 13A). The synergistic effect of BMP4 and Activin is further supported by single factor experiments (compare G8 through G11 in FIGS. 13B and G2 through G7 in FIG. 15).

In addition, many experiments were performed to determine the optimal concentration of BMP4 and Activin in combination. We repeatedly found that 50 ng/ml BMP4 and 50 ng/ml Activin together with the LF mix lacking nicotinamide induced robust pdx-1 expression in addition to other markers of the endocrine lineage including insulin, glucagon, Pax4 and somatostatin (FIGS. 15 and 16).

As mentioned above, these experiments indicated that the presence of nicotinamide in the LF mix inhibited pdx-1 expression both in context of the 4 EF (compare lanes G2 with G5 and G6 in FIG. 13A) and the streamlined 2 EF mixes (compare G3 through G5 in FIG. 13B). Thus, although pdx-1 expression was achieved using the 4 EF-4 LF initiation protocol that included nicotinamide, this factor was omitted in the 2 EF-3 LF initiation protocol.

In contrast, these experiments indicated that Beta-cellulin was an important component of the LF mix (compare G2 with G5 and G6 in FIG. 13A, and G3 through G5 in FIG. 13B). Accordingly, Beta-cellulin was retained as a LF in the 2 EF-3 LF initiation protocol.

Further, the importance of LFs HGF, exendin-4, and beta-cellulin on pdx-1 expression by pancreatic cells was investigated. During differentiation in MATRIGEL™, low levels of pdx-1 are first detected as early as 12 days (See, for example, FIGS. 4B, 11, 12) and increase gradually over time. The growth factors, HGF, exendin-4 and beta-cellulin, have been extensively characterized for their roles in the maturation, proliferation or modulation of the insulin-secreting kinetics of more specialized islet-derived cell populations, and are thus predicted to provide little instructive signaling to the emerging pdx-1-expressing pancreatic progenitors. The contribution of each of the 3 LF as well as nicotinamide toward the expression of pdx-1 on day 20 was assessed. The results are shown in FIG. 29, indicating the removal of the 3LF leads to roughly a 3-fold increase in pdx-1 levels (second column from the left). These data suggest that the combination of the EFs, Activin A and BMP4, is sufficient to launch pancreatic differentiation within the 3D MATRIGEL™ matrix. No pdx-1 expression was detected either in the no growth factor control or the 3LF alone (administered beginning on day 10). In FIG. 29, Ex-4: exendin-4; Nic: nicotinamide; HGF: hepatocyte growth factor; β-cell: beta-cellulin; 4LF: 3LF plus nicotinamide.

In summary, these experiments demonstrated that multiple initiation protocols can be used to help direct the differentiation of embryonic stem cells along a pancreatic lineage. Two representative examples of these initiation protocols are the 4 EF-4 LF initiation protocol and the 2 EF-3 LF initiation protocol described herein. Other protocols in view of these two examples are within the scope of the invention.

The results shown in FIG. 29 was used to develop the simplified maturation protocol described in Example 16, which removed steps 1 and 5 from the standard protocol.

### Example 14

### Localization of Pdx-1 Expressing Cells Following Directed Differentiation of Embryoid Bodies via the Initiation Protocol

Pdx-1 immunohistochemistry was performed to corroborate the Q-PCR (quantitative-PCR) data presented above and to allow localization and quantification of Pdx-1-expressing cells within EBs. This procedure (described at length in the Material and Methods) has been repeated on EBs generated from numerous independent differentiation experiments to eliminate the possibility of artifactual staining. Because the initiation protocol experiments (described above in Example 13) relied on the induction of Pdx-1 mRNA expression as assessed by Q-PCR, it was important to exclude the possibility that BMP2, Nodal and/or nicotinamide negatively effect the production and accumulation of Pdx-1 protein.

FIG. 17 shows immuno-localization of Pdx-1 in embryoid bodies differentiated using the initiation protocol. EBs were differentiated for 20 days using either the 4 EF4 LF protocol (FIGS. 17A and 17B) or the 2 EF-3 LF protocol (FIGS. 17C and 17D). Using either initiation protocol, Pdx-1-positive cell populations were identified within epithelial ribbons that often enclose lumens and are often confined to the EB periphery (arrows in FIGS. 17A and 17B). Without being bound by theory, the clusters of Pdx-1-expressing cells may suggest that a subpopulation of EBs support a pancreatic “niche” that underlies the further development of insulin-producing cells.

The expression of pdx-1 mRNA was further analyzed by in situ hybridization (FIG. 22), and shown to precisely correlate with Pdx-1 immunohistochemistry. Briefly, FIGS. 22A and 22B show pdx-1 expression by in situ hybridization after the 20-day initiation protocol (2 EF-3 LF). The results summarized in FIGS. 22A and 22B indicated that approximately ⅓ of all EBs harvested from an individual culture well express Pdx-1 (darker staining) after 20 days of differentiation. The higher magnification view shown in FIG. 22B demonstrates that pdx-1 transcripts localized near the periphery of the EB. FIG. 22C indicated that EBs cultured in the absence of growth factors fail to express Pdx-1. FIG. 22D summarizes the results of quantitative PCR of parallel cultures of those cells shown in FIGS. 22A and 22C, and confirms robust pdx-1 expression in cultures containing growth factors (higher bar) versus those differentiated in the absence of growth factor (lower bar).

Additionally, light hematoxylin counter-staining was used in combination with section immunohistochemistry to estimate the number of pdx-1-expressing cells in a group of EBs. A field of EBs (a total of 65) was broken into smaller regions for manual counting. Total cell number was determined by hematoxylin staining. We found that at day 20, approximately 1% of the cells per section were Pdx-1-positive. In a field of EBs, around ⅓ of the EBs contained Pdx-1-positive clusters.

The expression pattern of pdx-1 in the EBs was further investigated by exploring whether Pdx-1 and C-peptide expressions co-localize. The hES3 cells were directed to differentiate using 2EF-3LF initiation protocol, and a simplified maturation protocol using simply Step 4, as further described in Example 15, “Step-4 only maturation.” Sections of the resulting EBs were prepared and immuno-stained using antibodies to Pdx-1 and C-peptide, either as a single or double stained immunohistochemistry samples. The results are shown as FIG. 28, wherein the top panels show the high magnification images and the bottom panels show lower magnification images, with DAPI-stained nuclei. FIG. 28 shows that Pdx-1-positive cells localized in clusters or epithelial ribbons of the differentiated hES3 cells, and there are C-peptide positive cells among them.

### Example 15

### Maturation Protocols

Five-Step Maturation

The initiation protocols provide a straightforward differentiation regime that directs pluripotent embryonic stem cells toward pdx-1-expressing pancreatic progenitors. At the end of the 20-day differentiation protocol (e.g., the initiation protocol), insulin expression is still relatively low. Thus, we used a maturation protocol to promote further pancreatic differentiation of the biased cells.

FIG. 18A provides a schematic representation of a particular combination of the 2 EF-3 LF initiation protocol plus a maturation protocol. The maturation protocol depicted in FIG. 18A may have as many as 5 steps. The use of any combination of these steps will be referred to as a maturation protocol, and reference to the stage/step, the number of days in culture, and/or the particular factors used will distinguish permutations of the maturation protocol.

Briefly, samples differentiated using the 2 EF-3 LF initiation protocol were further differentiated using this 5 step, 24 day maturation protocol. There was a strong correlation between pdx-1 expression levels at day 20 (FIG. 18B, left panel) and the release of C-peptide into the medium after stage 3 of the maturation protocol (day 36, FIG. 18B right panel). Note that C-peptide is the stable by-product released during enzymatic processing of proinsulin, and provides a indirect but reliable measure of insulin secretion in our assays. This finding support the ideas (1) that mid-point (day-20) pdx-1 expression presages the later emergence of more mature cell types and (2) that the combined in vitro protocol approximates the developmental cues that guide pancreatic progenitors toward terminally differentiated endocrine cells.

As shown in FIG. 18B (right panel), we detected C-peptide release into the medium after a total of 36 days of extended in vitro culture. We began a systematic investigation of the relevance of the five steps of the maturation protocol shown schematically in FIG. 18A. We first devised a series of simple process-of-elimination experiments aimed at investigating each of the steps. Day 20 pdx-1-expressing EBs from the 2 EF-3 LF differentiation protocol were directly shunted into only one step of the multi-step protocol for an additional 24 days of differentiation. In these experiment, the baseline concentration of C-peptide detected after 48 hours of culture (again on day 36) was approximately 0.5 ng/ml, as measured in control cultures that have progressed through the first four steps of the maturation protocol—upper schematics—FIGS. 18A and 19A.

FIG. 19B summarizes the results of these experiments. Interestingly, there was little difference in C-peptide release on day 36 for each of the other conditions aside from Stage 4, which showed a 6-fold increase in C-peptide release (FIG. 19B). There are many unique aspects to this particular stage: most notably is the continued use of RPMI as the base media, and growth on fibronectin-coated dishes.

Step-4 Only Maturation

The maturation protocol was further refined as shown in the diagrams of FIG. 23. The top diagram is the original 5 step maturation protocol. The middle diagram shows using only Step 4 of the 5-step protocol. Twenty-day old MATRIGEL™ EBs were washed with cold PBS to remove excess MATRIGEL™, and replated onto fibronectin-coated dishes and cultured directly in Step 4 medium for 4 days. Steps 1-3, as well as Step 5 were omitted.

This simplified protocol was developed based on the observation that Step 4 of the muti-step protocol is the key step to the release of C-peptide from differentiating hES3 cells. As shown in FIG. 24, various permutations of the maturation steps were investigated. The arrows at the bottom of the graph show time points when the culture medium was either changed or maintained, according to the permutations shown at the top of the graph. In all variations, a spike in C-peptide release is observed in cultures when they transition to Step 4 culture medium. In fact, in cultures that were shunted from the end of the 20-day MATRIGEL™ protocol directly into Step 4 medium (dashed line in the graph of FIG. 24), C-peptide release was accelerated, with a measurable amount released by day 27, and was sustained over the 45-day culture period.

FIG. 24 also shows that Step 5 consistently made the C-peptide level to decrease, and the culture exposed only to Step 5 medium showed no C-peptide release (black line).

Maturation Factor Investigation

The active maturation component of Step 4 was investigated using the simplified Step 4-only protocol, wherein the differentiating EBs were shunted directly into Step 4 culture conditions after the 20-day MATRIGEL™ protocol. Step 4 medium was modified by removing some of the components, and conditioned medium was collected on day 30 from cultures grown in the modified Step 4 medium. As shown in the diagrams of FIG. 23, Step 4 medium is based on RPMI and normally contains 5% FBS, 10 μg forskolin, 40 ng/ml HGF, and 200 ng/ml PYY. The result of the experiment is shown in FIG. 25. The removal of all additional growth factors and forskolin, while retaining 5% FBS, causes an approximately two-fold decrease in the levels of C-peptide in the culture medium. Adding back each of the removed components individually or in combination shows that forskolin contributes to the release of C-peptide (See FIG. 25A). The analysis of the aggregate of the experimental results shown in FIG. 25A comparing all forskolin-containing conditions and all forskolin-lacking conditions reveals a statistically significant increase in C-peptide release when forskolin is present in the medium. Similar effects are seen on insulin expression (FIG. 25B).

In contrast to forskolin, FBS was shown to be dispensable and not an essential component of Step 4 medium. Briefly, hES3 cells were differentiated using the simplified maturation protocol described above. Step 4 medium was prepared using either RPMI following the standard protocol or CMRL, supplemented with FBS, a commercially available serum replacer (SR) which is chemically defined, or not supplemented at all. C-peptide levels were measured on Day 28. As shown in FIG. 26A, there was no statistically significant difference in C-peptide release between culture using medium based on RPMI or CMRL. Further, as shown in FIG. 26B, FBS (center bar) can be omitted from the medium (left bar), and more than adequately compensated by the SR (right bar), for the release of C-peptide.

It was further demonstrated that a low concentration of glucose in the medium abolishes the release of C-peptide and significantly decreases insulin mRNA level in the differentiated hES3 cell population, indicating that the differentiated hES3 cell populations contain cell types capable of glucose-stimulated insulin/C-peptide release. Specifically, hES3 cells were differentiated using the simplified maturation protocol described above. On day 30 of the protocol, the culture medium was removed and replaced by either RPMI or DMEM supplemented by 22 mM glucose, RPMI supplemented with 22 mM glucose, or DMEM supplemented by 5 mM glucose. 5 mM glucose mirrors the physiological concentrations of glucose at which insulin is stockpiled in secretory granules. After 48 hours in the replaced media, C-peptide levels in conditioned medium were assessed by ELISA. As shown in FIG. 27, there was little or no statistically significant difference between the media containing 22 mM or 11 mM of glucose, but C-peptide levels were unexpectedly reduced to nearly undetectable levels in the conditioned DMEM with 5 mM glucose (FIG. 27B). Similarly, insulin mRNA expression remains unchanged at 11 mM and 22 mM glucose concentration, but drops significantly at 5 mM glucose (FIG. 27D). In contrast, the expression level of pdx-1 mRNA is not significantly affected (FIG. 27C). The result is consistent with the well-characterized regulation of insulin gene expression by glucose, and indicated the presence of differentiated cells that are responsive to glucose stimulated regulation of insulin.

### Example 16

### Maturation Protocol Following Modified 10 day Initiation Protocol

We cultured EBs in a modified initiation protocol that included only the first 10 days EF phase of the protocol, but omitted the second 10 day LF phase of the protocol. EBs cultured in this manner were then subjected to steps 2 through 4 of the maturation protocol. Cells differentiated in this manner robustly secreted C-peptide (as much as 12-14 ng/ml). Note that in Examples 15 and 16, C-peptide release was assayed by ELISA.

The modified initiation protocol was also used in a simplified multi-step maturation protocol shown at the bottom of FIG. 23. Briefly, 10-day old MATRIGEL™ EBs were washed free of MATRIGEL™ with cold PBS and then replated in suspension culture in Step 2 medium for 8 days, followed by Steps 3 (6 days) and 4 (5 days).

When conditioned medium was sampled on days 26 and 29, some amount of C-peptide was consistently detected (See FIG. 31).

### Example 17

### Cellular Characterization of Pancreatic Cell Types Following Differentiation Using the Initiation and/or Maturation Protocols

We investigated the distribution and number of insulin/C-peptide synthesizing cells per EB at different time points in the differentiation regime. A full-length insulin antisense riboprobe was generated for whole-mount in situ hybridization (WISH), a technique that permits the identification of individual cells expressing insulin mRNA in 3D cultures. Consistent with the low levels of insulin detected by Q-PCR at the end of the 20-day 2 EF-3 LF protocol, WISH reveals very few insulin-expressing cell clusters on the surface of individual EBs at this stage (FIGS. 20A and 20B). At this stage, based on inspection of cryo-sectioned material (FIG. 20E), we estimated that each individual cluster contains at most 4 to 5 cells. In contrast, further differentiation using the maturation protocol stimulated the expansion and formation of numerous sharply defined insulin-expressing cell clusters (FIG. 20C), with some EBs showing intense surface staining in enlarged patches (higher magnification in FIG. 20D) while others show little to no staining. Interestingly, we found that the majority of small to medium-sized EBs contained at least a few insulin-expressing clusters that are roughly confined to one region of an EB, suggesting both the formation of a pancreatic niche and an EB size for which there exists a propensity for the further maturation of insulin-producing islet-like cells.

These data are further corroborated by C-peptide section immunohistochemistry (FIG. 21). Briefly, FIG. 21 shows paraffin sections of day 45 EBs following immunocytochemistry with C-peptide. C-peptide-positive cells were often distributed at the EB periphery in a manner similar to insulin-expressing cell clusters, but were sometimes located in more interior regions (FIG. 21B-F). FIG. 21A shows, as a positive control, C-peptide immunolocalization in an adult mouse islet.

Double labeling experiments in day 45 EBs revealed co-localization of insulin and C-peptide in a punctuate pattern that is strikingly reminiscent of secretory granule storage in endogenous beta cells. This provides strong evidence that the insulin-expressing cells induced using the combination of a initiation and maturation protocol stockpile insulin and C-peptide peptides for glucose-stimulated secretion, thus mimicking a biochemical property of endogenous, normal beta cells.

Similar co-localization can be seen when the EBs subject to the simplified protocol without Steps 1 and 5 were immunofluorescent stained. FIG. 32 shows high magnification (top panels) and low magnification (bottom panels) images of paraffin-embedded and sectioned EBs harvested on Day 26 from the simplified protocol. At this stage, most insulin positive cells were also reactive for C-peptide, which is the by-product of insulin synthesis. Nuclei are shown by the staining by DAPI.

FIG. 33 shows additional evidence of differentiation through the simplified protocol without standard protocol Steps (1) and (5). When EBs subject to the simplified protocol were examined for Nkx6.1 and Pdx-1 immunoreactivity, the immunofluorescent stain showed efficient formation of cells belonging to the beta-cell lineage (left panels: top, high magnification; bottom, low magnification). These cells were largely confined to epithelial ribbons or tubes that enclose luminal spaces. In addition, Pdx-1-positive cells clusters are also reactive for glucose transporter Glut2, as can be seen in the right panels. Nuclei are shown by the staining by DAPI.

Unless otherwise specified, the following methods were used for the experiments outlined in Examples 12-17.

Human ES cell culture: hES cells were cultured according to standard procedures. The generation of embryoid bodies (EBs), and the culture of the cells within the 3D MATRIGEL™ during the first 20 days were as follows:

Part 1: Collagenase Treatment of hES1-6

1. discard central button and cystic parts of the hES colonies under dissecting microscope using glass pipette pump suction. Wash hES plate (P100 tissue culture plate) with PBS twice. Note: This suction step can be done either before or after collagenase treatment.

2. Add 3 ml of 1 mg/ml collagenase IV to the hES plate, and keep the plate in 37° C. CO₂ incubator for 8 min.

3. Aspirate collagenase solution.

4. Wash with 10 ml of PBS once. Aspirate PBS gently. Do not disturb colonies.

5. Add 10 ml of the RPMI/20SR to the plate.

6. Mechanically dissect the hES plate using 2-ml Pasteur pipette.

7. Use cell scraper to dislodge all the dissected pieces.

8. Pipette up and down for a few times to resuspend pellets.

9. Transfer pellets to a 15-ml falcon tube.

10. Wash plate once more with RPMI/20SR to get all the residue pellets from the plate.

11. Transfer pellets to the 15-ml falcon tube.

12. Spin down the pellets at 1500 rpm, 4 min.

13. Aspirate supernatant medium.

Part 2: Make MATRIGEL™ EB

Early Factor Stage

Day 0

1. Pre-chill medium on ice or 4° C. Pre-chill 2 ml, 5 ml and 10 ml pipettes at −20° C.

2. Prepare 1:6 MATRIGEL™ medium (e.g., 1 ml liquefied MATRIGEL™+5 ml RPMI/20SR) using pre-chilled pipettes. Total volume is according to 2 ml for each well.

3. Resuspend hES pellets in MATRIGEL™ medium.

4. Add 2 ml of hES pellet suspension in one well of Ultra-low plate. Note: One P100 plate of hES colonies can be split into 3 to 5 wells depending on the confluence.

5. For the growth factor group, growth factor cocktail (100 ng Activin A+100 ng BMP4 per well) can either be added in the MATRIGEL™ medium before pellet resuspension procedure or directly to the well immediately after cell suspension is plated into the Ultra-low plate.

6. Keep Ultra-low plate in 37° C. cell incubator. MATRIGEL™ medium will gel after several hours. And hES pellets will round up and form embedded EBs after overnight.

Day 3 and 6

7. Top up with RPMI/20SR (0.5 ml)+EF (same amount as 1^{st} dose−100 ng Activin A+100 ng BMP4 per well).

Late Factor Stage

D10

1. Remove medium with 1-ml blue pipette under dissecting microscope very slowly and carefully. Do not suck away MATRIGEL™ and EBs.

2. Add 3 ml fresh RPMI/20SR to each well and equilibrate for 1 hr in cell incubator.

3. Remove 3 ml medium with 1-ml blue pipette under dissecting microscope very slowly and carefully.

4. Top up with RPMI/20SR (0.5 ml)+LF (100 ng HGF+20 ng Exendin-4+100 ng B-cellulin per well).

D13 and 16

5. Top up with RPMI/20SR (0.5 ml)+LF (100 ng HGF+20 ng Exendin-4+100 ng B-cellulin per well).

D20

6. Collect all MATRIGEL™ EB using 2-ml Pasteur pipette to a 15-ml falcon tube. Top up with pre-chilled PBS to 12 ml. Mix well and leave tubes on ice for 10 min.

7. Spin down MATRIGEL™ EB at 2500 rpm, 4 min. in the cool room.

8. Remove supernatant medium very carefully with 1 ml blue pipette. Do not disturb MATRIGEL™ EB.

9. Top up with pre-chilled PBS to 12 ml.

10. Spin down MATRIGEL™ EB at 2500 rpm, 4 min. in the cool room.

11. Transfer MATRIGEL™ EB to a 1.5-ml EPPENDORF tube using 2 ml Pasteur pipette.

For Immunohistochemistry Study—Paraffin Section

1) Spin gently to get EB pellets. Note the spin needs to be gentle and brief to keep good morphology of EBs.

2) Fix EBs with 4% PFA (paraformaldyhyde) for 2-4 hours at room temperature in 1.5-ml EPPENDORF tube

3) Wash with PBS 3 times-5 min. each.

4) Gently spin down EB pellets.

5) Melt 1.5% agarose at 60° C.

6) Resuspend EBs with 50-100 μl of melted agarose carefully and quickly.

7) Leave EPPENDORF tube on ice for a few minutes to solidify agarose.

8) Samples are ready for paraffin embedding.

For Immunohistochemistry Study—Cryosection

1) Spin gently to get EB pellets. Note the spin needs to be gentle and brief to keep good morphology of EBs.

2) Make mold.

3) Resuspend EBs carefully with 50-100 μl of freezing solution carefully. Do not produce bubble.

4) Add EB solution to mold carefully. Do not produce bubble.

5) Freeze samples in liquid nitrogen.

6) Stock samples at −70° C. or −20° C.

7) Samples are ready for cryosection. Note: the slides need to start from fixation step.

for RNA Extraction

Use high speed to get EB pellet. (Refer to Qiagen kit protocol or Trizol protocol.)

Note:

1. Some EBs might not be lysed very well by RTL lysis buffer even with vigorous pipetting. In this case, RLT lysate or Trizol lysate is better to be kept at −70° C. for at least a few hours before extraction. This freeze and thaw cycle appears to help lysing.

2. Trizol method gives more than double amount higher of the final RNA yield. However, DNA shredding step needs to be thorough and DNAase treatment step is preferred to be 1 hour in order to get rid of the genomic DNA cleanly.

Culture conditions beyond the initial 20 day protocol, in which pdx-1-expressing cells were directed toward more mature insulin producing cell populations, will be referred to as the maturation procedure(s). Day 20 EBs were removed from their RPMI-MATRIGEL™ cultures by centrifugation and cold PBS washes, and transferred to a basal medium (DMEM/F12, 17 mM glucose (Glc), 2 mM glutamax, 8 mM HEPES, 2% B27 supplement, and 1× pen/strep) thought to promote the recovery and survival of beta cells. On day 26, the cells were cultured for a further 6 days in the same basal media supplemented with 20 ng/ml FGF-18 and 2 μg/ml heparin. Between days 32 and 36, the cells were cultured in basal media supplemented with FGF-18 (20 ng/ml), heparin (2 μg/ml), EGF (10 ng/ml), TGFα (4 ng/ml), IGF-I (30 ng/ml), IGF-II (30 ng/ml) and VEGF (10 ng/ml). On day 36, the EBs were plated onto fibronectin-coated (10 μg/ml) tissue culture plates in a new media mix (RPMI+glucose (11 mM), FBS (5%), glutamax (2 mM), HEPES (8 mM), 1× pen/strep, forskolin (10 μM), HGF (40 ng/ml), and PYY (200 ng/ml)). The final stage (days 4044) consisted of culture in a CMRL-based media (supplemented with glucose (5 mM), glutamax (2 mM), pen/strep (1×), exendin-4 (100 ng/ml) and nicotinamide (5 mM). A more detailed ESI 3D MATRIGEL™ Protocol+Differentiation Protocol is as follows:

Cells: hES cells (preferentially hES3) are digested by Collagenase (2×p100 confluent dishes to one 6 well dish).

Plating: Cells are first distributed to a 6-well low-attachment plate in MATRIGEL™ to start the 3D culture, according to QC “MATRIGEL™ EB Protocol” in RPMI-SR. After 20 days, cells are re-plated on low-attachment dishes; after another 16 days cells are plated on Fibronectin-coated standard 6-well plates.

Sampling: (A) collect RNA from one well at day 20 of the 3D MATRIGEL™ protocol; (B) collect supernatant before and 24 h after each medium change from start of the multi-step maturation protocol (10 μl are required for one ELISA well, take 100 μl medium and keep at −20), (C) collect RNA from wells at day 45.

Media Used & Growth Factor Treatments:

1. 10 days 3D MATRIGEL™ Protocol w/ EFs=D0-D10 (initiation protocol—EF phase)

2 ml/well ⅙ MATRIGEL™ in RPMI-SR medium: RPMI, 20% SR, 1× Penicillin/Streptomycin plus 1×50 ng/ml each Activin A, BMP2, BMP4, Nodal Medium feeding at D3 and D6 (add 500 μl RPMI/20SR with 2×GF concentrations)

Day 10: Medium Change; remove medium with 1 ml pipette under the dissecting microscope very slow and carefully. Do not suck away MATRIGEL™ cells. Give 3 ml of RPMI-SR, equilibrate 1 hr in the incubator, then remove again and give new medium with late GFs.

2. 10 days 3D MATRIGEL™ Protocol with LFs=D10-D20 (—LF phase)

2 ml/well ⅙ MATRIGEL™ in RPMI-SR medium: RPMI, 20% SR, Penicillin/Streptomycin 1×50 ng/ml Betacellulin, 50 ng/ml HGF, 10 ng/ml Exendin-4, 10 mM Nicotinamide Medium feeding at D13 and D16 (add 500 μl with 2×GF concentrations).

D21—replating on low attachment plates: spin cells at 600 g for 4 min. (swing bucket); carefully remove MATRIOEL™ with a pipette; Resuspend in new medium. Collect one RNA sample (=+control for 3D).

3. 6 days step 1 Multi-step Maturation Protocol—D20-D26

2 ml/well basal medium only, no GFs: DMEM/F12, 17 mM Glc, 2 mM Glutamax, 8 mM HEPES, 2% B27, Penicillin/Streptomycin 1× Medium change at D23 (spin cells at 600 g for 4 min. in swing bucket, give new medium).

4. 6 days step 2 Multi-step Maturation Protocol—D26-D32

2 ml/well basal medium+20 ng/ml FGF-18, 2 μg/ml Heparin Medium change at D29 (spin cells at 600 g for 4 min. in swing bucket, give new medium).

5. 4 days step 3 Multi-step Maturation Protocol—D32-D36

2 ml/well basal medium+20 ng/ml FGF-18, 2 μg/ml Heparin, 10 ng/ml EGF, 4 ng/ml TGF-α, 30 ng/ml IGF-I, 30 ng/ml IGF-II, 10 ng/ml VEGF Medium change at D34 (spin cells at 600 g for 4 min in swing bucket, give new medium).

D36—replating on Fibronectin: coat 6-well plates for 1 hr with 10 μg/ml Fibronectin in PBS, wash 2× with RPMI-SR, plate cells in new medium on coated plates.

6. 6 days step 4 Multi-step Maturation Protocol—D36-D40

2 ml/well new RPMI medium: RPMI+11 mM Glc, 5% FBS, 2 mM Glutamax, 8 mM HEPES, Penicillin/Streptomycin (1×), 10 μM Forskolin, 40 ng/ml HGF, 200 ng/ml PYY.

Medium change at D38 (spin the portion of cells that is not attached to Fibronectin at 600 g for 4 min. in swing bucket, while doing that give 1 ml of new medium on the attached cells to avoid drying out, give recovered cells in new medium).

7. 4 days Step 5 Multi-Step Maturation Protocol—D40-D44

2 ml/well new CMRL medium+5 mM Glc, 5% FBS, 2 mM Glutamax, Penicillin/Streptomycin 100 ng/ml Exendin-4, 5 mM Nicotinamide. Medium change at D42 (as explained for 6).

Tissue processing and embedding: Embryoid bodies were isolated from the 3D MATRIGEL™ by transferring the entire culture to a 15 ml falcon tube and chilling on ice. The EBs were pelleted by centrifugation in a swinging-bucket rotor (1500 RPM). The cell pellet was then rinsed twice in ice-cold PBS and centrifuged in a similar fashion to remove residual MATRIGEL™. EBs were fixed in 4% paraformaldehyde for 4 hours, rinsed twice in PBS, and then stored at 4° C. in 70% ethanol. EBs were then embedded in paraffin using standard dehydration/clearing/paraffin-embedding protocols with a Leica TP1020 automated tissue processor (2×1 hr each in 70%, 95%, 100% ethanol followed by 2×1 hr in xylenes and 4×1 hr in paraffin). Sections were cut at 5 μm, and stored long-term at 4° C. for eventual immunohistochemistry or in situ hybridizations. Pancreas tissue was similarly prepared but with extended fixation.

Immunohistochemistry: Immunostaining was performed according to standard protocols using the vectorshield colorometric (DAB) detection kit. Slides were first de-waxed 2×10 min. in xylene, then hydrated in a standard ethanol series (100%, 95%, 90%, 70% ethanol, then 2 times in PBS). For antigen retrieval, slides were slowly heated in 10 mM sodium citrate solution to 95° C. (approximately 9 minutes on the defrost setting of a conventional microwave). The slides were then slowly cooled to room temperature (about 30 minutes) and rinsed twice in PBS. Endogenous peroxidase activity was quenched with a 20 min incubation in 3% H₂O₂ followed by 2 rinses in PBS. The slides were blocked for 1 hr in PBS containing 1% BSA and 5% serum (corresponding to the species from which the secondary antibody was derived). The primary antibodies were diluted to the following concentrations: rabbit anti-Pdx-1 (1:30,000), guinea pig anti Pdx-1. (1:2000), and goat anti-Pdx-1 (1:40,000). For C-peptide immunostaining, rabbit anti-human C-peptide (Linco Research, lot#81(1P)) antibodies were used at a 1:5000 dilution. Slides were incubated with the diluted primary antibody overnight. The next day, the slides were rinsed twice in PBS, and a biotinylated goat-anti-rabbit secondary antibody was applied for 1 hr at room temperature. After 3 rinses in PBS, the ABC mixture (Vectashields) was placed on the sections (prepared by mixing 20 μl/ml reagent A and 20 μl/ml reagent B in PBS) for 30 min. then rinsed away with 3×PBS washes followed by addition of the DAB substrate (Vector Laboratories, SK-4100). The color reaction was monitored closely by microscopy, and was stopped by dipping the slide in water and then rinsing once in 1×PBS. The sections were then dehydrated and cleared in xylenes (Sigma) using standard protocols before mounting in a xylene-based permanent mounting media (DPX neutral mounting media, Sigma).

RNA isolation, cDNA synthesis, and quantitative PCR: Total RNA from hESC or EBs at various stages of differentiation was isolated using the Qiagen RNeasy kit or prepared using Trizol reagent (Invitrogen) according to the manufacturer's instructions. RNA was and quantified by UV absorption. 1 to 5 μg of RNA was DNase I treated and converted to cDNA using M-MuLV reverse transcriptase (New England Biolabs) using oligo-dT or random hexamer primers according to the manufacturer's instructions. Quantitative PCR was performed according to the manufacturer's instructions using a BioRad iCycler with approximately 50 ng cDNA per reaction containing 250 nM of each primer and 1×SYBR green master mix (Bio-Rad) and analyzed by Bio-RAD thermocycler.

The following conditions were used:

Quantitative PCR Reaction:

| | | |
|---|---|---|
| 2× master mix | 15 | μl |
| primers (each) | 100 | nM |
| template | 25 | ng |
| H₂O | to 30 | μl |

40 Cycles of:

30 s at 95° C.—denaturation

30 s at 55° C.—annealing

60 s at 72° C.—extension

Plate Setup:

For each unknown sample, include:

3 replicates from the same RT reaction.

1 sample that was treated identically except that the RT enzyme was not included in the RT reaction (no RT control). This is to control for genomic or other contamination (such as the previous PCR reaction still in your pipette nozzle). This should produce no signal before Ct=35.

For the genes on the following list, include 2 replicates each of 2 positive controls. Use 10 ul of each positive control per reaction. These are prepared such that the amount on the label is contained in 10 ul of solution.

| | Range | | | | | |
|---|---|---|---|---|---|---|
| Gene | quantity | Ct | quantity | | Ct | Slope* |
| Oct4 | 10 pg | 15 | 1 | fg | 29 | −1.7307 |
| Nanog | 10 pg | 13 | 1 | fg | 28 | −1.5994 |
| Sox17 | 10 pg | 15 | 100 | ag | 32 | −1.5147 |
| glut-2 | 10 pg | 16 | 100 | ag | 34 | −1.4216 |
| HNF3b | 10 pg | 13.5 | 100 | ag | 31 | −1.5167 |
| α Fetal Protein | 10 pg | 14.5 | 100 | ag | 34 | −1.7245 |
| Albumin | 10 pg | 14 | 1 | fg | 30 | −1.6 |
| Thans-thyretin | 10 pg | 20 | 100 | ag | 37 | −1.7245 |
| Cyt P450-3A4 | 10 pg | 15 | 100 | ag | 33 | −1.5576 |
| Trp dioxygenase | 10 pg | 13 | 1 | fg | 25 | −1.1193 |
| CK19 | 10 pg | 21 | 1 | fg | 31 | −1.2 |
| GGT | 10 pg | 20 | 10 | fg | 34 | −1.8669 |
| Pdx-1 | 10 pg | 15 | 100 | ag | 30 | −1.2703 |
| Insulin | 10 pg | 10 | 1 | fg | 25 | −1.5603 |
| Brachyury | 10 pg | 15 | 100 | fg | 26 | −2.4465 |
| tbx6 | 10 pg | 15 | 10 | fg | 30 | −2.1469 |
| sox-1 | 10 pg | 21 | 10 | fg | 34 | −1.8892 |
| Neurofilament, HC | 10 pg | 15 | 1 | fg | 29 | −1.4708 |
| β-actin | 10 pg | 15 | 1 | fg | 34 | −1.3897 |
| GAPDH | 10 pg | 12 | 1 | fg | 28 | −1.3978 |

Analysis of Gene Expression.

1) Cut and paste (or export) data to an excel spreadsheet.

2) Graph Ct (Y) vs. quantity (X) of standard curves. Convert X axis to log scale, Log(X). Get equation Y=slope*Log(X)+Y intercept. Under “options” include equation and R² value on chart.

3) Determine input of unknown sample using the following equation (this can be prepared in the excel spreadsheet):

4) Input=10ˆ((Ct value−y intercept)/slope).

5) Repeat this procedure for the internal control of gene expression (GAPDH or β-actin)

6) Calculate the average input value of the three replicates for the gene and the internal control gene.

7) Calculate normalized expression of your gene using the following equation: normalized expression=Input value average of gene/Input value average of internal control gene.

8) Calculate the relative expression of your gene: Set one experimental condition as the comparison sample (untreated or time=0, for example). Relative expression=Normalized expression of unknown/normalized expression of comparison.

Quality controls—

(a) The slope of the curve you generate from the positive controls should be roughly equal to the slope in the standard curve chart below. If not, prepare fresh primer mix and standard curve reagents. To compare slopes, the trendlines must be generated in the same way. The slopes generated here use quantity in femtograms.

(b) The Ct value of the unknown sample should be between the Ct values given by the positive controls. Otherwise, the results are outside the sensitive range of the assay.

Standard curves were generated by plotting the log (concentration in fg) of series of 100-fold dilutions of the target PCR amplicon (a range of 10⁴ fg to 1 fg per reaction) versus the corresponding threshold Ct value. Normalized expression was determined by the following equation: Normalized expression=(input of target gene/input of actin control), where input is calculated as the inverse log of ((the threshold cycle (Ct value)—Y-intercept of standard curve)/slope of standard curve).

The following specific primer pairs were used:

| Gene | | |
|---|---|---|
| name | Forward primer | Reverse primer |
| APP | GTAGCGCTGCAAACAATGAA | TCCAACAGGCCTGAGAAATC |
| | (SEQ ID NO: 1) | (SEQ ID NO: 2) |
| Oct4 | GGCAACCTGGAGAATTTGTT | GCCGGTTACAGAACCACACT |
| | (SEQ ID NO: 3) | (SEQ ID NO: 4) |
| Nanog | TACCTCAGCCTCCAGCAGAT | TGCGTCACACCATTGCTATT |
| | (SEQ ID NO: 5) | (SEQ ID NO: 6) |
| HNF3-b | GGAGCGGTGAAGATGGAA | TACGTGTTCATGCCGTTCAT |
| | (SEQ ID NO: 7) | (SEQ ID NO: 8) |
| Sox17 | CAGAATCCAGACCTGCACAA | CTCTGCCTCCTCCACGAA |
| | (SEQ ID NO: 9) | (SEQ ID NO: 10) |
| Glut-2 | CATGTCAGTGGGACTTGTGC | CTGGCCCAATTTCAAAGAAG |
| | (SEQ ID NO: 11) | (SEQ ID NO: 12) |
| albumin | TCAGCTCTGGAAGTCGATGA | TTCACGAGCTCAACAAGTGC |
| | (SEQ ID NO: 13) | (SEQ ID NO: 14) |
| Pdx-1 | CCTTTCCCATGGATGAAGTC | GGAACTCCTTCTCCAGCTCTA |
| | (SEQ ID NO: 15) | (SEQ ID NO: 16) |
| Insulin | GGGGAACGAGGCTTCTTCTA | CACAATGCCACGCTTCTG |
| | (SEQ ID NO: 17) | (SEQ ID NO: 18) |
| Glucagon | CCAAGATTTTGTGCAGTGGT | GGTAAAGGTCCCTTCAGCAT |
| | (SEQ ID NO: 19) | (SEQ ID NO: 20) |
| somato- | CCCAGACTCCGTCAGTTTCT | ATCATTCTCCGTCTGGTTGG |
| statin | (SEQ ID NO: 21) | (SEQ ID NO: 22) |
| Pax4 | TCTCCTCCATCAACCGAGTC | GAGCCACTATGGGGAGTGAG |
| | (SEQ ID NO: 23) | (SEQ ID NO: 24) |
| Cyp450- | ACCGTGACCCAAAGTACTGG | GTTTCTGGGTCCACTTCCAA |
| 3A4 | (SEQ ID NO: 25) | (SEQ ID NO: 26) |
| brachy- | AATTGGTCCAGCCTTGGAAT | CGTTGCTCACAGACCACAG |
| rury | (SEQ ID NO: 27) | (SEQ ID NO: 28) |

C-peptide ELISA: C-peptide concentrations in conditioned medium were determined by ELISA with commercially available anti-C-peptide-coated plates (LINCO research) according to the manufacturer's recommendations.

Riboprobe synthesis: Template plasmids were linearized with either Hind III (antisense) or BamH I (sense), and then purified (Qiagen Qiaquick spin columns). 1.5 μg of recovered DNA template was used in a synthesis reaction containing the corresponding RNA polymerase (Promega), nucleotides (DIG RNA labeling mix—10 mM ATP, CTP, GTP (each), 6.5 mM UTP, 3.5 mM DIG-11-UTP), 1× Transcription buffer (1×), and 25 Units RNAsin (Promega). RNA probes were precipitated by the addition of 0.1 volumes of 4 M LiCl and 2.5 volumes of 100% ethanol and incubated at −20° C. overnight. Samples were then centrifuged at 13,000 rpm for 30 min. at 4° C. The supernatant was discarded and the pellet was washed with 70% ethanol:30% DEPC-H₂O and re-centrifuged for 15 min. The supernatant was removed, and the pellet allowed to dry. Probes were typically resuspended in 50 μl of DEPC-H₂O, aliquoted and stored at −80° C.

In Situ Hybridization: EBs were rehydrated in a descending series of methanol: PBT washes (75%, 50%, and 25% methanol). PBT is prepared from 1×PBS-DEPC plus 0.1% Tween-20. EBs were incubated for 1 hr in 6% H₂O₂ in PBT and then rinsed 3× in PBT. EBs were then treated for 5 min. in 10 μg/ml proteinase K in PBT, washed in 2 mg/ml glycine in PBT (5 min.), followed by 2 additional PBT washes (5 min. each), and re-fixed in 4% paraformaldehyde (Sigma)/0.2% glutaraldehyde/PBT for 20 min. at room temperature. EBs were incubated in hybridization solution (50% deionized formamide (Ambion), 5×SSC, 0.1% Tween-20 (Sigma), 0.1% SDS (Sigma), 50 μg/ml heparin (Sigma), 50 μg/ml yeast tRNA, 60 mM citric acid in DEPC treated H₂O) for at least two hours at 70° C. Hybridization solution was then replaced with fresh solution containing 50-100 ng DIG-labeled riboprobe and incubated on a rocking platform overnight at 70° C. The following day, EBs were washed for 5 min. in Solution 1 (50% formamide, 5×SSC, 60 mM citric acid, and 1% SDS in DEPC-treated H₂O) pre-warmed to 70° C. EBs were washed twice more in solution I for 30 min. each at 70° C., and once in solution I for 30 min. at 65° C. EBs were then washed 3× in Solution II (50% formamide, 2×SSC, 24 mM citric acid, 0.2% SDS, and 0.1% Tween-20 in DEPC-treated H₂O) for 30 min. each at 65° C. EBs were cooled to room temperature and washed 3×(5 min. each) in maleic acid buffer (100 mM maleic acid (Sigma), 170 mM NaCl (Sigma), 0.1% Tween-20, and 2 mM levamisole, pH 7.5 with NaOH) (MAB). EBs were then incubated for 90 min at room temperature in blocking solution (MAB, 2% Boehringer Mannheim blocking reagent, 10% heat inactivated sheep serum). Blocking solution was then replaced with fresh blocking solution containing pre-adsorbed alkaline phosphatase (AP)-conjugated anti-digoxygenin antibody (Roche) and incubated on a rocking platform overnight at 4° C. 2 μl antibody per ml was pre-adsorbed by incubation in MAB with 2% Boehringer Mannheim blocking reagent, 1% heat inactivated sheep serum, and 3 mg human EB acetone powder at 4° C. for 90 min. and centrifuged for 10 min. at 4° C. EBs were washed 3×(5 min. each) and 5×(60-90 min. each) in MAB at room temperature and then incubated overnight in MAB at 4° C. The following day, EBs were washed 3×(10 min. each) in AP buffer (100 mM Tris-HCl, 100 mM NaCl, 50 mM MgCl₂, 0.1% Tween-20, and 2 mM levamisole). EBs were then incubated in NTMT alkaline phosphatase staining buffer (AP buffer with 3.5 μl/ml NBT and 3.5 μl/ml BCIP) or alkaline phosphatase staining solution (BM Purple, Boehringer Mannheim) until the precipitation reaction was complete. Reaction was arrested with the addition of stop solution (2 mM EDTA in PBT).

### ADDITIONAL REFERENCES

Reubinoff et al. (2000) Nat Biotechnol 18: 399-404.
Miyazaki et al. (2004) Diabetes 53: 1030-1037
Schuldiner and Benvenisty. (2001) Recent Res. DeveL Mol. Cell. Biol 2: 223-231.
Itskovitz-Eldor et al. (2000) Mol Med 6: 88-95.
Reubinoff et al. (2001) Nat Biotechnol 19: 1134-40.
Carpenter et al. (2001) Exp Neurol 172: 383-97.
Goldstein et al. (2002) Developmental Dynamics 225: 80-86.
Schuldiner et al. (2001) Brain Research 913: 201-205.
Pan et al. (2002) Cell Research 12: 321-329.
Eiges and Benvenisty (2002) FEBS Letters 529: 135-141.
Schuldiner et al. (2000) PNAS 97: 11307-11312.
Shamblott et al. (2001) PNAS 98: 113-118.
Thomson et al. (1998) Science 282: 1145-1147.
Pesce and Scholer (2001) Stem Cells 19: 271-278.
Pesce and Scholer (2000) Mol Rep Dev 55: 452-457.
Nichols et al. (1998) Cell 95: 379-391.
Niwa et al. (2000) Nature Genetics 24: 372-376.
Hansis et al. (2000) Mol Hum Reprod 6: 999-1004.
Palumbo et al. (2002) Journal of Pathology 196: 467-477.
Monk. (2001) Oncogene 20: 8085-8091.
Jin et al. (1999) International Journal of Cancer 81: 104-112.
Kohlhase et al. (2002) Human Molecular Genetics 11: 2979-2987.
Al-Baradie et al. (2002) American Journal of Human Genetics 71: 1195-1199.
Zhang et al. (2001) Nat Biotechnol 19: 1129-33.
Kehat et al. (2001) J Clin Invest 108: 407-14.
Mummery et al. (2002) J Anat 200: 233-42.
Kaufman et al. (2001) Proc Natl Acad Sci USA 98: 10716-21.
Eiges et al. (2001) Curr Biol 11: 514-8.
Henderson et al. (2002) Stem Cells 20: 329-37.
Ramalho-Santos et al. (2002) Science 298: 597-600.
Ivanova et al. (2002) Science 298: 6014.
Eisen et al. (1998) Proc Natl Acad Sci USA 95: 14863-8.
Hamada et al. (2002) Nat Rev Genet 3: 103-13.
Leahy et al. (1999) J Exp Zool 284: 67-81.
Pelton et al. (2002) J Cell Sci 115: 329-39.
Gillespie and Uversky. (2000) Biochim Biophys Acta 1480: 41-56.
Crouch. (1998) Biochim Biophys Acta 1408: 278-89.
Ashizawa et al. (2004) Pancreas 28: 109-120.
Assady et al. (2001) Diabetes 50: 1691-1697.
Chen et al. (2004) Dev Biol 271, 144-160.
Dean and McEntyre (2004) Chapter 1: Introduction to diabetes. In The genetic landscape of diabetes, L. Dean, and J. R. McEntyre, eds. (Bethesda (Md.), National library of medicine (US)).
ESI (2003) Methodology manual human embryonic stem cell culture. Handbook, 1-25.
Hansson et al. (2004) Diabetes 53, 2603-2609.
Hebrok. (2003) Mech Dev 120, 45-57.
Hebrok et al. (2000) Development 127, 4905-4913.
Jonsson et al. (1994) Nature 371, 606-609.
Kahan et al. (2003) Diabetes 52, 2016-2024.
Kim and Melton (1998) Proc Natl Acad Sci USA 95, 13036-13041.
Kishi et al. (2003) Am J Physiol Endocrinol Metab 284, E830-E840.
Kumar and Melton (2003) Curr Opin Genet Dev 13, 401-407.
Kundu et al. (2003) Cell Struct Funct 28, 255-263.
Lumelsky et al. (2001) Science 292, 1389-1394.
Rajagopal et al (2003 Science 299, 363.
Ryan et al. (2002) Diabetes 51, 2148-2157.
Segev et al. (2004). Stem Cells 22, 265-274.
Slack. (1995) Development 121, 1569-1580.
Soria. (2001) Differentiation 68, 205-219.
U.S. Pat. No. 6,613,568
U.S. Pat. No. 6,602,711
US 2003/0068819
US 2003/0190748
US 2004/0023376
US 2004/0043484
US 2004/0106095
WO04/072251
WO00/70021
WO02/10347
WO02/061033
US 2003/0224345
US 2004/0014210

The practice of the present invention will employ, unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

All publications, patents and patent applications are herein incorporated by reference in their entirety to the same extent as if each individual publication, patent or patent application was specifically and individually indicated to be incorporated by reference in its entirety.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for directed differentiation of embryonic stem (ES) cells into pancreatic lineage, comprising: contacting the ES cells for a sufficient period of time with a sufficient amount of one or more early factors (EFs) selected from activin A, BMP2, BMP4, or nodal, wherein the pancreatic lineage cells express pancreatic lineage marker(s), and/or exhibit a pancreatic lineage function.

2. The method of claim 1, wherein the pancreatic lineage cells express Pdx-1 and/or insulin, and/or are responsive to glucose, and/or secret C-peptide.

3. The method of claim 2, wherein the pancreatic lineage cells are Insulin-producing cells.

4. The method of claim 1, wherein the ES cells are cultured as embryoid bodies (EBs), plated directly onto a support matrix, and/or plated directly onto tissue culture plates.

5. The method of claim 4, wherein the EBs are cultured in a floating suspension culture, in a support matrix, and/or on a filter.

6. The method of claim 4, wherein the EBs are cultured in a support matrix only during the period when the EBs are in contact with the EFs.

7. The method of claim 4, wherein the support matrix is MATRIGEL™.

8. The method of claim 4, wherein the EBs are generated from ES cells grown on MEF (mouse embryonic feeder) or other feeder layers, or from ES cells grown under feeder-free conditions.

9. The method of claim 1, wherein the ES cells are human ES cells.

10. The method of claim 1, wherein the ES cells are mouse ES cells.

11. The method of claim 1, wherein the ES cells are partially or terminally differentiated into the pancreatic lineage.

12. The method of claim 1, wherein the ES cells are contacted with the EFs for about 15 days.

13. The method of claim 1, wherein the EFs comprise activin A and BMP4.

14. The method of claim 1, wherein the EFs comprise about 50 ng/mL of activin A and about 50 ng/mL of BMP4.

15. The method of claim 1, further comprising contacting the ES cells, subsequent to contacting the ES cells with the EFs, with a sufficient amount of one or more late factors (LFs) for a second sufficient period of time.

16. The method of claim 15, wherein the one or more LFs are HGF, exendin4, betacellulin, and nicotinamide.

17. The method of claim 15, wherein the one or more LFs include about 50 ng/mL of HGF, about 10 ng/mL of exendin4, and about 50 ng/mL of β-cellulin.

18. The method of claim 15, wherein the ES cells are contacted with the EFs for about 10 days, and are subsequently contacted with the LFs for about 10 days.

19. The method of claim 18, wherein the EFs comprise about 50 ng/mL of activin A and about 50 ng/mL of BMP4, and the LFs include about 50 ng/mL of HGF, about 10 ng/mL of exendin4, and about 50 ng/mL of β-cellulin.

20. The method of claim 15, further comprising contacting the ES cells, subsequent to the initiation protocol and during a maturation protocol, consecutively with:
(1) a basal medium for about 6 days;
(2) about 20 ng/ml FGF-18, and about 2 μg/ml Heparin in the basal medium for about 5-6 days;
(3) about 20 ng/ml FGF-18, about 2 μg/ml Heparin, about 10 ng/ml EGF, about 4 ng/ml TGFα, about 30 ng/ml IGF1, about 30 ng/ml IGF2, and about 10 ng/ml VEGF in the basal medium for about 4-5 days;
(4) about 10 μM Forskolin, about 40 ng/ml HGF, and about 200 ng/ml PYY for about 3-4 days; and,
(5) about 100 ng/ml Exendin-4, and about 5 mM Nicotinamide for about 3-4 days.

21. The method of claim 20, wherein the ES cells are not dissociated by dispase between step (1) and (2).

22. The method of claim 20, wherein FBS (if any) in the medium is replaced with a chemically defined serum replacer (SR).

23. The method of claim 15, further comprising contacting the ES cells, subsequent to the EF and LF treatment, and during a maturation protocol, with about 10 μM Forskolin, about 40 ng/ml HGF, and about 200 ng/ml PYY for about 3-4 days.

24. The method of claim 23, wherein the ES cells are grown on fibronectin-coated tissue culture surfaces during the maturation protocol.

25. The method of claim 23, wherein the differentiated cells release C-peptide and/or are responsive to glucose stimulation.

26. The method of claim 23, wherein FBS (if any) in the medium is replaced with a chemically defined serum replacer (SR).

27. The method of claim 1, further comprising contacting the ES cells, subsequent to contacting the ES cells with the EFs and during a maturation protocol, consecutively with:
(1) about 20 ng/ml FGF-18, and about 2 μg/ml Heparin in a basal medium for about 8 days;
(2) about 20 ng/ml FGF-18, about 2 μg/ml Heparin, about 10 ng/ml EGF, about 4 ng/ml TGFα, about 30 ng/ml IGF1, about 30 ng/ml IGF2, and about 10 ng/ml VEGF in the basal medium for about 6 days; and
(3) about 10 μM Forskolin, about 40 ng/ml HGF, and about 200 ng/ml PYY for about 5 days.

28. The method of claim 27, wherein the differentiated cells release C-peptide.

29. The method of claim 27, wherein step (1) lasts 6 days, steps (2) and (3) last 4 days each.

30. Differentiated pancreatic lineage cells or cell cultures obtained through the method of claim 1.

31. Differentiated pancreatic lineage cells or cell cultures obtained through the method of claim 2.

32. The differentiated pancreatic lineage cells or cell cultures of claim 31, which are partially differentiated.

33. The differentiated pancreatic lineage cells or cell cultures of claim 31, which are terminally differentiated.

34. The differentiated pancreatic lineage cells or cell cultures of claim 31, which mimic the function, in whole or in part, of Insulin-producing cells.

35. Differentiated pancreatic lineage cells or cell cultures obtained through the method of claim 15.

36. Differentiated pancreatic lineage cells or cell cultures obtained through the method of claim 20.

37. Differentiated pancreatic lineage cells or cell cultures obtained through the method of claim 23.

38. Differentiated pancreatic lineage cells or cell cultures obtained through the method of claim 27.

39. A method for the treatment or prophylaxis, in an individual, of diseases, injuries, or conditions of the pancreas characterized by impaired pancreatic function, comprising administering to the individual the differentiated pancreatic lineage cells of claim 30.

40. The method of claim 39, wherein the impaired pancreatic function includes impaired ability to properly regulate glucose metabolism in an affected individual.

41. The method of claim 39, wherein the condition is type I or type II diabetes.

42. The method of claim 39, which is in conjunction with one or more additional therapies effective for the treatment or prophylaxis of the diseases, injuries, or conditions.


SEQUENCE LISTING (28 sequences)

SEQ ID NO: 1 - DNA, 20 bp, Artificial Sequence
  PCR forward primer for AFP
gtagcgctgcaaacaatgaa

SEQ ID NO: 2 - DNA, 20 bp, Artificial Sequence
  PCR reverse primer for AFP
tccaacaggcctgagaaatc

SEQ ID NO: 3 - DNA, 20 bp, Artificial Sequence
  PCR forward primer for Oct 4
ggcaacctggagaatttgtt

SEQ ID NO: 4 - DNA, 20 bp, Artificial Sequence
  PCR reverse primer for Oct 4
gccggttacagaaccacact

SEQ ID NO: 5 - DNA, 20 bp, Artificial Sequence
  PCR forward primer for Nanog
tacctcagcctccagcagat

SEQ ID NO: 6 - DNA, 20 bp, Artificial Sequence
  PCR reverse primer for Nanog
tgcgtcacaccattgctatt

SEQ ID NO: 7 - DNA, 18 bp, Artificial Sequence
  PCR forward primer for HNF3-b
ggagcggtgaagatggaa

SEQ ID NO: 8 - DNA, 20 bp, Artificial Sequence
  PCR reverse primer for HNF3-b
tacgtgttcatgccgttcat

SEQ ID NO: 9 - DNA, 20 bp, Artificial Sequence
  PCR forward primer for Sox 17
cagaatccagacctgcacaa

SEQ ID NO: 10 - DNA, 18 bp, Artificial Sequence
  PCR reverse primer for Sox 17
ctctgcctcctccacgaa

SEQ ID NO: 11 - DNA, 20 bp, Artificial Sequence
  PCR forward primer for Glut-2
catgtcagtgggacttgtgc

SEQ ID NO: 12 - DNA, 20 bp, Artificial Sequence
  PCR reverse primer for Glut-2
ctggcccaatttcaaagaag

SEQ ID NO: 13 - DNA, 20 bp, Artificial Sequence
  PCR forward primer for albumin
tcagctctggaagtcgatga

SEQ ID NO: 14 - DNA, 20 bp, Artificial Sequence
  PCR reverse primer for albumin
ttcacgagctcaacaagtgc

SEQ ID NO: 15 - DNA, 20 bp, Artificial Sequence
  PCR forward primer for Pdx-1
cctttcccatggatgaagtc

SEQ ID NO: 16 - DNA, 21 bp, Artificial Sequence
  PCR reverse primer for Pdx-1
ggaactccttctccagctcta

SEQ ID NO: 17 - DNA, 20 bp, Artificial Sequence
  PCR forward primer for Insulin
ggggaacgaggcttcttcta

SEQ ID NO: 18 - DNA, 18 bp, Artificial Sequence
  PCR reverse primer for Insulin
cacaatgccacgcttctg

SEQ ID NO: 19 - DNA, 20 bp, Artificial Sequence
  PCR forward primer for Glucagon
ccaagattttgtgcagtggt

SEQ ID NO: 20 - DNA, 20 bp, Artificial Sequence
  PCR reverse primer for Glucagon
ggtaaaggtcccttcagcat

SEQ ID NO: 21 - DNA, 20 bp, Artificial Sequence
  PCR forward primer for somatostatin
cccagactccgtcagtttct

SEQ ID NO: 22 - DNA, 20 bp, Artificial Sequence
  PCR reverse primer for somatostatin
atcattctccgtctggttgg

SEQ ID NO: 23 - DNA, 20 bp, Artificial Sequence
  PCR forward primer for Pax 4
tctcctccatcaaccgagtc

SEQ ID NO: 24 - DNA, 20 bp, Artificial Sequence
  PCR reverse primer for Pax 4
gagccactatggggagtgag

SEQ ID NO: 25 - DNA, 20 bp, Artificial Sequence
  PCR forward primer for Cyp450-3A4
accgtgacccaaagtactgg

SEQ ID NO: 26 - DNA, 20 bp, Artificial Sequence
  PCR reverse primer for Cyp450-3A4
gtttctgggtccacttccaa

SEQ ID NO: 27 - DNA, 20 bp, Artificial Sequence
  PCR forward primer for brachyrury
aattggtccagccttggaat

SEQ ID NO: 28 - DNA, 19 bp, Artificial Sequence
  PCR reverse primer for brachyrury
cgttgctcacagaccacag